(19) United States
(12) Patent Application Publication
(10) Pub. No.: US 2023/0382951 A1
(43) Pub. Date: Nov. 30, 2023

(54) BRAIN PENETRATING PEPTIDES

(71) Applicant: BOARD OF REGENTS, THE UNIVERSITY OF TEXAS SYSTEM, Austin, TX (US)

(72) Inventors: Debadyuti GHOSH, Austin, TX (US); Xiujuan PENG, Austin, TX (US)

(73) Assignee: BOARD OF REGENTS, THE UNIVERSITY OF TEXAS SYSTEM, Austin, TX (US)

(21) Appl. No.: 18/250,107

(22) PCT Filed: Oct. 21, 2021
(86) PCT No.: PCT/US21/55948

§ 371 Date: Apr. 21, 2023

Related U.S. Application Data
(60) Provisional application No. 63/104,210, filed on Oct. 22, 2020.

(51) Int. Cl. C07K 7/64; C07K 7/08; A61K 47/68; A61K 47/64

(52) U.S. Cl. CPC C07K 7/64; C07K 7/08; A61K 47/6849; A61K 47/64; A61K 38/00

(57) ABSTRACT

The present disclosure is directed to the identification of peptides that cross the blood brain barrier and their use to transport diagnostic and therapeutic payloads into the brain.

### PRIORITY CLAIM

This application claims benefit of priority to U.S. Provisional Application Ser. No. 63/104,201, filed Oct. 22, 2020, the entire contents of which are hereby incorporated by reference.

### BACKGROUND

### 1. Field of the Disclosure

The present disclosure relates generally to the fields of medicine, cell biology and neurology. More particular, the disclosure relates to the identification of peptides that facilitate the transfer of attached materials across the blood-brain barrier.

### 2. Background

Over 1 billion people globally suffer from neurological diseases, which account for 12% of total deaths annually¹. Unfortunately, therapeutic delivery of drugs to the central nervous system (CNS) and into the brain parenchyma has remained a long-standing and significant challenge for CNS diseases²⁻⁴. Upon their administration, drugs must penetrate multiple barriers such as the blood-brain barrier (BBB), the perivascular space, the cerebrospinal fluid, and the extracellular space surrounding the cells of the brain to reach target sites⁵. In systemic delivery, the BBB is the primary barrier to the brain.

The complex molecular composition and the size capacity of the BBB and the ECS limit drugs and nanoscale delivery systems that possess the desired physicochemical properties to traverse these barriers. For example, nanocarriers greater than 150 nm in diameter are unable to penetrate the intact BBB²⁵ and diffuse through the ECS^{19,26} due to their size. Also, while positively charged solutes can bind and enter the brain capillary endothelium, they are unable to efficiently dissociate from the plasma membrane and diffuse unhindered across the negatively charged ECS^{18,19,21}. Strategies such as intracerebral injection, hyperosmotic disruption, convection enhanced drug delivery, ultrasound-induced microbubble-mediated delivery, intranasal delivery, and functionalized nanoparticles²⁷⁻³² have been used to transiently open, shuttle or bypass the BBB, but they have not yet achieved drug delivery in the CNS at therapeutic concentrations and/or require local delivery or a permeable BBB, which raises potential concerns over their safety.

One promising strategy to identify BBB-penetrating drug carriers is phage display technology. Phage, which are bacterial viruses, can be genetically engineered to display peptides or proteins on their surface. In particular, M13 phage are filamentous nanoparticles (˜900 nm in length and 6-7 nm in diameter) that present a collection, or library, of random peptides. Phage libraries have been previously used to identify BBB shuttle peptides in vitro and in vivo, but there are challenges in their use. Traditional panning in vitro using phage display identified peptides that bind but may not transcytose the BBB and diffuse through the ECS. Also, most BBB-shuttle peptides were identified using Sanger sequencing, which covers a limited sample space (5-1000 clones)^{33,34}. For example, the SGV motif was identified from in vitro biopanning by Sanger sequencing of 31 clones³⁴; however, this motif was not validated in vivo, and the other identified sequences were not validated. CRT³⁸, TGN³⁹, and PepC7⁴⁰ peptides were identified from in vivo panning in different rodent models, but in vivo panning can also restrict the number acquired after multiple rounds of selection (12-500 clones)³³. These BBB shuttle peptides demonstrated slightly improved delivery of solid lipid nanoparticles (SLN) into the brain. Unfortunately, these peptides did not significantly improve the therapeutic efficacy of SLN. As a result, there is a limited sequence space to identify brain penetrating peptides. Several other peptides, including HAI³⁵, Peptide 22³⁶, were discovered from library biopanning against the human transferrin receptor (hTfR) and low-density lipoprotein receptor (hLDLR), which were able to transcytose across the BBB but not with high efficiency^{35,36}. TfR and LDLR are present in other tissues besides the brain, however, and there is the potential for non-specific uptake in these tissues, leading to off-target effects³⁷. The peptides discovered through in vivo panning are against targets that may have different expression levels in humans⁴¹. Also, in vivo panning may identify suboptimal peptides; since phages have a short half-life in systemic circulation^{42,43}, it is possible that candidate peptides do not have sufficient time to bind and traverse the BBB. As a result, the first round of in vivo selection becomes paramount to identify successful BBB penetrating peptides⁴⁴. From these potential challenges, there is a need to discover new BBB and ECS penetrating peptides from a larger sequence space using in vitro phage display with next-generation sequencing (NGS).

### SUMMARY

Thus, in accordance with the present disclosure, there is provided a peptide of from 7 to 25 amino acid residues comprising and comprising a sequence selected from SEQ ID NOS: 1-20, wherein said peptide further comprises or is linked to one or more of:
(a) a non-natural amino acid;
(b) a D-amino acid;
(c) a non-amino acid chemical feature; and/or
(d) a therapeutic or diagnostic payload.

The peptide may comprise one or more non-natural amino acid. The peptide may comprise a D-amino acid, or more than one D-amino amino acid, including wherein the peptide comprises only D-amino-acids. The non-amino acid chemical feature may be polyethylene glycol or a linking agent. The payload may be a therapeutic payload or a diagnostic payload. The peptide may comprise (a) and (b); (a) and (c); (a) and (d); (b) and (c); (b) and (d); (c) and (d); (a), (b) and (c); (a), (c) and (d); (a), (b) and (d); (b), (c) and (d); or (a), (b), (c) and (d).

The peptide may be 8-25 residues in length, 9-25 residues in length, 10-25 residues in length, 12-25 residues in length, 15-25 residues in length or 20-25 residues in length. The peptide may be 8-20 residues in length, 9-20 residues in length, 10-20 residues in length, 12-20 residues in length, or 15-20 residues in length. The peptide may be 8-15 residues in length, 9-15 residues in length, 10-15 residues in length, or 12-15 residues in length. The peptide may be 8, 9, 10, 11, 12, 13, 14, 15, 16, 17, 18, 19, 20, 21, 22, 23, 24 or 25 residues in length. The peptide may be 7 residues in length, may comprise at least one D-amino acid, and optionally may be 9 residues in length, cyclized, such as through N- and C-terminal cysteine residues.

In another embodiment, there is provided a method of delivering a therapeutic or diagnostic payload across the blood-brain barrier of a subject comprising administering to said subject a peptide of from 7 to 25 amino acid residues comprising and comprising a sequence selected from SEQ ID NOS: 1-20, wherein said peptide is linked to a therapeutic or diagnostic payload. The method peptide may comprise one or more of:
(a) a non-natural amino acid;
(b) a D-amino acid; and/or
(c) a non-amino acid chemical feature.

The peptide may comprise one or more non-natural amino acid. The peptide may comprise a D-amino acid. The peptide may have more than one D-amino amino acid, such as comprising only D-amino-acids. The non-amino acid chemical feature may be polyethylene glycol or a linking agent. The payload may be a therapeutic payload or a diagnostic payload. The peptide may comprise (a) and (b); (a) and (c); (b) and (c); or (a), (b) and (c).

The peptide may be 8-25 residues in length, 9-25 residues in length, 10-25 residues in length, 12-25 residues in length, 15-25 residues in length or 20-25 residues in length. The peptide may be 8-20 residues in length, 9-20 residues in length, 10-20 residues in length, 12-20 residues in length, or 15-20 residues in length. The peptide may be 8-15 residues in length, 9-15 residues in length, 10-15 residues in length, or 12-15 residues in length. The peptide may be 8, 9, 10, 11, 12, 13, 14, 15, 16, 17, 18, 19, 20, 21, 22, 23, 24 or 25 residues in length. The peptide may be 7 residues in length, comprises at least one D-amino acid, carries a therapeutic or diagnostic payload, and optionally is 9 residues in length, cyclized, such as through N- and C-terminal cysteine residues.

In yet another embodiment, there is provide a method of treating a disease or disorder in a subject comprising administering to said subject a peptide of from 8 to 25 amino acid residues comprising and comprising a sequence selected from SEQ ID NOS: 1-20, wherein said peptide is linked to a therapeutic payload. The disease or disorder may be a neurologic disease such as Alzheimer's Disease or Parkinson's Disease, may be stroke or traumatic brain injury, or may be cancer, such as a glioma, a craniopharyngioma, a lymphoma, a haemangioblastoma, a meningioma, an acoustic neuroma, a pineal region tumor, a pituitary tumor, or a primitive neuroectodermal tumor. The peptide may be administered orally, intravenously, intra-arterially, subcutaneously, or intramuscularly. The peptide may be administered to said subject more than once, such as daily, every other day, every three days, twice-weekly, weekly, every other week, or monthly, or on a chronic basis.

The peptide may further comprise one or more of:
(a) a non-natural amino acid;
(b) a D-amino acid; and/or
(c) a non-amino acid chemical feature.
   The peptide may be 7 residues in length, comprises at least one D-amino acid, carries a therapeutic or diagnostic payload, and optionally is 9 residues in length, cyclized, such as through N- and C-terminal cysteine residues.

In a further embodiment, there is provided a method of diagnosing a disease or disorder in a subject comprising administering to said subject a peptide of from 8 to 25 amino acid residues comprising and comprising a sequence selected from SEQ ID NOS: 1-20, wherein said peptide is linked to a diagnostic payload. The disease or disorder may be a neurologic disease such as Alzheimer's Disease or Parkinson's Disease, stroke or traumatic brain injury, or cancer. The peptide may be administered orally, intravenously, intra-arterially, subcutaneously, or intramuscularly. The peptide may further comprise one or more of:
(a) a non-natural amino acid;
(b) a D-amino acid; and/or
(c) a non-amino acid chemical feature.
   The peptide may be 7 residues in length, may comprise at least one D-amino acid, may carry a therapeutic or diagnostic payload, and optionally may be 9 residues in length, cyclized, such as through N- and C-terminal cysteine residues.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Scheme of CX7C peptide-presenting M13 phage library biopanning against in vitro BBB model and identification of peptide sequences. For the transcytosis assay, 10¹¹ plaque forming units (pfu) of CX7C peptide-presenting M13 phage library were added to the confluent hCMEC/D3 cells cultured on the transwell system in replicates. M13 phage clones in the eluate were grown in E. coli bacteria to amplify and make more copies for subsequent rounds of panning. Also, at each round, the DNA from the amplified eluate was isolated, purified, and prepared for next-generation sequencing (NGS) analysis (SEQ ID NO: 28).

FIG. 2. Enrichment analysis of the 20 most frequent peptides from the three rounds of biopanning against hCMEC/D3 cells. Peptides denoted from Pep-1 to Pep-20 refer to the 20 most abundant CX7C peptides present in the third round of biopanning. The sequence counts for each CX7C peptide from each round of screening were calculated as described in the Materials and Methods.

FIG. 3. Transcytosis assay of the 11 most frequent peptides-presenting phage and controls. hCMEC/D3 cells were cultured to form tight and continuous monolayer. The equivalent amount of each peptide-presenting M13 phage (Pep-1 to Pep-11, negative control NC, and positive controls PC-1 (SEQ ID NO: 23) and PC-2 (SEQ ID NO: 24)) were added to the BBB model in the transwell system. The ratio of output phage that went across hCMEC/D3 to initial input phage was calculated to compare the transcytosis efficiency of each M13 clone. The transcytosis efficiency for all the validated clones varied within the range of 1.92×10⁻⁵ to 3.5×10⁻³.

FIG. 4. Cellular uptake and intracellular diffusion of the M13 clones in hCMEC/D3 cells. hCMEC/D3 cells were cultured to reach confluency in 12-well plates. Equivalent amount of M13 clones (input) were added to cells for 1 h at 37° C. The amount the clones accumulated intracellularly were quantified (output). The ratio of output to input represents the efficiency of cellular uptake for each clone, which is shown on the left y-axis (black-filled bars). 2D particle tracking method was used to monitor and calculate the trajectories of each clone trafficking inside of hCMEC/D3 cells. Multiple 30 s movies were recorded (20 frames/s) to track the motion of Alexa Fluor® 488 conjugated M13 clones inside of hCMEC/D3 cells. Then, three steps of processing methods were performed on the movies: (i) Identifying contiguous regions of pixels; (ii) Gaussian fitting; and (iii) building trajectories from coordinates. About 193-1299 qualified trajectories were chosen to calculate the mean-squared displacement (MSD) for each clone. The diffusion coefficient (D) data are shown with the pink-filled bars, with the scale on the right y-axis. (PC-1=SEQ ID NO: 23)

FIG. 5. Transcytosis assay of selected peptide-presenting M13 phage compared to their respective scrambled controls in hCMEC/D3 cells. The efficiency of transcytosis (i.e., output to input) of Pep-1, Pep-2, Pep-3, Pep-4, Pep-5, Pep-8, Pep-9, and motif-presenting M13 clones was calculated from their transcytosis in hCMEC/D3 cells in the transwell system. Then, the transcytosis efficiency of each clone was normalized by the efficiency of their respective scrambled control M13 clone.

FIG. 6. Temperature-dependent transcytosis assay of peptide-presenting M13 phages in hCMEC/D3 cells. Transcytosis assays were performed at 37° C. and 4° C. for selected M13 clones against confluent hCMEC/D3 cells. The transcytosis efficiency was calculated as the ratio of output to input phage for each clone.

FIG. 7. Transcytosis assay of 5′carboxyfluorescein (FAM)-conjugated CX7C, R9, and Angiopep-2 peptides against hCMEC/D3 cells. Ten nmol of each FAM-labeled CX7C peptides and controls were added to confluent hCMEC/D3 cells in the donor compartment of the 24-well transwell plate and incubated up to 120 min at 37° C. For each peptide at each time point, the fluorescence intensity of the receiving compartment was measured by a plate reader and the ratio was calculated relative to the fluorescence of the initial amount added to the donor compartment.

FIGS. 8A-H. Temperature-dependent transcytosis of the FAM-labeled peptides against hCMEC/D3 cells. Transcytosis assay of 10 nmol of each fluorescently labeled peptide was performed against hCMEC/D3 cells in the 24-well transwell plate at 37° C. and 4° C. up to 120 min. At each timepoint, the ratio of fluorescent intensity was calculated as in prior studies to represent the transcytosis efficiency. At each panel, the transcytosis efficiency of each peptide at 37° C. (black line) and 4° C. (red line) was plotted versus time (min).

FIG. 9. Diffusion assay of FAM-CX7C peptides and controls through ECM coated transwells. Twenty nmol (200 μL) of each peptide (Pep-1, Pep-3, Pep-4, Pep-5, Pep-8, Pep-9, R9, and Angiopep-2) were added to 2 mm thick Matrigel that was coated on the insert membrane of 24-well transwell plate and incubated up to 6 h. The ratio of output to input of FAM fluorescence intensity indicated the Matrigel-diffusion outcome for each peptide throughout the assay.

FIGS. 10A-C. Diffusion of FAM-labeled CX7C, R9, and Angiopep-2 peptides in Matrigel embedded in a six-channel microfluidic chamber slide. Six nmol (60 μL of 100 μM FAM-conjugated peptide) was added to the inlet reservoir of a microfluidic channel, while 60 μL basal cell culture medium was loaded to the other reservoir to establish sink conditions to drive diffusion. Olympus IX83 fluorescence microscope was calibrated and set up to record the fluorescent images of the whole field of each channel (one peptide per channel) using time-lapse mode at 30 min intervals up to 12 h, for a total of 25 fluorescent images for each channel. Custom Matlab scripts were written to calculate the mean diffusivity of each peptide through ECM from analyzing the images obtained from time-lapse imaging; mean diffusivity was shown in the table (above). (FIG. 10A) Schematic of experimental design of the diffusion assay of FAM-peptides in Matrigel-embedded microfluidic chamber slide. (FIG. 10B) Representative fluorescent images of each peptide loaded into the reservoir at 0 h. (FIG. 10C) Representative fluorescent images of each peptide migrating through the ECM-embedded channel at 12 h.

FIGS. 11A-D. Distribution of phage clones into the brain parenchyma in vivo. Six to eight weeks old Balb/c mice were injected intravenously with either genetically engineered phage displaying Pep-3, Pep-9, and NC (FIGS. 11A-B) or phage conjugated with biotinylated Pep-3, Pep-9 and Angiopep-2 (FIGS. 11C-D) for 30 min for brain distribution. (FIG. 11A) The ratio of brain parenchyma to serum (p L/g) of Pep-3, Pep-9, and NC phage clones. (FIG. 11B) The ratios of brain capillary/serum (μVL/g) of Pep-3, Pep-9, and NC phage clones were shown in bar plot. (FIG. 11C) The ratio of brain parenchyma to serum (μL/g) of phage conjugated with biotinylated Pep-3, Pep-9, and Angiopep-2. (FIG. 11D) The ratios of brain capillary/serum(μL/g) of phage with biotinylated Pep-3, Pep-9, and Angiopep-2 were shown in the bar plot. Ratios were calculated accounting for the individual brain weight. Bar plots presented with mean and standard deviation (P<0.05).

FIGS. 12A-B. Biodistribution of fluorescently labeled Pep-3, Pep-9 and Angiopep-2 peptides in vivo. Six to eight weeks old female Balb/c mice were injected with either 100 μL of 500 μM FAM-labeled Pep-3, Pep-9, Angiopep-2 or saline by tail vein injection (n=3 in each group). After 30 min, the whole body was perfused and fixed before tissue harvesting (see method section). All tissues were imaged using IVIS Xenogen at excitation/emission wavelengths 500 nm/540 nm. (FIG. 12A) the fluorescent images of the brain, lung, heart were set on the same color scale (radiance efficiency in p/sec/cm²/sr/(VW/cm²)) for each group. (FIG. 12B) Total radiant efficiency of ROIs in each group was calculated and analyzed by multiple t-tests, P<0.05.

FIG. 13. Enrichment of 20 most frequent peptides among the three rounds of biopanning in the second replicate (repertoire).

FIG. 14 Frequency distribution of 20 most frequent peptides in the original, naïve CX7C phage library.

FIG. 15. Permeability of hCMEC/D3 with and without peptide-presenting phage. FITC-dextran was used as a tracer molecule to measure permeability of hCMEC/D3 cells incubated with and without peptide-presenting phage. Permeability coefficient was calculated in each group and presented as a bar plot.

FIG. 16. Active transport of Alexa Fluor 488 conjugated peptide-presenting phage clones in hCMEC/D3 cells after 1 h uptake. The active transport velocity (μm/s) was plotted for each peptide presenting M13 clone.

FIGS. 17A-C. Intracellular movements of M13 clones. (FIG. 17A) Representative trajectories from five peptide-presenting M13 phage in the hCMEC/D3 cells. A series of images of the fluorescently labeled M13 phage particles were projected on the bottom of the trajectory box. These five trajectories, a trajectory exhibits two active transport sub-trajectories lasting for is and 5 s respectively (by visual inspection); b-e represent passive diffusion. (FIG. 17B) Mean squared displacement (MSD) curves of trajectory segments that were classified as passive diffusion. The D value represents the mean and standard deviation of the derived diffusion coefficients. (FIG. 17C) MSD curves of trajectory segments that were classified as active transport. The V value represents the mean and standard deviation of the derived velocities.

FIGS. 18A-B. Biodistribution of FAM-labeled Pep-3, Pep-9 and Angiopep-2 peptides in kidney and liver after 30 min circulation. (FIG. 18A) The fluorescent images of the kidney and liver were set to the same scale (units are radiance efficiency in p/sec/cm²/sr/(W/cm²)) for each group. (FIG. 18B) Total radiant efficiency from drawn regions of interest (ROIs) in each group was calculated and analyzed by multiple t-tests, P<0.05.

FIG. 19. Two steps conjugation to develop IgG-Pep9 formulation by copper-free click chemistry.

FIGS. 20A-B. SDS-PAGE of free IgG and IgG-Pep9 conjugate formulations (non-reduced and reduced).

FIGS. 21A-D. Deconvolved mass spectrum of non-reduced and reduced free IgG and IgG-Pep9 formulations.

FIGS. 22A-D. Size measurement of the IgG and IgG-Pep9 formulation by dynamic light scattering (DLS).

FIG. 23. Linear standard curve of IgG and IgG-Pep9 formulation determined by ELISA.

FIGS. 24A-C. In vivo brain and blood distribution of IgG and IgG-Pep9 (24 h after dosing).

FIG. 25. Total ion chromatogram (TIC) of reduced IgG-Pep9 formulation from the complete LC-MS analysis.

FIG. 26. Mass spectra of reduced IgG-Pep9 formulation in the selected high intensity region of TIC.

### DESCRIPTION OF ILLUSTRATIVE EMBODIMENTS

To address the challenges of delivery across the dynamic BBB and the ECS, discussed above, the inventors report here the identification of peptides that achieve both transport across the BBB and improved diffusion through the extracellular matrix (ECM). Cysteine constrained heptapeptide-presenting M13 phage libraries were panned against an established in vitro model of the BBB to identify phage clones that transported across the BBB. Through next-generation DNA sequencing and bioinformatics, select peptide sequences were identified and subsequently validated.

Select peptide-presenting phage can shuttle across the BBB in vitro and in vivo. Importantly, selected peptides demonstrate improved diffusive transport than gold standard nona-arginine⁴⁵ and clinically trialed Angiopep-2⁴⁶-4⁴⁸ peptides and have equivalent efficiency of transcytosis as Angiopep-2 in vitro. From in vivo studies, select peptide-presenting phage shuttled across the BBB and distributed to the brain parenchyma. Pep-9 conjugated to M13 phage exhibited greater accumulation in the brain parenchyma compared to Angiopep-2. Out of the structural context of phage, the free peptides Pep-3 and Pep-9 achieved higher distribution in the brain than Angiopep-2 and may possess brain specificity. Pep-9, as the peptide “Trojan horse”, was chosen to ferry IgG molecules into the brain, which was investigated in mice model. IgG-Pep-9 conjugate formulation was developed by click chemistry, delivered intravenously in mice and allowed to circulate for 24 h before brain tissue harvesting. The results indicated that Pep-9 enhanced IgG delivery into the brain parenchyma. These findings prove that Pep-9 can carry the IgG molecule to transport BBB and deliver into brain parenchyma.

The proven ability of these peptides to ferry small molecules (e.g., fluorophore) and large macromolecules such as phage highlights their potential to effectively shuttle different nanomedicines into the brain to diagnose and treat CNS diseases. These and other aspects of the disclosure are described in detail below.

### I. Blood-Brain Barrier

The BBB consists of brain capillary endothelial cells and is regulated by supporting cells, such as pericytes, astrocytes, and other glial cells, to form a tight and continuous barrier⁶. During homeostasis, the polarized brain capillary endothelium simultaneously protects the brain from exposure to exogenous or toxic solutes and mediates the selective exchange of essential nutrients, ions, and metabolites between blood and the brain interstitium by diffusion, transporters, and adsorptive- and receptor-mediated transport⁶⁻⁸. Typically, drugs exploit these pathways to permeate the BBB via the following: (1) hydrophobic small molecule drugs (<400 Da) diffuse and penetrate through the endothelium; (2) other small molecules shuttle across the BBB by paracellular flux (i.e., openings between the endothelium); and (3) macromolecules including peptides and proteins use endogenous transport mechanisms (e.g., transferrin and insulin receptors, electrostatic adsorption) to actively transcytose, or go across, the BBB and enter the brain parenchyma⁹⁻¹². In spite of extensive efforts, drug delivery into the brain has not been successful, with ˜98% of all small molecule drugs and nearly 100% of all biologics unable to shuttle across the BBB and reach the brain parenchyma¹³.

After traversing the BBB, drugs and drug carriers must also navigate through the ubiquitous but underexplored extracellular space (ECS) prior to reaching the target cells in the brain. The ECS is a fluid-filled space (“water phase of a foam”) that surrounds all cells of the CNS and occupies 20% of the total brain volume¹⁴. The ECS maintains the dynamic flow of interstitial fluids¹⁵⁻¹⁷ and the ionic balance across the cell membranes^{18,19}. The ECS has an irregular structure around the cells with microdomains of void spaces²⁰. It consists of negatively charged, highly condensed extracellular matrix including high amounts of glycosaminoglycans (e.g., hyaluronan and heparin sulfate), proteoglycans¹⁶ and fibrous proteins (e.g., collagen and fibronectin). The geometry and composition of the ECS combine to hinder diffusive transport of molecules and drug delivery to the brain parenchyma^{17,21}. In the ECS, antibodies have been shown to bind to receptors, and lactoferrin¹⁸ binds to negatively charged heparin sulfate of the extracellular matrix²²; these molecules demonstrate significantly decreased transport in the ECS than in free medium. Consequently, solutes such as drugs need to circumvent size filtration and intermolecular interactions with the mesh-like network of brain extracellular matrix to diffuse through the ECS and reach target cells^{23,24}.

### II. Peptides

As defined herein, a peptide is a short series of amino acids connected by peptide bonds. The amino acids may be naturally-occurring amino acids or may be synthetic/non-natural amino acids. The peptides will in general be around 25 residues in length. Thus, the term “a peptide having no more than 25 consecutive residues,” even when including the term “comprising,” cannot be understood to comprise a greater number of consecutive amino acid residues. The overall length may be 7, 8, 9, 10, 11, 12, 13, 14, 15, 16, 17, 18, 19, 20, 21, 22, 23, 24, or residues. Ranges of peptide length of 7-25 residues, 8-25 residues, 9-25 residues, 10-25 residues, 12-25 residues, 15-25 residues, 20-25, residues, 7-20 residues, 8-20 residues, 9-20 residues, 10-20 residues, 12-20 residues, 15-20 residues, 7-15 residues, 8-15 residues, 9-15 residues, 10-15 residues and 12-15 residues are contemplated.

The peptides may utilize L-configuration amino acids, D-configuration amino acids, or a mixture thereof. While L-amino acids represent the vast majority of amino acids found in proteins, D-amino acids are found in some proteins produced by exotic sea-dwelling organisms, such as cone snails. They are also abundant components of the peptidoglycan cell walls of bacteria. D-serine may act as a neurotransmitter in the brain. The L and D convention for amino acid configuration refers not to the optical activity of the amino acid itself, but rather to the optical activity of the isomer of glyceraldehyde from which that amino acid can theoretically be synthesized (D-glyceraldehyde is dextrorotary; L-glyceraldehyde is levorotary).

One form of an “all-D” peptide is a retro-inverso peptide. Retro-inverso modification of naturally-occurring polypeptides involves the synthetic assemblage of amino acids with α-carbon stereochemistry opposite to that of the corresponding L-amino acids, i.e., D-amino acids in reverse order with respect to the native peptide sequence. A retro-inverso analogue thus has reversed termini and reversed direction of peptide bonds (NH—CO rather than CO—NH) while approximately maintaining the topology of the side chains as in the native peptide sequence. See U.S. Pat. No. 6,261,569, incorporated herein by reference.

It will be advantageous to produce peptides using the solid-phase synthetic techniques (Merrifield, 1963). Other peptide synthesis techniques are well known to those of skill in the art (Bodanszky et al., 1976; Peptide Synthesis, 1985; Solid Phase Peptide Synthelia, 1984). Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

Aside from the 20 standard amino acids can be used, there are a vast number of “non-standard” or “non-natural” amino acids. Two of these can be specified by the genetic code but are rather rare in proteins. Selenocysteine is incorporated into some proteins at a UGA codon, which is normally a stop codon. Pyrrolysine is used by some methanogenic archaea in enzymes that they use to produce methane. It is coded for with the codon UAG. Examples of non-standard amino acids that are not found in proteins include lanthionine, 2-aminoisobutyric acid, dehydroalanine and the neurotransmitter gamma-aminobutyric acid. Non-standard amino acids often occur as intermediates in the metabolic pathways for standard amino acids—for example ornithine and citrulline occur in the urea cycle, part of amino acid catabolism. Non-standard amino acids are usually formed through modifications to standard amino acids. For example, homocysteine is formed through the transsulfuration pathway or by the demethylation of methionine via the intermediate metabolite S-adenosyl methionine, while hydroxyproline is made by a posttranslational modification of proline.

### III. Formulation and Administration

The present disclosure provides pharmaceutical compositions comprising peptides and peptide conjugates. Such compositions comprise a diagnostically, prophylactically or therapeutically effective amount of a peptide/peptide conjugate and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the peptide or conjugate, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### IV. Peptide Conjugation

### A. Conjugation Chemistries

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

Crosslinking methods that may be used include, e.g., radiation, dehydrothermal heat treatment, and chemical crosslinking. Chemical crosslinking agents may be used to crosslink proteins using, e.g., carboxyl, carbonyl, sulfhydryl, amine or hydroxyl reactive agents. Homo bi (or poly) functional or hetero bi (or poly) functional agents can be used for crosslinking. In addition, enzymes can also be used for crosslinking. Common agents that may be used to promote crosslinking include, e.g., glutaraldehyde, di succinimide esters of N-hydroxy succinimide (NHS), such as polyethylene glycol NHS esters, carbo-diimide crosslinkers, maleimides, imidoesters, haloacetyls, pyridyl disulfides, hydrazides, glyoxals, sulfones, periodates, isocynates, ureas, disulfides. Activatable crosslinkers, such as photoactivated crosslinkers, can also be used including psoralens, aryl azides or diazirines. Radiation and dehydrothermal treatement may be preferably used in some embodiments, as they offer the benefit of not needing to introduce new chemical agents into the films.

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected peptide or conjugate) and through the thiol reactive group, the cross-linker, already tied up to the first protein, reacts with the cysteine residue (free sulfhydryl group) of the other protein (e.g., the selective agent).

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to an antibody (U.S. Pat. Nos. 4,472,509 and 4,938,948).

Proteins may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies where the detectable imaging moieties are bound to the antibody using linkers such as methyl-p-hydroxybenzimidate or N-succinimidyl-3-(4-hydroxyphenyl)propionate.

### B. Diagnostic Conjugation Partners

Peptides of the present disclosure may be linked to at least one agent to form a conjugate. In order to increase the efficacy of peptides as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to peptides include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to peptides include enzymes, radiolabels, haptens, fluorescent labels, phosphorescent molecules, chemiluminescent molecules, chromophores, photoaffinity molecules, colored particles or ligands, such as biotin.

Peptide conjugates are useful as diagnostic agents. Many appropriate imaging agents are known in the art, as are methods for their attachment to proteins (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatin²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium⁹⁰, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Intermediary functional groups are often used to bind radioisotopes which exist as metallic ions, such as diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6 G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional conjugates would include a binding ligand and/or an enzyme (an enzyme tag) that will generate a colored product upon contact with a chromogenic substrate. Examples of suitable enzymes include urease, alkaline phosphatase, (horseradish) hydrogen peroxidase or glucose oxidase. Particular binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as binding agents.

### C. Therapeutic Conjugation Partners

A wide variety of therapeutic agents may be conjugated to the peptides of the present disclosure. Exemplary agents are discussed below.

Chemotherapeutic agents are commonly used to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γl and calicheamicin ω1; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

Radioisotopes, while useful as diagnostic reagents, are also use therapeutically. Radiotherapeutic isotopes include, but are not limited to, Ac-225, Ac-227, At-211, Au-198, B-11, Bi-212, Bi-213, Cf-252, Co-60, Cs-131, Cu-67, I-125, I-131, Ir-192, Os-194, P-32, Pd-103, Pd-109, Ra-223, Re-186, Re-188, Rh-105, Sc-47, Si-28, Sm-145, Sn-117m, Ta-182, Tb-149, Th-228, Tm-170, W-188, Y-90, and Y-91.

Antibiotics are drugs which may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

In some embodiments, the peptides of the present disclosure can be conjugated to an antibiotic. Antibiotics can fall into a wide range of classes, such as narrow spectrum antibiotics that target a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the peptides could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

Additional agents suitable for conjugation with the peptides disclosed herein include neuroregenerative agents, neuroprotective agents, neurotrophic factors, growth factors, cytokines, chemokines, antibodies, immunosuppressive agents, steroids, anti-fungals, anti-virals or other agents. In even more particular embodiments, the neuroprotective agent is for example dopamine D3 receptor agonists, the neurotrophic factors are for example BDNF, NT-3, NT-4, CNTF, NGF, or GDNF; the antibodies are for example IN-I anti-NOGO antibodies; the immunosuppressive agents are for example corticosteroids, cyclosporine, tacrolimus, sirolimus, methotrexate, azathiopine, mercatopurine, antibodies such as anti-T-cell receptor (CD23) and anti-IL2 receptor (CD25) antibodies, interferon, opioids, TNF binding proteins, mycophenolate, and small biological agents such as FTY720; the steroid is methylprednisolone.

### V. Kits

In still further embodiments, the present disclosure concerns kits for use with the peptides and peptide conjugates described above. As the peptides and conjugates may be used to detect or treat diseases or disorders and thus may be included in kit form. The kits will thus comprise, in suitable container means, a first peptide and optionally a detection reagent or a therapeutic agent.

The reagents of the kit may take any one of a variety of forms, including those detectable labels or therapeutic agents are pre-associated with or linked to the given peptide. Alternatively, the labels/agents and the peptides may be provided separately and the kit may optionally contain reagents for conjugating the labels/agents with the peptides. The components of the kits may be packaged either in aqueous media or in lyophilized form.

The container means of the kits will generally include at least one vial, test tube, flask, bottle, syringe or other container means, into which the peptides/labels/agents may be placed, or preferably, suitably aliquoted. The kits of the present disclosure will also typically include a means for containing the reagent containers in close confinement for commercial sale. Such containers may include injection or blow-molded plastic containers into which the desired vials are retained.

### VI. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

CX7C-presenting M13 phage biopanning against hCMEC/D3 cells. A M13 phage CX7C library was used to pan against established BBB model hCMEC/D3 in vitro to identify potential brain-penetrating peptides. hCMEC/D3 cells (CELLutions Biosystems Inc.) were cultured in EndoGRO medium (Millipore, #SCME004) on Collagen I (Fisher Scientific, #44310001) coated Culture Ware (150 μg/ml, coated for at least 1 h at 37° C.)¹¹⁰. Besides collagen I coating on cell culture surface, hCMEC/D3 cells produce extracellular matrix components for adhesion and support and can partially provide a mimic of the brain ECS. To establish the cell monolayer on the transwell plate setup, hCMEC/D3 cells (passage 27-30) were seeded on a pre-coated collagen I (100 μg/ml for 3 h) coated 12-well transwell plate (Corning, #3401) at a density of 1.5-2.0*10⁵ cells/cm² for 12-15 days⁴⁹. Transepithelial electrical resistance measurements and permeability assay with small tracer molecule sodium fluorescein were used to monitor the tightness of hCMEC/D3 monolayer. Prior to biopanning, hCMEC/D3 cells were incubated in fetal bovine serum depleted medium for 1 h at 37° C., and then 10¹¹ plaque forming units (pfu) of M13 phage CX7C library (NEB, #E8120S) was added to the donor compartment of the transwell and incubated for 1 h at 37° C. After the “pulse”, the donor and receiver compartment volumes were collected, the surface of hCMEC/D3 cells was washed for 3 times with phosphate buffered saline (PBS). The transwell inserts were then transferred to a new 12-well plate with replenished fresh medium and incubated for 1 h at 37° C. After, the eluate from receiver compartment was collected and amplified in XL-1 Blue E. coli (Fisher scientific #50-125-053) for the subsequent round of biopanning. In total, the biopanning was done for three rounds, and each round was done in duplicate. The phage library DNA from the amplified eluates were isolated and prepared for next generation sequencing (NGS) (see below). In addition, for analysis of fast-growing phage, the original, naïve M13 CX7C library was also amplified in XL-1 Blue E. coli for three rounds without any selection and the pooled library DNA was prepared for NGS.

Phage DNA sample preparation and next generation sequencing (NGS) analysis. Twenty microliters (phage concentration ˜1.0*10⁸pfu/μL, which is equivalent to phage DNA concentration 5 ng/μL) of each amplified eluate from biopanning and naive library were incubated at 100° C. for 15 min then cooled down at room temperature. Library DNA preparation protocol (Illumina 16 S metagenomic sequencing library protocol) was followed according to the manufacturer's recommendations to prepare the M13 phage library DNA. Briefly two-step PCR was performed to prepare the library DNA amplicon. The first step PCR was to amplify the random region of the M13 library DNA. The primers were designed as 5′ TCG TCG GCA GCG TCA GAT GTG TAT AAG AGA CAG AGC AAG CTG ATA AAC CGA TAC A 3′ (forward primer; SEQ ID NO: 21) and 5′ GTC TCG TGG GCT CGG AGA TGT GTA TAA GAG ACA GTT GTC GTC TTT CCA GAC GTT AG 3′ (reverse primer; SEQ ID NO: 22). The second step PCR was to add the barcodes to the first step PCR product. Here for the second step PCR, the PCR primers were provided with Nextera XT Index kit (FC-131-1001, Illumina). Two steps of PCR sample preparation, including PCR conditions, PCR product clean up, and library DNA pooling were performed according to the instructions provided in the protocol (16 S-, Illumina). Two replicates of eluates from every round of biopanning were prepared for the library DNA and pooled to run by Illumina MiSeq on two different batches.

Bioinformatics analysis of the top 20 peptide sequences. The NGS data from two replicates of MiSeq run were analyzed on Stampede, a supercomputer run on Texas Advanced Computing Center at UT-Austin. First, to confirm the sequence quality and trim the low-quality sequence reads, fastqc and fastx_toolkit were used on the dataset. Customized Perl and Bash scripts were written to filter out sequences of the insertless M13 phage DNA. Customized Python and bash scripts were used to translate DNA sequences into CX7C peptides and count the distribution (i.e., frequency) of CX7C peptides. Meanwhile, additional online tools (Clustal Omega, Emboss transeq, and Gibbs Cluster server) were also used to confirm the quality of DNA sequence reads, translate DNA sequences to peptide sequences, as well as calculate the motifs shown in each round of biopanning. After filtering the low-quality and insertless M13 phage DNA sequences, the 20 most frequent CX7C peptides from the third round of biopanning were selected for further enrichment analysis. The motifs were calculated from the 20 most abundant peptide sequences from each round of biopanning and the amplification of M13 phage CX7C naive library. Physiochemical properties were also calculated to the 20 most frequent peptides¹¹¹. Protein Calculator v3.4 (The Scripps Research Institute) was used to calculate the net charge and grand average of hydropathy (GRAVY), where the more positive score is indicative of greater hydrophobicity of the sequence.

Cloning CX7C peptide-presenting M13 phage and their scrambled controls. From the enrichment analysis of the 20 most frequent peptides from the third round of panning, the top 11 frequent peptides showed significant enrichment from the three rounds biopanning. Therefore, complementary DNA oligonucleotides were designed for the 11 most frequent peptides and their respective scrambled controls. Meanwhile, oligonucleotides were also designed for the motif sequences shown in 20 most frequent peptides in the third round of screening. Oligonucleotides were also designed encoding for positive control BBB shuttle peptides THRPPMWSPVWP (SEQ ID NO: 23) and CRTIGPSVC (SEQ ID NO: 24). One of the non-enriched peptide NC was selected as the negative control, and oligonucleotides encoding for the peptide were designed for this control. DNA oligonucleotides (synthesized by IDT) were dissolved in DNase free and RNase free H₂O to a stock concentration of 100 μM and subsequently diluted to 1 μM as the working concentration. Complimentary oligonucleotides encoding for the respective peptides were annealed starting at 95° C. for 2 min and cooled down to room temperature in 1 h using a heat-block plate (ThermoFisher Scientific, model #2001).

Annealed oligonucleotides were phosphorylated using T4 polynucleotide kinase (NEB) following the manufacturer's recommendations. M13KE phage cloning vector (NEB) was double-digested with Kpn I and Eag I high fidelity restriction enzymes (NEB) at room temperature for 2 h. Double digested M13KE phage vector was ligated with phosphorylated annealed oligos encoding for the top 11 peptides and the scrambled control sequences at 16° C. overnight with T4 ligase (NEB). XL-1 Blue chemically competent cells (Fisher Scientific #50-125-053) were transformed with ligated DNA by the heat shock transformation and overlaid on solid agar plates for plaque formation. The resulting phage plaques were isolated, and phage DNA was isolated and purified. Sanger sequencing confirmed the identity and correct insertion of the peptide sequence in pIII region of M13. The correctly sequenced peptide-presenting phage clones were amplified in XL-1 Blue E. coli in sufficient quantities for subsequent in vitro and in vivo experiments.

Transcytosis of CX7C peptide presenting M13 phage against hCMEC/D3 cells. hCMEC/D3 cells were seeded on the 12-well transwell plate at passage 27-30 following the same procedures described in biopanning. Approximately 10⁹-10¹⁰ pfu of each M13 clone (input) was incubated with hCMEC/D3 cells. The eluate output in the receiver compartment of the transwell system from second-hour incubation after “pulse” assay was collected and titered by standard double-layer plaque assay. The total titer of phage (pfu) in the eluate was calculated for each M13 clone. The ratio of output to input phage (i.e., ratio of phage clone that transcytoses the BBB model) was calculated to obtain the efficiency of BBB shuttling for each M13 clone and control, and each clone was run in triplicate. To compare the sequence specificity of M13 clones, selected BBB-shuttling clones and their respective scrambled controls were run using the same transcytosis assay as described earlier. In addition, temperature-dependent transcytosis assays were run using the same setup at 4° C. and 37° C.

Intracellular tracking of fluorescently labeled M13 phage. Select peptide-presenting M13 phage (Pep-1, Pep-2, Pep-3, Pep-4, Pep-5, Pep-8, and Pep-9), motif-sequence clone, negative and positive control M13 clones were conjugated with Alexa Fluor 488 5-sulfodicholorphenol (SDP)-ester, (ThermoFisher Scientific, #A30052) at a ratio of 1.0*10¹⁰-10¹¹ pfu/50 μg dye. The fluorescent dye labeling was done shaking on a rocker for 1 h at room temperature. The labeling reactions were then dialyzed overnight with a dialysis cassette with a molecular weight cutoff of 3500-5000 Da (Spectrum Labs, #G235029, MWCO: 3.5 k-5 k). Dialyzed M13 clones were precipitated with ⅙ volume of 20% polyethylene glycol (PEG) and 2.5 M NaCl overnight at 4° C. After centrifugation, the resulting phage pellets were reconstituted in PBS. For tracking studies, hCMEC/D3 cells were cultured in 8-well chamber slide (ThermoFisher Scientific #154534) at a seeding density of 1.0-2.0×10⁴ cells/cm². For each clone, 10⁸-10⁹ pfu of each fluorescent-labeled M13 phage was incubated with confluent hCMEC/D3 cells for 1 h at 37° C. Then, the cell culture medium with unbound phage clones was removed, cells were washed with PBS three times, and fresh cell culture medium was added to the chamber prior to 2D particle tracking.

Wide-field imaging for single-particle tracking (SPT) was performed using an Olympus IX71 inverted microscope equipped with a 60×1.2 N.A. water objective (UPLSAPO 60XW, Olympus). All imaging was conducted at 37° C. using a temperature-controlled stage (Stable Z System, Bioptechs). Wide-field excitation was provided by a metal halide lamp with a 480/40 nm BP excitation filter. Emission was collected by a Scientific CMOS camera (ORCA-Flash4.0) through 510 nm LP dichroic mirror and 535/50 BP filter. The pixel size is equivalent to 107 nm. Fluorescent images of labeled phage were acquired at 20 frames per second for a total of 600 frames. The analysis of the acquired image series was performed as described previously^{112,113} to obtain trajectories. The SPT software was a gift from Prof. Keith Lidke at the University of New Mexico. The trajectories were analyzed using a mean-squared displacement (MSD)-based trajectory classification algorithm⁶⁴ to extract the diffusion coefficient (D) and identify the sub-trajectories exhibiting active transport.

Cellular uptake of M13 clones in hCMEC/D3 cells. hCMEC/D3 cells (passage 27-30) were seeded in a 12-well plate (Corning #3513) at a seeding density of 4.0*10⁴/cm² and cultured for 5 days. Each M13 clone (˜10⁹ pfu) was added to the confluent hCMEC/D3 cells and incubated at 37° C. for 1 h. Then, the culture medium was removed, stripping buffer (0.2% BSA in the basal endothelial culture medium, pH 3.5 adjusted by HCl) was added to remove cell-surface bound M13 phage¹¹⁴. hCMEC/D3 cells were then washed with PBS three times, and cells were lysed with RIPA buffer (ThermoFisher Scientific, #89900) to collect M13 clones internalized in hCMEC/D3 cells. M13 phage from cell lysate was quantified by double-layer agar plaque assay.

Transcytosis of CX7C peptides against hCMEC/D3. Selected CX7C peptides and controls were synthesized and fluorescently labeled with 5′carboxyfluorescein (5-FAM) at the N-terminus of each peptide (Lifetein, LLC). Transcytosis of peptides was performed against confluent hCMEC/D3 cells cultured in the 24-well transwell plate (Corning #3413) as described earlier. Upon formation of tight, continuous hCMEC/D3 cell monolayer on day 12-15, cells were incubated in fetal bovine serum-free medium for 1 h. Then 100 μM of each peptide (concentration dependent assay determined by flow cytometry, data not shown) was prepared in 100 μL basal culture medium and added to donor compartment (total 10 nmol for each FAM-peptide), and 600 μL medium was replenished the receiver compartment. At 5, 10, 15, 30, 45, 60, 90 and 120 mins, each transwell insert was temporarily transferred to the neighboring empty wells, and the fluorescence intensity in the receiver compartment (denoted as output) was read using a plate reader (Infinite M200, Tecan)¹¹⁵. The input of each peptide was the fluorescence intensity of ten nmol FAM-peptide that was diluted and measured 600 μL basal medium in the receiver compartment. The ratio of output to input was calculated to compare the transcytosis efficiency of the CX7C and controls. To compare the energy-dependent effect of transcytosis, the assay was run at 37° C. and 4° C. for each peptide.

Diffusion of selected peptides through Matrigel in a transwell assay. Matrigel (Corning #354230) was used as an in vitro mimic of the ECM with 2 mm thickness coating in 24-well transwell insert¹¹⁶. Matrigel consists of soluble basement membrane proteins derived from the Engelbreth-Holm-Swarm mouse sarcoma, a tumor rich in ECM components^{117,118} Matrigel-formed ECM barrier has a pore size around 0.14 μm⁸⁹, which has been frequently used for diffusion study of solutes including nanoparticles⁸⁷⁻⁸⁹. Matrigel was added to the transwell and allowed to gel at 37° C. for 30 min. Upon gelation, 200 μL of 100 μM CX7C and control peptides (total 20 nmol for each peptide) were added to the donor compartment, 1000 μL fresh medium was replenished to the receiver compartment to maintain sink conditions. The fluorescence intensity was measured in the receiver compartment using the plate reader every 5 min for the first 1 h and every 1 h up to 6 h¹¹⁵. The output to input (ratio of fluorescence in the receiver compartment at each time point (output) to the fluorescence of the initial 20 nmol each peptide diluted in 1000 μL fresh medium in a new receiver compartment of the transwell plate (input)) was calculated to compare the diffusion transport of each peptide in Matrigel.

Diffusion of selected peptides through Matrigel embedded in a multi-channel chamber slide. For diffusion through the microchannel p-Slide 0.4 VI chamber (ibidi), each channel was loaded with 30 μL Matrigel and was allowed to gel in a cold room following the manufacturer's recommendations. After gelation, the basal medium was added to hydrate the gel for 10 min. 60 μL of 100 μM of each peptide was added to the inlet reservoir of the chamber, and 60 μL of the basal medium was added to the other reservoir to create sink conditions. Olympus IX83 fluorescence microscope was set-up to image migration of fluorescent peptides in the Matrigel filled channel by recording fluorescent images every 30 min up to 12 h with Hamamatsu ORCA-flash 4.0 camera and 4*UPLSAPO objective using the GFP filter. Custom MATLAB scripts were written to analyze the fluorescent images (see Table A)⁹⁰. The following equation was used to calculate the diffusion coefficient of each peptide in the Matrigel⁹²:

$$C ( x , t ) α ( \frac{x}{2 \sqrt{{D}_{eff} t}} ) .$$

Brain distribution of Pep-3, Pep-9 and negative control phage clones and M13 conjugated with biotinylated Pep-3, Pep-9 and Angiopep-2 in vivo. An in vivo study to determine the accumulation of select M13 phage clones in the brain was performed in accordance with an animal protocol approved by the IACUC committee at The University of Texas at Austin. Six to eight weeks old female Balb/c mice were injected by tail vein injection with either 100 μL of Pep-3, Pep-9, or negative control NC phage clones at a concentration ˜4.8−6.3×10⁷ pfu/μL (n=5 for each group). Phage were genetically engineered to display Pep-3, Pep-9, or negative control as described above. After injection, phage were allowed to circulate for 30 min.

For in vivo brain uptake of phage conjugated with biotinylated Pep-3, Pep-9, or Angiopep-2, the inventors did the following. Oligonucleotides encoding for the biotin acceptor peptide (BAP, sequence: GLNDIFEAQKIEWHE⁹⁷ (SEQ ID NO: 25)) was genetically engineered into the M13KE display vector at the cut sites by Kpn I and Eag I restriction enzymes, and the ligated DNA was transformed, and the phage product was propagated as described earlier. Free peptides Pep-3, Pep-9 and Angiopep-2 with N-terminal biotin modification were synthesized with purity>95% by solid-phase Fmoc chemistry (Lifetein, LLC). In separate reactions, BirA biotin-protein ligase (Cat. #BirA500, Avidity, LLC) was added to enzymatically conjugate biotinylated Pep-3, Pep-9, and Angiopep-2 peptide to BAP displayed on pIII of M13 phage⁹⁸. These phage were quantified by standard double-layer agarose plaque assay. Six to eight weeks old female Balb/c mice were injected by tail vein injection with 10⁹ pfu of these phage conjugated with biotinylated peptides (˜150 μL), and phage were allowed to circulate for 30 min.

Thirty minutes post-injection, mice were humanely euthanized by CO2 inhalation, and 300-400 μL blood was collected by cardiac puncture and stored in heparin blood collection tube (BD #365985). Mice were then perfused via transcardiac perfusion¹¹⁹ with a syringe pump at a flow rate of 2.5 mL/min, for a total of 10 ml PBS for each mouse. Brains were dissected and stored in 2 ml ice-cold physiological buffer¹²⁰ with complete protease inhibitor cocktail (Roche, #5892791001)¹²¹. To isolate the brain parenchyma from the brain capillary, capillary depletion was performed following the method developed by Triguero et al.¹²⁰. Briefly, harvested brains were homogenized and dextran solution was added to the brain homogenate to obtain a final 13% dextran concentration. Then, the brain parenchyma supernatant was separated from the brain capillary pellet using dextran density centrifugation at 5400 g at 4° C. for 15 min¹²⁰. The blood, brain supernatant, and capillary samples after perfusion and capillary depletion were stored at −80° C. until use. The amount of Pep-3, Pep-9, and negative control-presenting M13 phage (and M13 phage conjugated with Pep-3, Pep-9, and Angiopep-2) in the blood, brain parenchyma supernatant, and blood vessel (i.e., capillary) were quantified by double-layer plaque assay¹²². From these values, the ratio of phage in brain parenchyma/blood serum (in μL/g) and brain capillary/blood serum (in μL/g) were calculated to compare the BBB shuttling efficiency and brain accumulation of M13 clones.

Biodistribution of fluorescently labeled Pep-3, Pep-9 and Angiopep-2 peptides in vivo. Six to eight weeks old female Balb/c mice (20-23 g) were injected by tail vein injection with either 100 μL of 500 μM FAM-labeled Pep-3, Pep-9, or Angiopep-2 (diluted in PBS buffer) and allowed to circulate for 30 min¹⁰³. After, 20 ml PBS buffer was run at 3 ml/min with an infusion pump to flush all the organs by transcardial perfusion. Before tissue harvesting, 10 ml of 4% formaldehyde was perfused to fixate all the tissues. The brain, lung, heart, kidney, and liver tissue were harvested from all mice. The IVIS Spectrum in vivo imaging system was set up to take the fluorescent images of all the tissues (excitation/emission wavelength 500 nm/540 nm). Manual regions of interest (ROI) were drawn around each organ in all the mice, the total radiant efficiency was calculated to compare the biodistribution of each peptide, data were analyzed by multiple t-tests with P<0.05.

### Example 2—Results and Discussion

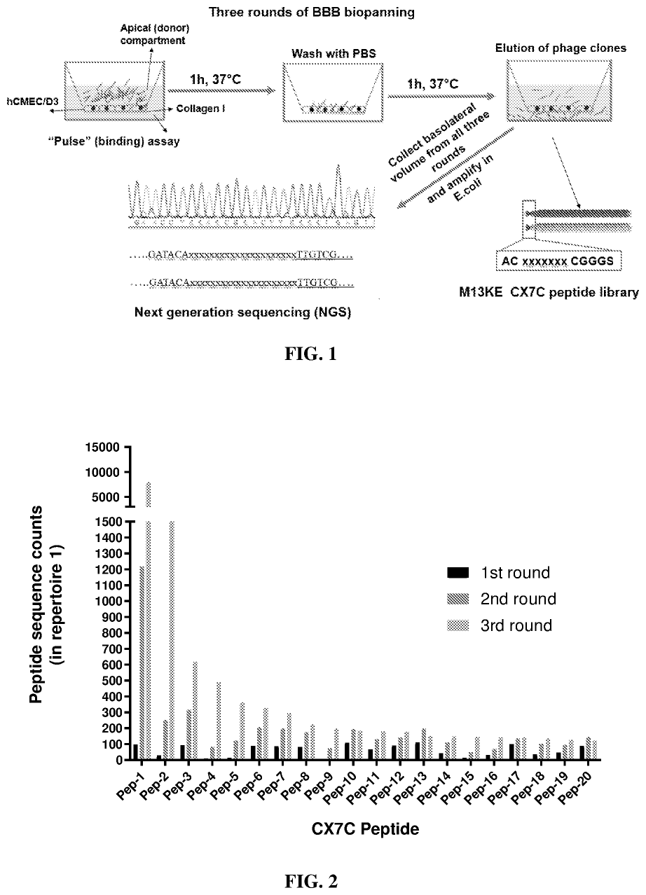

Identification of BBB penetrating peptides by next-generation sequencing and analysis of their physicochemical properties. Adapting from the pulse-chase assays used to study transcytosis of TfR antibody across the BBB^{49,50}, the inventors developed a modified “pulse”-only assay to pan M13 phage libraries in vitro against a human-derived BBB cell line hCMEC/D3 through iterative screening to select for phage clones that transport across the BBB model and the underlying collagen matrix in a transwell system (FIG. 1). hCMEC/D3 is a well-established in vitro BBB model used in drug transport studies that recapitulates the phenotype of the human BBB and avoids potential species differences from in vitro and in vivo rodent models⁵¹. Cysteine-constrained cyclic peptide libraries were used because they have more conformational rigidity to bind to targets with high affinity, are stable, and less susceptible to protease degradation⁵². The eluates of phage clones from each round were collected and amplified, and then they were either added to hCMEC/D3 for the subsequent round of panning, or their DNA was prepared for next-generation DNA sequencing (NGS). Since the M13 genome encodes for its phenotype, the phage-displayed peptide sequences can be identified by DNA sequencing. Isolated phage DNA was analyzed by NGS to obtain a larger number of DNA sequences (i.e., the number of reads) than Sanger sequencing. Traditionally, the number of phage clones identified by Sanger sequencing is limited to 5-1000; using NGS, it is feasible to obtain up to around 2.5×10⁷ reads by the Illumina Miseq platform, which allows for sufficient sampling of the CX7C library from biopanning and control experiments⁵³. After, NGS data was analyzed to identify peptide sequences and their frequency from each round of panning.

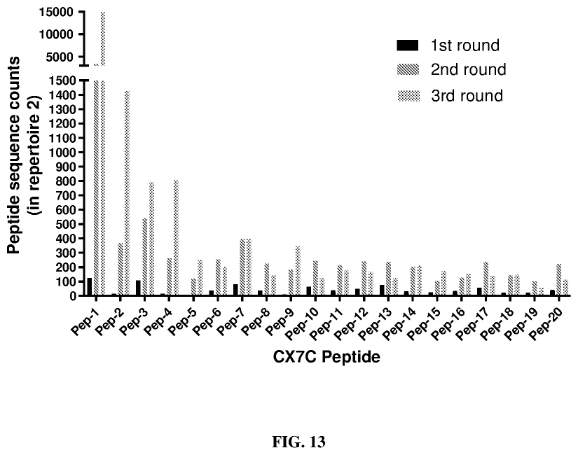

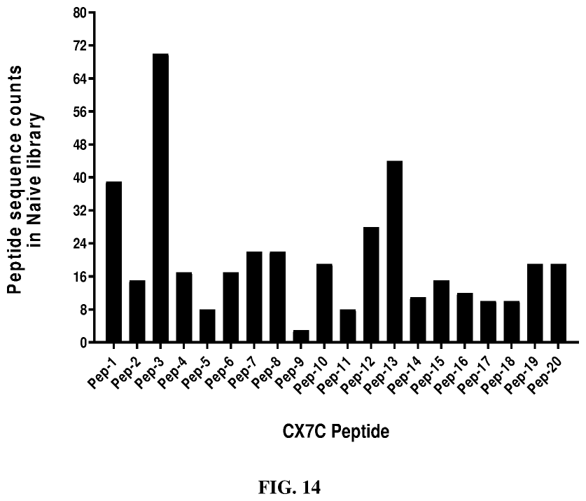

From NGS, DNA sequences were identified and translated into peptides from each round of biopanning in each replicate. After excluding the insertless M13 phage from NGS dataset, the 20 most abundant peptides from the third round of biopanning were identified, and their frequency in the first two rounds was also determined from the NGS dataset (FIG. 2 and FIG. 13). In the third round of biopanning, both replicates shared 18 out of 20 of the most frequent sequences. The dominant peptide from the third round was Pep-1 with 7961 counts, which was approximately five-fold higher than the second most frequent peptide Pep-2. The frequency of remaining sequences (Table 1) ranged from 121-618 counts in the third round of biopanning. In both replicates, the eleven most abundant sequences (Pep-1 to Pep-11) exhibited apparent enrichment between successive rounds of biopanning (FIGS. 2 and 13). With each successive round of panning, the increased frequency of the peptide is indicative of their affinity for the target⁵⁴. To ensure that the abundant sequences were due to selection enrichment and not because of their growth bias in bacteria^{53,55}, the naïve, original library was amplified in E. coli for three successive rounds without selection pressure, and the library DNA was sequenced by NGS. The twenty most frequent sequences from selection were not abundant in the third round of amplified naïve library; the counts ranged from 3-70 (FIG. 14). This additional filter is needed to exclude potential false positives due to fast-growing phage clones⁵⁶. During biopanning, phage clones are selected against the target and subsequently these clones are amplified in host bacteria to make more copies; however, this results in phage clones that have high affinity for the target and/or phage clones that easily amplify in bacteria. It has been demonstrated that amplification-based selection (i.e., clones that grow faster than other clones) is independent of target-based selection^{44,55-57}. Even without target-based selection, the diversity of phage libraries can collapse due to amplification-based selection. These findings indicate that the twenty most frequent peptide-presenting phage clones from selection are not parasitic clones, and instead, their increased frequency with successive rounds of biopanning suggests that they may bind to targets on hCMEC/D3 cells.

The physiochemical properties of the twenty most abundant peptides (Pep-1 to Pep-20) were determined in silico (Table 1). Here, 12 were basic, 1 was acidic, and 7 possessed a net neutral charge. The grand average of hydropathy (GRAVY) score was calculated as a value of the hydrophobicity (or hydrophilicity) of the peptides⁵⁸; the more positive GRAVY score correlates with greater hydrophobicity of the peptide sequence. From the listed sequences, 11 of them were hydrophobic (Table 1).

TABLE 1
Physiochemical properties of 20 most frequent
peptides in the BBB biopanning (sequences as tested also
include cysteine at both ends resulting in cyclization)

| | Peptide | | | | | Hydropathy |
|---|---|---|---|---|---|---|
| Peptide | sequence | Charge | Net | | m.w. | (Gravy |
| name | (SEQ ID NO: X) | Attribute | charge | PI | (g/mol) | score) |
| Pep-1 | NAGHLSQ | basic | 1 | 6.89 | 932.04 | -0.089 |
| | (SEQ ID NO: 1) | | | | | |
| Pep-2 | SAYDRPL | neutral | 0 | 5.85 | 1027.2 | -0.122 |
| | (SEQ ID NO: 2) | | | | | |
| Pep-3 | NSHTQGK | basic | 2 | 8.07 | 911.12 | -1.222 |
| | (SEQ ID NO: 3) | | | | | |
| Pep-4 | TYLNSAK | basic | 1 | 7.98 | 1002.2 | 0.044 |
| | (SEQ ID NO: 4) | | | | | |
| Pep-5 | VNQGSIG | neutral | 0 | 5.02 | 880.05 | 0.567 |
| | (SEQ ID NO: 5) | | | | | |
| Pep-6 | NIKSSHV | basic | 2 | 8.07 | 990.2 | 0.167 |
| | (SEQ ID NO: 6) | | | | | |
| Pep-7 | VPSKPGL | basic | 1 | 7.99 | 903.16 | 0.522 |
| | (SEQ ID NO: 7) | | | | | |
| Pep-8 | NWMINKE | neutral | 0 | 5.93 | 1140.4 | -0.433 |
| | (SEQ ID NO: 8) | | | | | |
| Pep-9 | LWRPAAD | neutral | 0 | 5.85 | 1034.24 | 0.211 |
| | (SEQ ID NO: 9) | | | | | |
| Pep-10 | CSKEATPFC | neutral | 0 | 5.93 | 985.16 | -0.100 |
| | (SEQ ID NO: 10) | | | | | |
| Pep-11 | SSKHEAT | basic | 1 | 6.89 | 965.09 | -0.678 |
| | (SEQ ID NO: 11) | | | | | |
| Pep-12 | IHSPTAL | basic | 1 | 6.89 | 944.17 | 0.978 |
| | (SEQ ID NO: 12) | | | | | |
| Pep-13 | LTAKHMQ | basic | 2 | 8 | 1034.32 | 0.133 |
| | (SEQ ID NO: 13) | | | | | |
| Pep-14 | GPTAKYI | basic | 1 | 7.98 | 955.19 | 0.378 |
| | (SEQ ID NO: 14) | | | | | |
| Pep-15 | DGLAKNS | neutral | 0 | 5.85 | 910.06 | -0.167 |
| | (SEQ ID NO: 15) | | | | | |
| Pep-16 | ISSSINH | basic | 1 | 6.89 | 963.13 | 0.544 |
| | (SEQ ID NO: 16) | | | | | |
| Pep-17 | NMHTPMV | basic | 1 | 6.89 | 1035.32 | 0.444 |
| | (SEQ ID NO: 17) | | | | | |
| Pep-18 | TTKLPNS | basic | 1 | 7.99 | 966.17 | -0.267 |
| | (SEQ ID NO: 18) | | | | | |
| Pep-19 | MNQASMS | neutral | 0 | 5.02 | 974.18 | 0.222 |
| | (SEQ ID NO: 19) | | | | | |
| Pep-20 | PKGDENT | acidic | -1 | 3.99 | 966.08 | -1.344 |
| | (SEQ ID NO: 20) | | | | | |

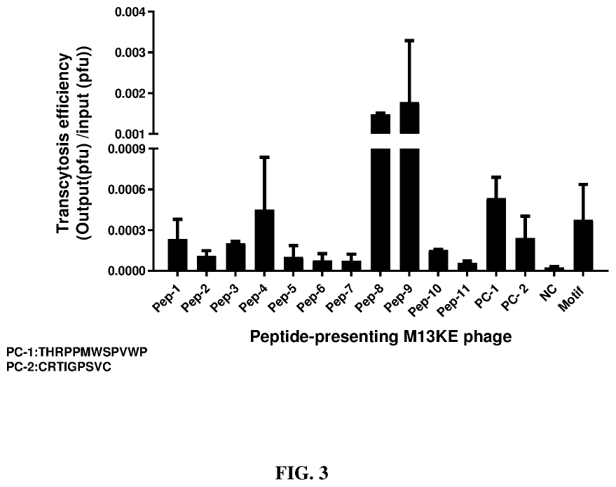

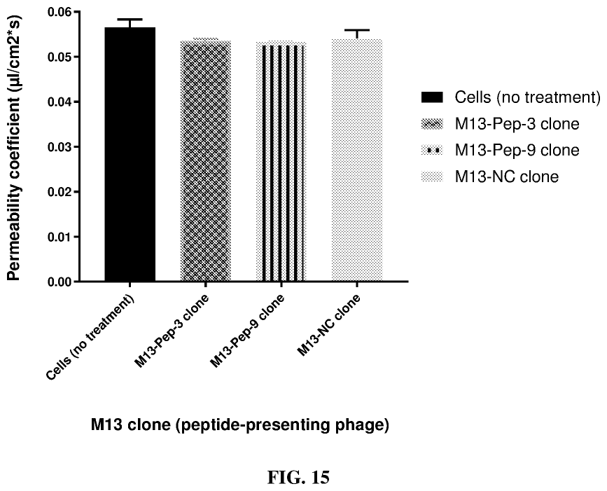

Transcytosis of select CX7C peptide-presenting phage in vitro. After identifying peptide sequences, the inventors confirmed the ability of individual peptide-presenting phage to shuttle across the BBB in vitro. The eleven most abundant and enriched sequences (denoted as Pep-1-Pep-11) and the consensus motif were individually cloned into the M13KE vector for peptide display. In addition, negative control NC, which demonstrated decreased frequency with successive rounds of panning (18 counts in round 1, not present in subsequent rounds), and two BBB shuttling peptides from other groups^{35,38}, were also cloned into M13KE. Transport of these individual peptide-presenting phage across hCMEC/D3 cells was quantified following the same transcytosis assay used for biopanning. The number of phage that shuttled across hCMEC/D3 over the total number of input phage for each M13 clone was calculated to compare their transcytosis or transport efficiency across the BBB (FIG. 3). In particular, Pep-and Pep-9 presenting phages demonstrated highest shuttling efficiency, with output to input ratios of 1.48×10⁻³ and 1.78×10⁻³, respectively. The five most frequent peptide-presenting clones (Pep-1 to Pep-5) had transcytosis efficiencies of 2.34×10⁻⁴, 1.09×10⁻⁴, 2.02×10⁻⁴, 4.51×10⁻⁴, and 1.01×10⁻⁴ respectively; the other clones amongst the top 11 had efficiencies ˜10−5. Here, the motif-presenting M13 clone had 3.76×10−4transcytosis ratio and the positive controls THRPPMWSPVWP (PC-1) (SEQ ID NO: 23) and CRTIGPSVC (PC-2) (SEQ ID NO: 24) had 5.34×10⁻⁴ and 2.42×10⁻⁴ respectively. As expected, the negative control NC demonstrated the lowest BBB shuttling efficiency, with a ratio ˜2.50×10⁻⁵ (FIG. 3). The consensus motif did not exhibit the highest level of transcytosis. The motif may be indicative of the composition and amplification bias of the library. The motif was present in all three rounds of biopanning, and the similar motif was in the naïve library and its amplification rounds. It has been demonstrated that M13 phage display libraries have limited diversity and have a bias for individual amino acids at specific positions. Similar to these findings, other libraries demonstrate less diversity at the first and last position⁵⁸ and compositional bias for specific amino acids^{59,60}. As observed by others⁶¹, the motif sequence-presenting M13 clone may not bind and shuttle across hCMEC/D3 better than the selected peptide-presenting phage, which may have a specific target or transport mechanism. To confirm that the BBB model remained intact during phage transcytosis, the inventors incubated hCMEC/D3 with tracer molecule dextran⁶² before and after phage incubation and measured its permeability (FIG. 15). Phage had negligible effect on the permeability of the BBB model.

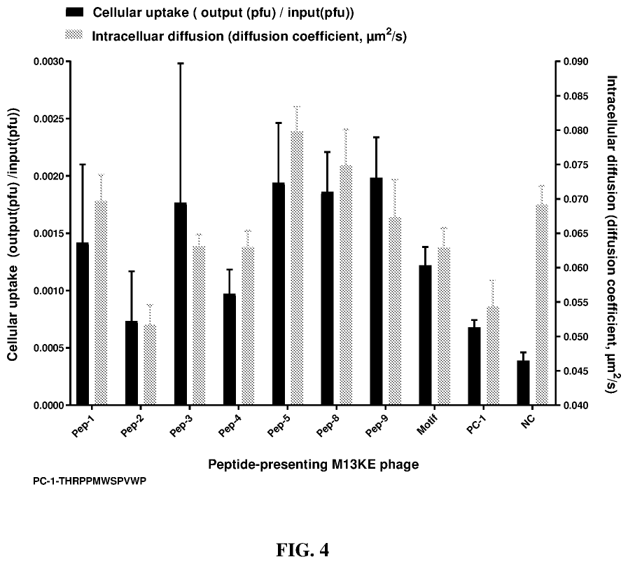

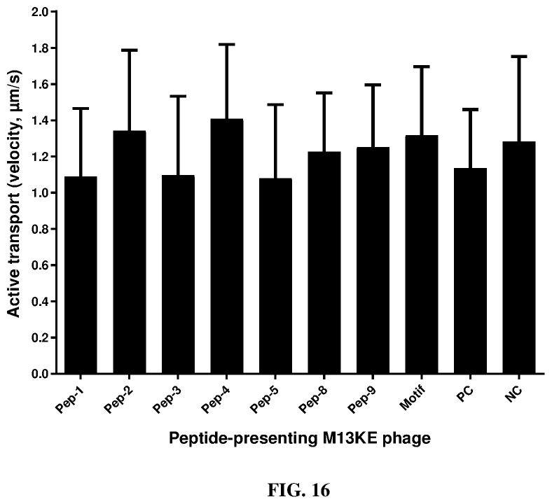

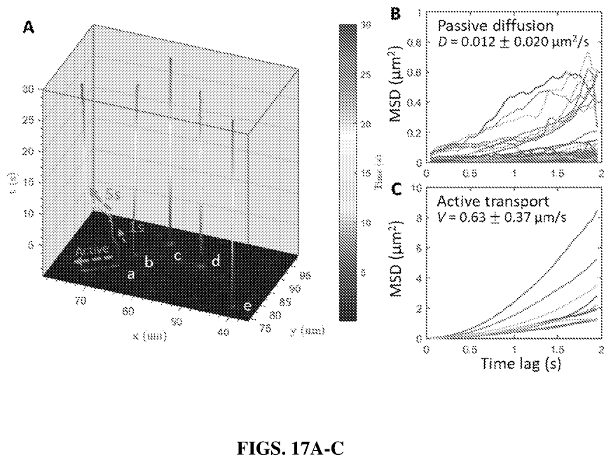

To study cellular uptake of transcytosed phage in hCMEC/D3 cells, the inventors tested phage that had transcytosis efficiencies above 10⁻⁴ (Pep-1-Pep-5, Pep-8, and Pep-9) and compared to control phage (NC and PC-1). Phage clones were incubated with confluent hCMEC/D3 cells, and internalized phage were collected and quantified relative to the amount of their input (FIG. 4). The selected clones demonstrated uptake ratios (i.e., the number of internalized phage/input phage) ranging from 5.66×10⁻⁴ to 2.07×10⁻³ (FIG. 4, black-filled bars). Interestingly, Pep-9 phage exhibited the highest cellular uptake with a ratio of 2.07×10⁻³, and the negative control NC phage exhibited the lowest uptake ratio of 4.17×10⁻⁴. Cellular uptake of the phage clones correlated with the efficiency of transcytosis (FIG. 3), which is expected since cellular uptake is part of transcytosis. In addition, the intracellular motion of phage clones was imaged and quantified using 2D particle tracking, which is able to track passive and active transport. Selected M13 clones were fluorescently labeled and incubated with confluent hCMEC/D3 cell monolayer for 1 h. Intracellular transport of these clones was recorded as 30 s movies, and the trajectories of intracellular motion for each clone was analyzed using a 2D particle tracking method^{63,64}. Here, the intracellular diffusion coefficient ranged from 5.17×10⁻²−7.98×10⁻² m²/s for the selected M13 clones (FIG. 4, pink-filled bars). The active transport behavior of the M13 clones can be extracted and calculated from particle tracking trajectories; selected phage clones had velocities ranging from 1.08-1.41 μm/s (FIG. 16). The velocities for active transport are within the observed values of active intracellular transport via motor proteins (0.5-2 μm/s) in EGFR trafficking⁶⁴ and other studies⁶⁵-67. The velocities for passive diffusion were within the range measured for confined diffusion, which was observed during events associated with ligand-receptor binding and active transport^{64,68}. The intracellular trajectories of a representative M13 clone and segmentation of their motion into active transport and passive diffusion were shown in FIGS. 17A-C. The intracellular trajectories of the phage clones and their calculated velocities suggested that identified M13 phage clones use active transport to shuttle across the BBB model.

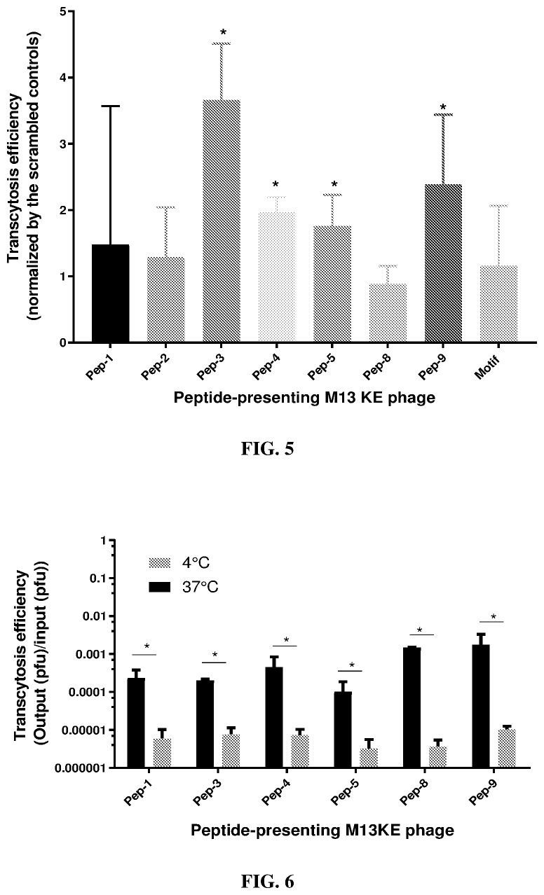

To confirm that transport across the BBB is sequence-specific, the transcytosis of phage clones (with transcytosis efficiencies above 10⁻⁴) was compared to their scrambled controls, i.e., phage-displayed peptide with same amino acid composition but in random order of the 7-mer (X7) in the CX7C region (FIG. 5). Here, Pep-3, Pep-4, Pep-5, and Pep-9 clones exhibited statistically greater transcytosis efficiency ((3.99±0.44)×10⁻⁴, (5.87±0.66)×10⁻⁵, (5.00±0.12)×10⁻⁵, (7.71±0.23)×10⁻⁵, respectively) than their respective scrambled controls ((1.13±0.26)×10⁻⁴, (3.03±0.64)×10⁻⁵, (3.00±0.91)×10⁻⁵, (3.61±1.58)×10⁻⁵; p≤0.05). If phage clones did not demonstrate specific transport in hCMEC/D3 cells, altering the sequence order would not change their transport⁶⁹⁻⁷¹, as seen with other phage-displayed peptides ligand-target binding studies^{72,73}. This result suggests that these highlighted clones have specific interactions with targets present on the hCMEC/D3 cells.

To determine if selected phages transport across hCMEC/D3 cells through a temperature-dependent mechanism, the transcytosis assay was performed at 37° C. and 4° C. Transcytosis efficiency was 25 to 402-fold higher for the phage clones at 37° C. than 4° C. (FIG. 6). Of note, Pep-8 and Pep-9 phage clones demonstrated the greatest difference in their transport, with a 402- and 169-fold decrease at 4° C., respectively. These findings are in agreement with other peptide-mediated transport studies with D1 peptide⁷⁴ and tympanic membrane transport peptide TMT-3⁷⁵, which showed energy-dependent active transport. Combined with the results in FIG. 4 and FIG. 16, the inventors' data suggest that phage clones were actively transported across hCMEC/D3 cells.

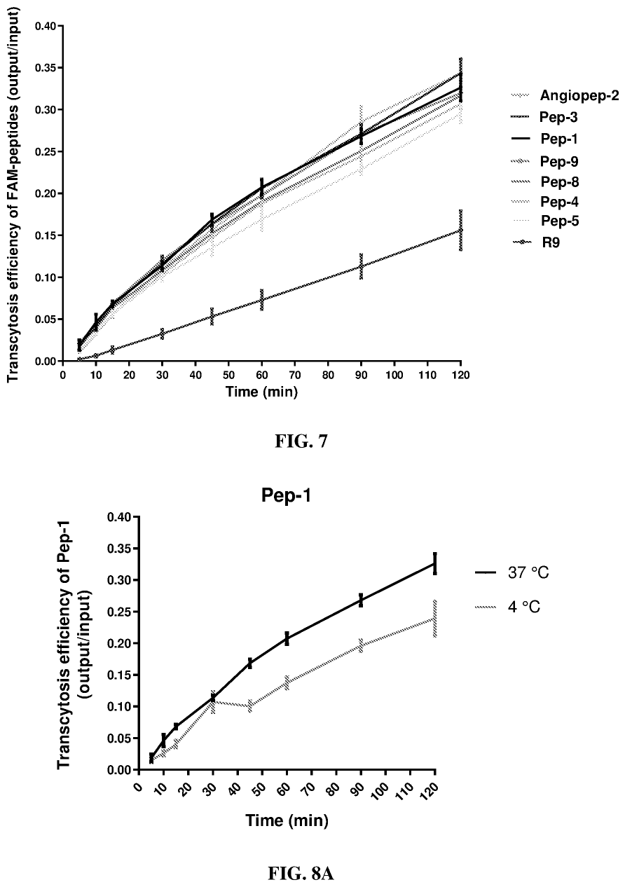

Transcytosis of selected peptides against BBB in vitro. While the inventors' prior experiments focused on validating transport of peptide-presenting phage in vitro, they wanted to confirm the ability of the peptides to facilitate BBB transport without the structural context of the M13 phage. Here, fluorescently-labeled free peptides, Pep-1, Pep-3, Pep-4, Pep-5, Pep-8, Pep-9, cell-penetrating peptide nona-arginine (R9)⁴⁵, and Angiopep-2⁷⁶ were synthesized and tested for their ability to traverse the hCMEC/D3 cells. R9 is an arginine-rich peptide that efficiently binds to cells via electrostatic interactions and has been shown to penetrate the cellular membrane by macropinocytosis⁷⁵ at low nanomolar to micromolar concentrations and by pore-forming translocation into the cytosol at higher concentrations^{77,78}. Angiopep-2, which has been shown to cross the BBB, is a rationally designed peptide that was identified from sequence alignment with human kunitz domain and aprotinin, an inhibitor to low-density lipoprotein receptor^{76,79}. Angiopep-2 has been tested from in-vivo studies and in clinical trials to shuttle different drugs such as small molecule paclitaxel⁸⁰, neurotensin peptide⁸¹, and anti-HER2 antibody⁸², across the BBB. Using the transcytosis assay described earlier, the equivalent molar weight of each FAM-labeled peptide was incubated with confluent hCMEC/D3 cells in the donor compartment of the transwell system and their fluorescence was measured in the receiving compartment at a series of time points up to 120 min. The ratio of output to input fluorescence intensity was calculated to represent the ability of each peptide to shuttle across the in vitro BBB and account for any potential differences between fluorescent labeling efficiency of synthesized peptides. The output/input ratio for each peptide at different time points varied from the range of 0.00159-0.361. At 120 min, the output/input for all the tested peptides was within the range of 0.130-0.361. Interestingly, the selected peptides demonstrated transcytosis efficiency comparable to Angiopep-2 up to 60 min. Pep-3 and Pep-9 had equivalent transport compared to Angiopep-2 at 90 min, whereas only Pep-3 had similar transport at 120 min (FIG. 7, p<0.05, two-way ANOVA). All CX7C peptides demonstrated improved transport compared to R9 throughout the duration of the assay (except at 5 minutes; FIG. 7, p<0.05, two-way ANOVA). However, while R9 has been used as a cell-penetrating peptide^{21,83}, there are no reports of the ability of R9 to exocytose and exit the BBB. These results suggest that the CX7C peptides are more efficient to shuttle across hCMEC/D3 cells than R9, and certain CX7C peptides exhibited similar transport to Angiopep-2.

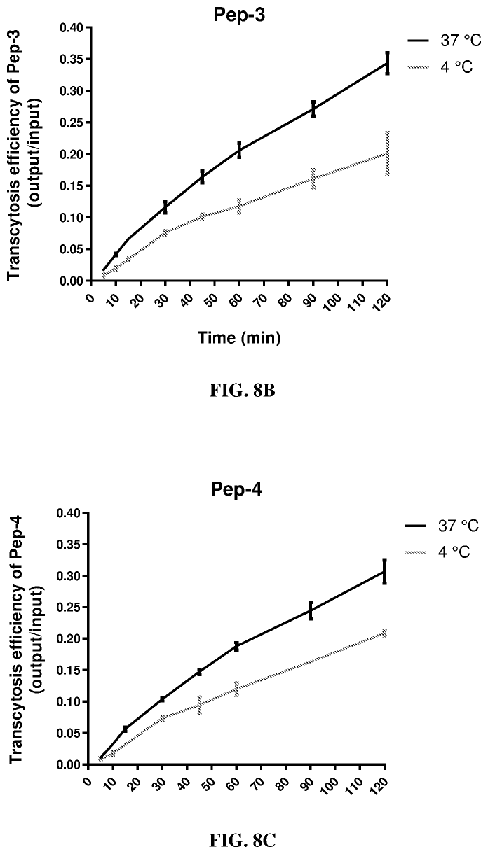

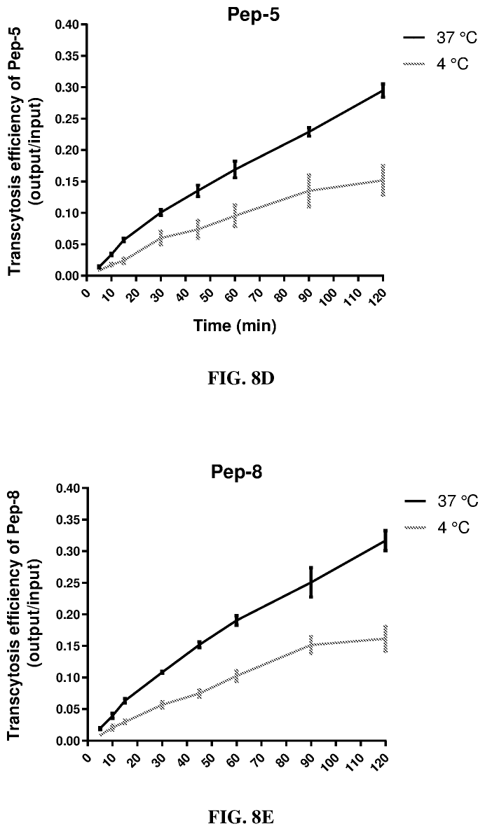

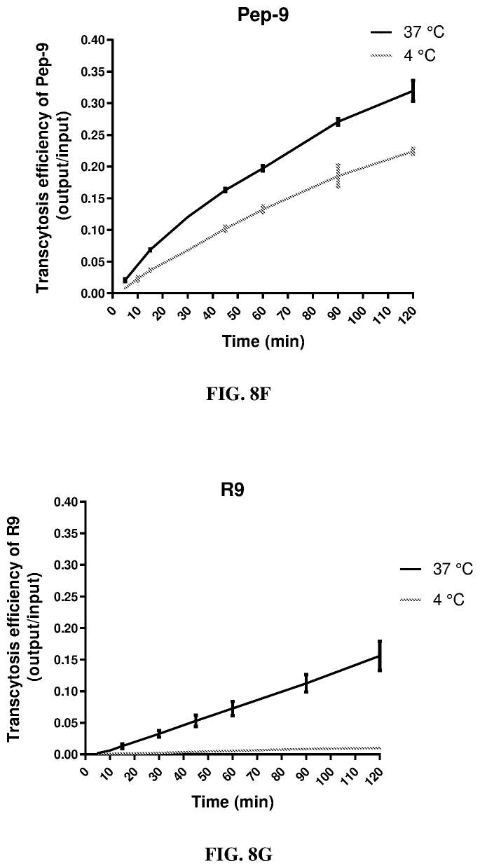

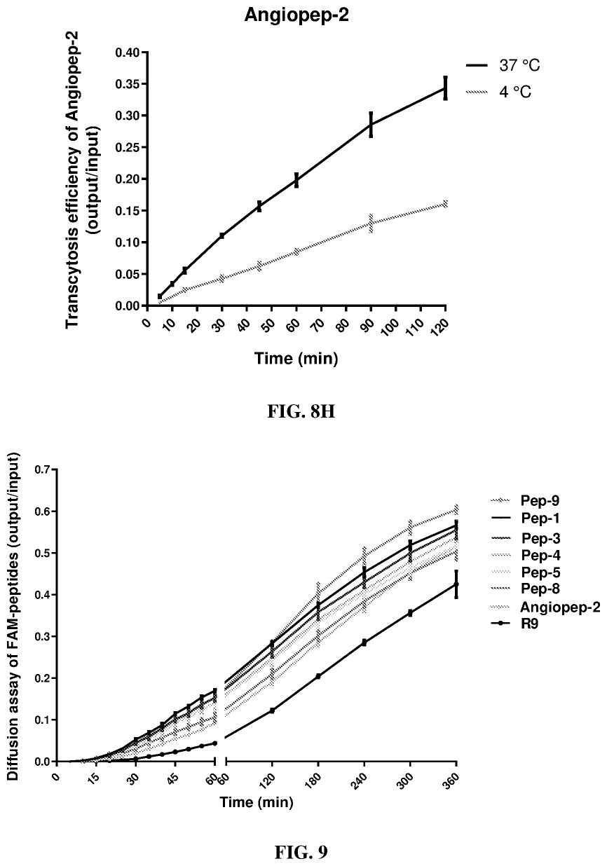

To confirm that selected peptides transport across the BBB through energy-dependent pathways, selected FAM-CX7C peptides were incubated against hCMEC/D3 cells at 37° C. and 4° C. respectively, and transcytosis efficiency was quantified as before. All peptides demonstrated decreased transport at 4° C. (FIGS. 8A-H). R9 demonstrated the greatest decrease in transport at 4° C., with an almost 9-fold reduction in transcytosis efficiency; this is most likely due to its inability to be internalized by the cells. The other peptides exhibited ˜0.5- to 1.4-fold reduction in transcytosis efficiency at 4° C.

These tested peptides, like cell penetrating peptides (CPPs), may involve two different pathways for internalization: (1) translocation at 4° C. or (2) endocytosis and translocation at 37° C.⁸⁴. It is known that R9 endocytosis is temperature dependent and requires cell-surface glycosaminoglycans (GAGs). The inventors' study confirmed that R9 undergoes temperature-dependent transport against in vitro BBB model. In the initial step of cellular uptake, there can be fluid-phase and receptor-mediated uptake⁸⁵; receptor-mediated uptake is (1) saturable with respect to extracellular concentrations of the ligand, (2) non-linear with time, and (3) is temperature-independent⁸⁴. Angiopep-2 was previously identified to transport across the BBB using low-density lipoprotein (LDL) receptor-mediated transcytosis⁷⁶. Here, free Angiopep-2 demonstrated temperature-independent transport against hCMEC/D3 cells (FIGS. 8A-H), with only 1.4-fold decrease of transcytosis efficiency at 4° C. The discovered peptides demonstrated comparable temperature-independent behavior, with only ˜0.5- to 1.0-fold reduction in the BBB transport at low temperature. It indicates that receptors on the hCMEC/D3 may involve in the cellular uptake of these CX7C peptides. Since transcytosis includes cellular uptake, intracellular trafficking, and exocytosis, future studies will need to elucidate the latter two processes of the peptide transcytosis. In addition, identification of the binding target of the peptides will be critical to understanding its role in BBB transport.

Diffusive transport of selected peptides through the extracellular matrix. After penetrating the BBB, molecules must also traverse the extracellular matrix (ECM) in the extracellular space of the brain to reach the parenchyma. Since transport through brain ECM is mainly driven by diffusion^{3,86}, the inventors performed two experiments to study the diffusion of the selected peptide through Matrigel, an ECM mimic that has been extensively used to study diffusive transport of solutes and nanoparticles⁸⁷⁻⁸⁹. In one experiment, the equivalent molar weight of each FAM-labeled peptide was added to the donor compartment of a transwell insert coated with Matrigel and allowed to diffuse into the receiver compartment of the transwell. The fluorescent intensity of peptides was measured in the receiver compartment up to 6 h. The ratio of output to input fluorescence represents the efficiency of each peptide to diffuse through Matrigel. For the duration of the study, R9 diffused the slowest through Matrigel among all tested peptides (FIG. 9, two-way ANOVA, p<0.05). Importantly, Pep-1, Pep-3, Pep-4, Pep-5, Pep-9 peptides exhibited improved transport through Matrigel as compared to Angiopep-2 (except at 300 and 360 min timepoints, where Pep-5 was comparable to Angiopep-2); Pep-8 demonstrated equivalent transport to Angiopep-2 during the entire time course (two-way ANOVA, p<0.05).

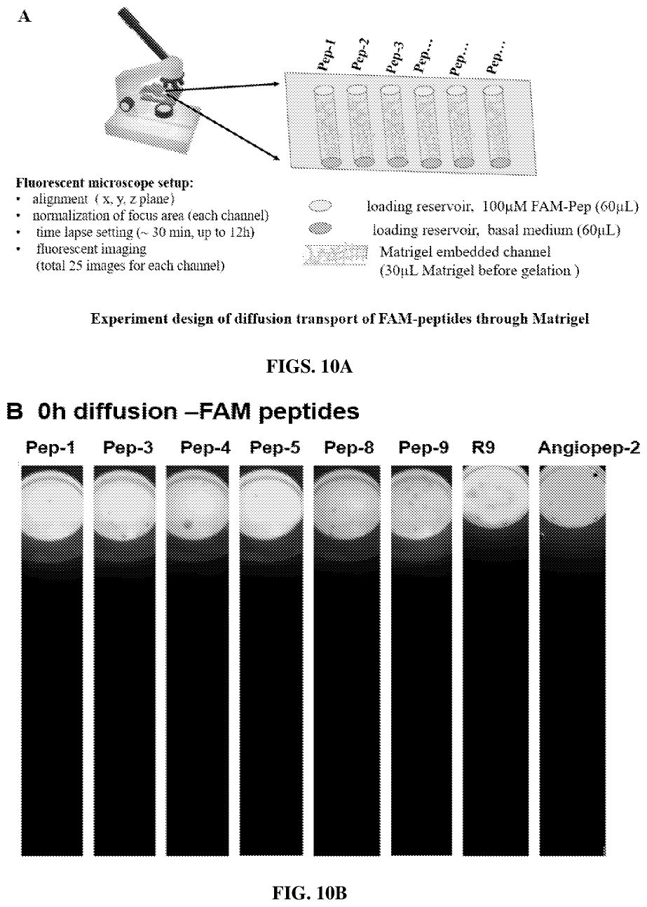

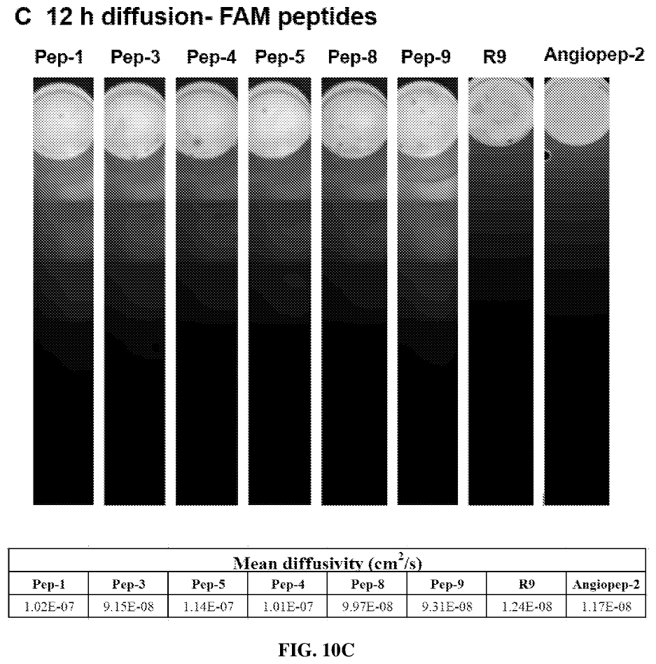

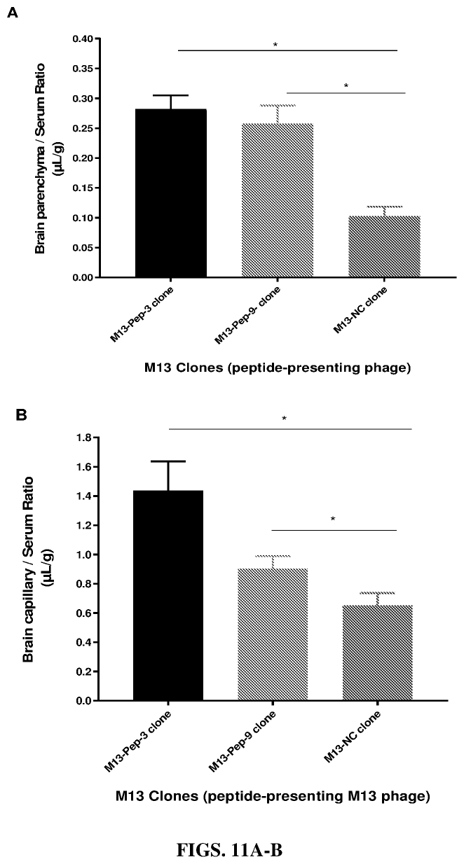

Next, diffusive transport of the peptides was imaged in a microfluidic chamber slide with embedded Matrigel to exclude possible interactions between the peptides and the polycarbonate membrane insert from the transwell. Here, the equivalent molar weight of FAM-CX7C peptides and controls were incubated with Matrigel embedded in the channels of the chamber slide under sink conditions⁹⁰ (FIG. 10A). Whole field-of-view fluorescent images of each channel were taken every 30 min up to 12 h in a time-lapse mode for a total of 25 fluorescent images for each channel. Representative images at 0 and 12 h are shown in FIGS. 10B-C for each peptide. From the collected images from time-lapse imaging, the inventors generated movies to demonstrate transport of FAM-labeled peptides through the Matrigel embedded channel for the entire 12 h. All selected CX7C peptides migrated faster than R9 and Angiopep-2 during the 12 h diffusion assay. From the time-lapse imaging, the mean diffusivities of the peptides were calculated with a custom Matlab script (Table A) based on the solution to Fick's Second Law⁹¹, presented as equation (1)⁹² shown below.

$$C ( x , t ) αerfc ( \frac{x}{2 \sqrt{{D}_{eff} t}} ) \qquad ( 1 )$$

where C is the fluorescence intensity of the FAM-labeled peptides, x is the penetration distance at a given time t, and D_{eff} is the effective diffusivity. The diffusivity of each selected peptide was approximately 10⁻⁷ cm²/s, which was ˜10-fold higher than R9 (1.24×10⁻⁸ cm²/s) and Angiopep-2 (1.17×10⁻⁸ cm²/s) (FIGS. 10A-C). Selected CX7C peptides demonstrated greater diffusivity than controls, most likely due to their weaker intermolecular interactions with the ECM. Matrigel has a net negative charge, and the selected CX7C peptides do not possess highly positive charged residues (net charge of 0 to +2, Table 1). The highly positive net-charge of R9 (+9) facilitates efficient cell binding to capillary endothelium²¹, but charge effects can adversely impact exocytosis and diffusion through the brain parenchyma. Also, although Angiopep-2 can enter the brain parenchyma, engineered Angiopep-related peptides with +4 and +6 net-charge demonstrated significantly less distribution into the brain parenchyma due to their accumulation with the negatively-charged blood vessels⁷⁹. Another study reported that lactoferrin protein exhibited hindered diffusivity through the extracellular space due to charge interactions. Lactoferrin has basic amino acids near the N-terminus that bind to negatively charged heparin and heparin sulfate present in the ECM, thereby inhibiting its diffusion through the extracellular space of the brain^{3,22}. While charge may impact diffusion, the length, confirmation, and amino acid composition of the peptides may alter diffusion transport. The discovered peptides (m.w. ˜1 kDa) have cysteine-constrained cyclic structures, while Angiopep-2, TFFYGGSRGKRNNFKTEEY (m.w. ˜2.3 kDa) (SEQ ID NO: 26) has a linear alpha-helix structure. Compared to the linear peptide, cyclic peptides, absent of exposed free N- and C-termini, have decreased potential interactions between the peptides and solvent; cyclic peptides have more compact confirmation, which allows them to diffuse faster due to the reduced collision profile in the solution⁹³. In addition, since Angiopep-2 has a greater number of amino acids, it can have more multivalent interactions and bind to the ECM and due to its larger size, have slower diffusivity through Matrigel, as calculated from Stokes-Einstein equation. These collective findings substantiate that charge-based interactions, size, and/or dimension, can impact diffusion through the ECM, which is critical for drug delivery through the extracellular space¹⁷.

From multiple diffusion assays (FIG. 9 and FIGS. 10A-C), the inventors observed that their peptides diffused better than Angiopep-2 and R9. Any discrepancy in diffusive transport between the two assays is likely due to the presence of a negatively charged polycarbonate membrane insert in the transwell plate, which may impact binding of peptides, such as R9 (+9 net charge) and transport through the barrier.

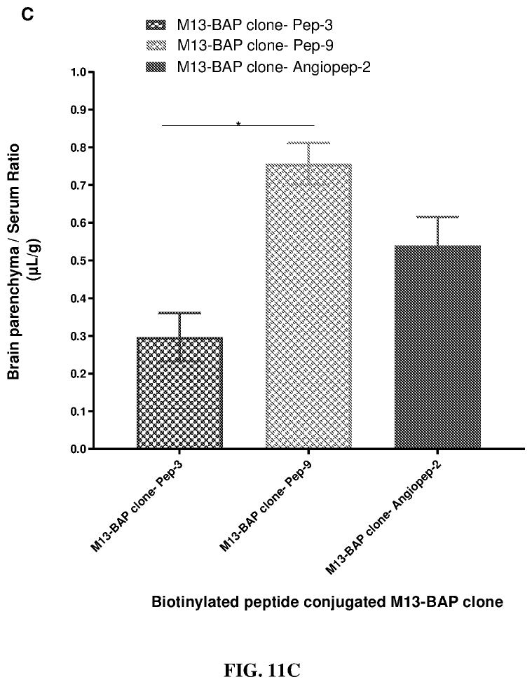

Select peptide-presenting M13 phage penetrate the BBB and enter the brain parenchyma in vivo. From the prior studies, Pep-3 and Pep-9 presenting phage were the most promising clones for BBB penetration and ECM diffusion in vitro, and they were subsequently tested for their ability to penetrate the BBB and reach the brain parenchyma in vivo. Pep-3, Pep-9 and negative control M13 phage clones were injected into healthy Balb/C mice. Thirty minutes post injection, mice were sacrificed, perfused, and their brains were harvested for capillary depletion to separate the brain parenchyma from the capillaries. The amount of phage in the parenchyma, capillary, and blood serum was quantified by standard phage titering. Pep-3 and Pep-9 clones had significantly higher accumulation in the brain parenchyma than the negative control, with brain parenchyma/serum ratios of 0.28, 0.26, and 0.10 μl/g, respectively (FIG. 11A). The brain capillary/serum ratio was 1.44 and 0.90 μl/g for Pep-3 and Pep-9 peptide-presenting M13 phage respectively, which were both significantly higher than the negative control (0.65 μl/g) (FIG. 11B). The phage clones exhibited similar distribution in the blood 30 minutes after injection, which suggests they have similar circulation half-life. As a result, the difference of the brain uptake suggests that Pep-3 and Pep-9 presenting phage mediate BBB transport into the brain parenchyma.

The amount calculated in the brain parenchyma/serum ratio and brain capillary/serum ratio for Pep-3 and Pep-9 phage clones were on the same order of magnitude to reported values from other BBB transport studies. BBB permeable peptide-nesfatin demonstrated a parenchyma/serum ratio ˜1.05 μl/g and capillary/serum ratio ˜0.51 μl/g⁹⁴. In other studies, epinephrine-mediated delivery of beta-glucuronidase had ˜1.04 and 1.08 μl/g⁹⁵ in the parenchyma and capillary, respectively, and sulfamidase enzyme had the values ˜0.87 and 0.17 μl/g⁹⁶, respectively. Epinephrine and sulfamidase both shuttle across the BBB through mannose 6-phosphate/insulin-like growth factor 2 receptor-mediated transport⁹⁴⁻⁹⁶.

Next, the inventors measured BBB shuttling of phage displaying Pep-3 and Pep-9 compared to Angiopep-2. Since Angiopep-2 was identified from rational design⁷⁶ and cannot be genetically engineered for display on the pIII coat protein of M13 phage, it was necessary to develop an alternative strategy for peptide display to compare BBB transport. Here, the inventors cloned a 15-amino acid biotin acceptor peptide sequence (BAP; sequence: GLNDIFEAQKIEWHE)⁹⁷ (SEQ ID NO: 25) on pIII of M13 phage that can be enzymatically biotinylated with BirA biotin-protein ligase⁹⁸. Subsequently, the inventors were able to site-specifically and covalently conjugate biotinylated Pep-3, Pep-9, and Angiopep-2 peptides onto the BAP sequence displayed on M13 phage and directly compare their uptake in the brain.

After intravenous injection, they isolated brain parenchyma by capillary depletion and quantified the brain uptake. The brain parenchyma/serum ratios were 0.30, 0.76, 0.54 μL/g (FIG. 11C), while the brain capillary/serum ratios were 0.87, 1.13 and 1.09 μL/g (FIG. 11D) for M13 phage conjugated with biotinylated Pep-3, Pep-9, and Angiopep-2 peptides, respectively. Pep-9 conjugated phage demonstrated significantly higher accumulation in the brain parenchyma than phage conjugated with Angiopep-2 (FIG. 11C). The variation of brain accumulation between the peptide-presenting phage and the biotinylated peptide-conjugated phage may be due to the steric hindrance and/or altered binding accessibility of the peptides to the targets on the BBB of the biotinylated peptide-conjugated phage^{99,100}.

The inventors' select M13 clones traverse the BBB and enter the brain parenchyma compared to controls, but the kinetics of uptake require further optimization. M13 phage demonstrates a short systemic circulation half-life in vivo, and by increasing its half-life, accumulation into the parenchyma is expected to increase. While the amount of M13 phage penetrating into the brain parenchyma can be improved, it is important to note that the Pep-3 and Pep-9 peptides can ferry this large macromolecule (molecular weight of M13 phage ˜16.4 MDa, with dimensions of ˜900 nm length, 6-7 nm diameter) across the BBB.

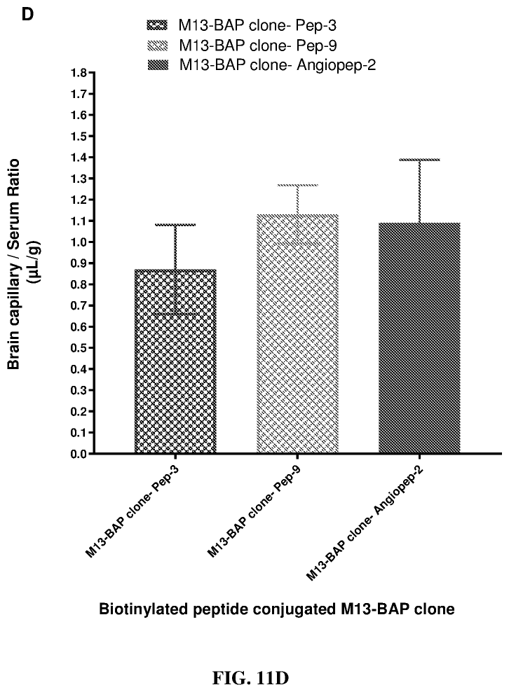

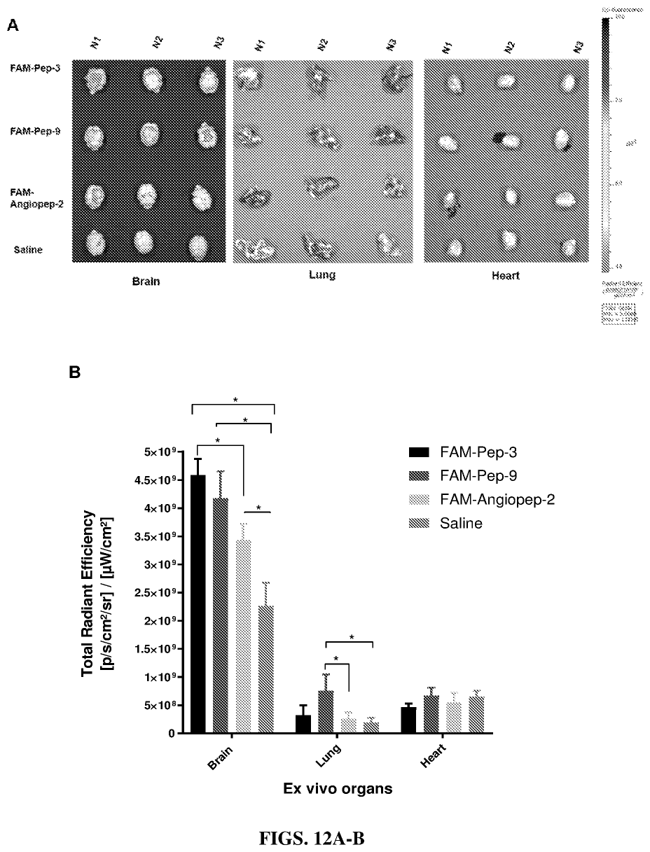

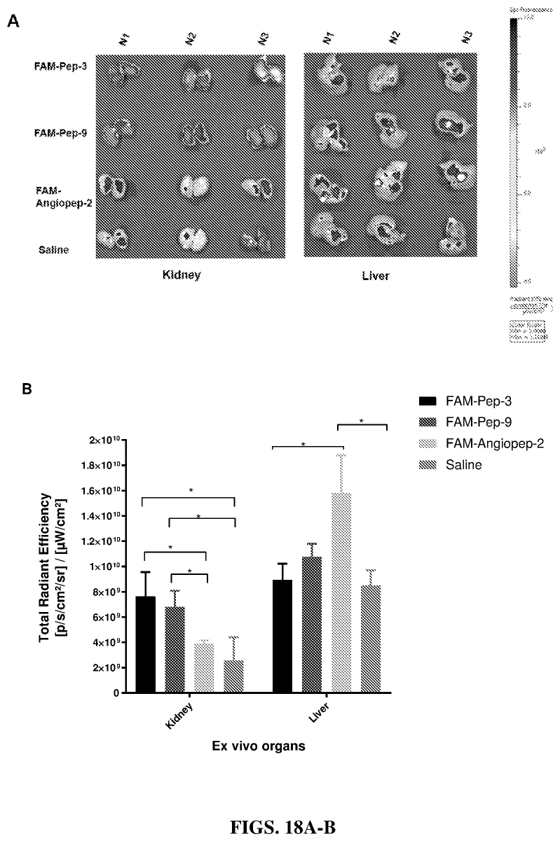

Biodistribution of free FAM-labeled Pep-3, Pep-9 and Angiopep-2 peptides in vivo. In addition to quantifying phage uptake into the brain parenchyma, the inventors wanted to measure in vivo biodistribution of free peptides. Equivalent molar weight of fluorescently labeled Pep-3, Pep-9 and Angiopep-2 peptides were dosed intravenously and circulated for 30 min¹⁰¹⁻¹⁰³. Tissues were harvested and fluorescent images of the brain, lung, heart, kidney and liver of the mice in peptide dosing and control groups were acquired (FIG. 12A and FIG. 18A). Manual regions of interest (ROI) were drawn over the entire organ from each mouse. Total radiant efficiency of each ROI was calculated and plotted in FIG. 12B. Pep-3, Pep-9 and Angiopep-2 demonstrated effective brain distribution (peptide groups vs saline group, P<0.05). Particularly, Pep-3 had significantly higher brain accumulation than Angiopep-2 (P<0.05); Pep-9 demonstrated higher, but not statistically significant, accumulation than Angiopep-2. Pep-3 and Pep-9 demonstrated higher accumulation in the brain with comparatively low to negligible accumulation in other highly vascularized lung and heart tissues (FIGS. 12A-B). Pep-3 had no accumulation in the heart and lungs, whereas Pep-9 exhibited minimal uptake in the lungs. As expected, the peptides are eliminated by renal and hepatic clearance, as evidenced by the fluorescence signal of peptides in the kidney and liver (FIGS. 18A-B). The kinetics of peptide-target receptor engagement may explain why Pep-3 and Pep-9 demonstrated improved accumulation in the brain compared to Angiopep-2, and there may be differences of free peptide binding compared to peptide-presenting phage binding (FIGS. 11A-D) due to the structural context of the phage and the multivalent display of peptide on phage. However, in the future, more comprehensive pharmacokinetics of the peptides will be measured to understand their circulation, distribution, and elimination.

Multiple in vitro and in vivo studies have been performed to validate the ability of identified peptides, in particular, Pep-3 and Pep-9, to cross the BBB and penetrate into the brain parenchyma. The inventors observed that Pep-9 phage has about 10-fold higher transcytosis efficiency than Pep-3 clone in vitro, but Pep-3-presenting phage exhibited higher uptake than Pep-9 in the brain parenchyma in vivo. Since biopanning in vitro was done without a priori knowledge of the target receptor, the binding target of Pep-3 and Pep-9 are unknown; however, it is known that the human-derived hCMEC/D3 cells have different expression levels of transporters and receptors compared to rodent BBB models^{41,51}. The difference in target expression between species could contribute to the difference in transport in vitro and in vivo, and future studies would be needed to test this hypothesis. The differences in brain accumulation between biotinylated peptides conjugated on M13 (FIGS. 11A-D) and free peptides (FIGS. 12A-B) suggest that when peptides are modified, they may exhibit altered transport across the BBB. Here, the free peptides are chemically modified with FAM on the N-termini, and the biotinylated peptides are enzymatically conjugated to the biotin acceptor peptide on M13 phage. Any type of modification to the BBB-shuttling peptides may influence the binding or recognition of the peptides to the targets on the brain capillary endothelium, and work involving therapeutic carriers such as nanoparticles will have to take this into consideration when measuring BBB transport^{100,104,105}.

### Example 3—Conclusions

Treatment for neurological illnesses remains poor due in part of the inability of therapeutics to distribute throughout the compartments of the brain to reach the diseased site. As a result, drug delivery remains a major challenge to the successful treatment of CNS diseases. The majority of therapeutics are unable to traverse either the BBB or blood-cerebrospinal fluid barrier and effectively diffuse through the surrounding extracellular space to reach the brain parenchyma. Current brain delivery strategies about BBB transport mainly focus on transiently opening the BBB using focused ultrasound¹⁰⁶ and hyperosmotic agents¹⁰⁷, on bypassing the barriers through local delivery¹⁰⁸, or receptor-mediated transport. These studies address either transport across the BBB or circumventing the BBB to study diffusive transport through the ECS. However, there has been no strategy that explicitly that addresses delivery of molecules through both barriers of the BBB and the extracellular space into the brain parenchyma, which is critical to achieving therapeutic concentrations throughout the brain.

Here, for the first time, the inventors used a combinatorial approach to identify peptides that transport across the BBB and diffuse through ECM in vitro and in vivo. They address this challenge by using phage display with next-generation sequencing to identify peptide-presenting phage that transport across the BBB and diffuse through the ECM. Through in vitro biopanning, peptides were identified that functionally penetrate the BBB and ECM. While prior strategies have used phage display in vitro³⁴ and in vivo¹⁰⁹ biopanning to screen BBB-targeting peptides that were mainly identified by Sanger sequencing, they did not effectively account for the necessity for peptides to transport through the extracellular space of the brain microenvironment after crossing the BBB. Here, the inventors demonstrated that their selected CX7C peptides, in particular, Pep-3 and Pep-9, exhibited greater transport across the BBB than the cell-penetrating peptide R9 and comparable with Angiopep-2, which has been clinically tested to improve drug delivery for brain-associated cancers^{80,81,82}. Importantly, the identified CX7C peptides showed improved diffusivity through the extracellular matrix than R9 and Angiopep-2. Angiopep-2 has been shown to reach the brain parenchyma⁷⁶ but demonstrates slower diffusion through ECM than the inventors' peptides. It is feasible that interactions between Angiopep-2 and ECM hinder diffusivity compared to the inventors' smaller nine-amino acid cyclic peptides. In in vivo studies, Pep-3 and Pep-9 presenting M13 phage clones intravenously administered in healthy mice were able to cross the BBB, extravasate, and enter the brain parenchyma. Pep-3 and Pep-9 presenting phage exhibited brain parenchyma/blood serum ratios comparable to the other BBB permeable macromolecules that underwent receptor-mediated BBB transport. In particular, Pep-9 conjugated phage had higher accumulation in the brain parenchyma than Angiopep-2 conjugated phage, which highlights the attractiveness of using the inventors' peptides for improved BBB transport of macromolecules. In addition, a higher concentration of free Pep-3 and Pep-9 peptides were able to accumulate in the brain compared to Angiopep-2 peptide. While further work is needed to elucidate the specific target engagement and pathway of BBB transport and ECS transport in vivo of these peptides, these collective findings suggest that Pep-3 and Pep-9 may specifically bind to the BBB and mediate transport into the brain parenchyma. Since these peptides facilitate transport of the “large” M13 phage across the BBB and into the parenchyma, these brain-penetrating peptides are an attractive and broad platform that can potentially transport and deliver previously BBB impermeable drugs, including macromolecules such as enzymes, antibodies, and nanoparticles, across the BBB and through extracellular space to treat diseases of the CNS.

### Example 4—Materials and Methods

Materials. Anti-mouse CTLA-4 antibody (CD152, clone 9D9, cat. #BP0164) was purchased from BioXcell. Click-easy MFCO-N-hydroxysuccinimide ester (NHS) (cat. #LK4300, m.w. 380.41 g/mol) was produced from Berry & Associates, Inc. Sephadex G-25 in PD-10 Desalting Columns was bought from GE (cat. #17085101); Azide modified Pep-9 (CLWRPAADC-LysN3 (SEQ ID NO: 27), m.w. 1185.40 g/mol) was synthesized with Fmoc chemistry and lyophilized to have 97.2% purity by Lifetein LLC. Float-A-Lyzer G2 Dialysis (Part no. G235034) was purchased from Spectrum Laboratories, Inc. Amicon Ultra-4 centrifugal filters (UFC805024) was produced from Millipore Sigma. Precast polyacrylamide gel 10% (cat. #456-1036), 7.5% (cat. #456-1026), SDS-PAGE running buffer (cat. #1610732), 4×loading buffer (cat. #161-0747) and protein ladder (cat. #161-0377) were purchased from Bio-Rad Laboratories. 10×Bolt sample reducing agent (500 mM Dithiothreitol (DTT), Ref #B0009), TMB-ELISA (Ref #34028) and Maxisorp ELISA plate (cat. #44-2404-21) were bought from Thermo Fisher Scientific. Mouse CTLA-4 protein (cat. #50503-M08H) was produced from Sino Biological Inc. Rabbit anti-mouse IgG (HRP)-ab6728 was purchased from Abcam. cOmplete™ ULTRA tablets complete Protease Inhibitor Cocktail was produced from Roche. Dextran (MW=200 k˜300 k) was purchased from MP Biomedicals (cat. #101514). BD microtainer with serum separation additive was produced from BD (cat. #365967).

IgG-Pep-9 conjugate formulation development. Anti-mouse CTLA-4 antibody, as the IgG model molecule was employed to develop IgG-Pep-9 conjugate formulation by copper-free click chemistry (Regina et al., 2015b) with modifications. Step1, for IgG-Pep-9 group, 1 ml of 3 mg/ml Anti-CTLA-4 antibody (IgG) (diluted in InVivoPure dilution buffer pH=7.0, BioXcell) was modified with 3.65 μL MFCO-NHS (stock 25 mg/ml prepared in DMSO) (12 equivalent), incubate for 6 h with 2 rpm orbital shaking at room temperature (RT); For IgG group 3.65 μL DMSO was added to 1 ml of 3 mg/ml IgG and continued with the same procedures as IgG-Pep-9 group. Desalting columns were conditioned with Milli-Q water to purity the MFCO-NHS modified IgG and free IgG molecules. Amicon 50K MWCO filter was used to concentrate the desalted IgG-Pep9 and IgG formulations and buffer exchanged with InvivoPure dilution buffer (pH=7.0). Step2, for IgG-Pep-9 group, 7.81 μL of Pep-9-Lys-N3 (24.3 mg/ml stock in DMSO, 8 equivalent) was added to the MFCO-NHS modified IgG formulation and incubated for 12 h with 10 rpm orbital shaking at RT. For IgG only group, 7.81 μVL DMSO was added to the IgG group from step 1 and went through the same procedures as IgG-Pep-9 group. Dialysis (50K MWCO) was run to purify the IgG-Pep-9 and free IgG for 24 h at 4° C.

Non-reducing and reducing SDS-PAGE. Mini protein tetra cell system (Bio-Rad) was used to run the sodium dodecyl sulfate-polyacrylamide gel electrophoresis (SDS-PAGE). In non-reducing SDS-PAGE, IgG-Pep-9 and IgG formulations were loaded at 3 μg/lane to 7.5% gel; meanwhile, 10 μL protein ladder/lane was also loaded to run at 120 V for 40 min ˜1 h. For reducing SDS-PAGE, before running the gel electrophoresis, 3 μg of IgG-Pep-9 or free IgG formulation were reduced by incubation with 50 mM DTT at 70° C. for 10 min. 10% gel was used to run the IgG samples for 30 min at 100 V.

Liquid Chromatography-Mass Spectrometry (LC-MS). 6˜8 μg/injection of intact IgG-Pep-9 or free IgG, reduced IgG-Pep-9 or free IgG were run at Ultima 3000 (Dionex) liquid chromatography coupled with Orbitrap fusion Mass spectrometry system (Thermo Fisher Scientific). Mobile phases were 0.1% formic acid (FA) in water (A) and 0.1% FA in acetonitrile (B), with B gradient ranged 5-95% within 15 min, reverse phase column was protein Microtrap (Michrom BioResources Inc.). Built-in ESI ion source was used to ionize the non-reduced or reduced IgG/IgG-Pep-9 samples. Ion trap (IC) and orbit trap (FT) mass spectrum were acquired from the LC-MS system. Protein spectrum deconvolution was analyzed to represent the abundance of each mass in the sample.

Dynamic light scattering (DLS). Purified IgG and IgG-Pep-9 formulations at a concentration around 100˜500 μg/ml were measured by Malvern Zetasizer Nano with size measurement. 100 μL of each formulation was loaded into a disposable ZEN0040 cuvette. 173° C. backscatter and 11 runs were set to measure the size distribution by intensity for each sample at 20° C.

In vivo study. All the procedures in the animal study were approved by the Institutional Animal Care and Use Committee (IACUC), the University of Texas at Austin. Female Balb/C mice (6˜8 weeks) were injected intravenously with IgG-Pep-9 or free IgG formulation at dose 10 mg/kg body weight by tail vein injection and allowed to circulate for 24 h. At 24 h time point, each mouse was euthanized with 1.5˜5% CO₂, 300˜500 μL blood was collected by heart puncture. Transcardial perfusion was run at 2.5 ml/min infusion rate by a syringe pump with a physiological buffer (Triguero et al., 1990). The whole brain was harvested and weighted before storing in 2 mL physiological buffer with protease inhibitor prepared in a 20 mL scintillation vial before capillary depletion. Tissue homogenizer (Fisher scientific, FSH125) was used to homogenize the whole brain for 5˜8 strokes. 2 mL of 26% dextran (prepared in the physiological buffer) was added to the tissue homogenate and homogenized for additional 3˜5 strokes. All the tissue homogenization procedures were performed on ice. The final brain homogenate was transferred to a 15 mL centrifuge tube and centrifuged at 5400 g for 15 min at 4° C. Brain parenchyma (supernatant) and capillary (pellet) were separated by the density-driven centrifugation. The supernatant was transferred to a new 15 mL centrifuge tube after centrifugation. All the tissue samples were kept at −80° C. before futher analysis.

ELISA. Maxisorp ELISA plate was coated with 100 ng/well (100 μL of 1 ng/μL, diluted in 0.1 M carbonate buffer, pH=9.0) mouse CTLA-4 protein for overnight at 4° C. Five times washing (300 μL/well) was performed with washing buffer (0.05% Tween 20 prepared PBS buffer) next day. Blocking step was started with 200 μL/well blocking buffer (5% horse serum prepared in washing buffer) at 37° C. for 2 h. 100 μL of formulations or tissue samples of IgG-Pep-9 and IgG were added to each well after aspiration of blocking buffer and then incubated at 4° C. for overnight. Ten times washing (300 L/well) was performed to each well before the 1 h, at 37° C. incubation of HRP-secondary antibody (Rabbit anti-mouse HRP). 100 μL of TMB was added to incubate at 37° C. for 15˜30 min after 5 times washing. 100 μL of 2M sulfuric acid was used to stop the HRP-TMB colorimetric reaction. Absorbance at 450 nm was measured for each well. IgG-Pep-9 and free IgG molecule formulation were diluted with blocking buffer to establish the quantification curve. Tissue samples (including blood serum, brain parenchyma, brain capillary) were diluted as needed to fall in the linear quantitation range.

TABLE A
Script S1 Matlab script for calculating the mean diffusivity of FAM-
labeled peptides in Matrigel embedded in the microfluidic chamber.

```
%% Read data
%0.6166pixel/um
clear all
files=dir(‘FAM−_Time Lapse_20180416_01_*.tif’);
num=length(files);
for i=1:num
  Iraw_Pep1(:,:,i) = imread(files(i).name);
end
files=dir(‘FAM−_Time Lapse_20180416_02_*.tif’);
num=length(files);
for i=1:num
  Iraw_Pep3(:,:,i) = imread(files(i).name);
end
files=dir(‘FAM−_Time Lapse_20180416_03_*.tif’);
num=length(files);
for i=1:num
  Iraw_Pep5(:,:,i) = imread(files(i).name);
end
files=dir(‘FAM−_Time Lapse_20180416_04_*.tif’);
num=length(files);
for i=1:num
  Iraw_R9(:,:,i) = imread(files(i).name);
end
files=dir(‘FAM−_Time Lapse_20180416_05_*.tif’);
num=length(files);
for i=1:num
  Iraw_CR7C(:,:,i) = imread(files(i).name);
end
files=dir(‘FAM−_Time Lapse_20180416_06_*.tif’);
num=length(files);
for i=1:num
  Iraw_Angiopep2(:,:,i) = imread(files(i).name);
end
Iraw_Pep1=Iraw_Pep1(1:13105,1:1993,:);
Iraw_Pep3=Iraw_Pep3(1:13105,1:1993,:);
Iraw_Pep5=Iraw_Pep5(1:13105,1:1993,:);
Iraw_R9=Iraw_R9(1:13105,1:1993,:);
Iraw_CR7C=Iraw_CR7C(1:13105,1:1993,:);
Iraw_Angiopep2=Iraw_Angiopep2(1:13105,1:1993,:);
%% Blocking
for j=1:num
  Pep1(:,j) = sepblockfun(Iraw_Pep1(:,:,j),[5, 1993],@mean); % 10 pixel (30.83 um) section
  Pep3(:,j) = sepblockfun(Iraw_Pep3(:,:,j),[5, 1993],@mean);
  Pep5(:,j) = sepblockfun(Iraw_Pep5(:,:,j),[5, 1993],@mean);
  R9(:,j) = sepblockfun(Iraw_R9(:,:,j),[5, 1993],@mean);
  CR7C(:,j) = sepblockfun(Iraw_CR7C(:,:,j),[5, 1993],@mean);
  Angiopep2(:,j) = sepblockfun(Iraw_Angiopep2(:,:,j),[5, 1993],@mean);
end
%% Image colormap
figure(1);
  subplot(1,3,1); imagesc(Pep1(:,1)); title(‘0hr’);
  subplot(1,3,2); imagesc(Pep1(:,11)); title(‘5hr’);
  subplot(1,3,3); imagesc(Pep1(:,25)); title(‘12hr’);
figure(2);
  subplot(1,3,1); imagesc(Pep3(:,1)); title(‘0hr’);
  subplot(1,3,2); imagesc(Pep3(:,11)); title(‘5hr’);
  subplot(1,3,3); imagesc(Pep3(:,25)); title(‘12hr’);
figure(3);
  subplot(1,3,1); imagesc(Pep5(:,1)); title(‘0hr’);
  subplot(1,3,2); imagesc(Pep5(:,11)); title(‘5hr’);
  subplot(1,3,3); imagesc(Pep5(:,25)); title(‘12hr’);
figure(4);
  subplot(1,3,1); imagesc(R9(:,1)); title(‘0hr’);
  subplot(1,3,2); imagesc(R9(:,11)); title(‘5hr’);
  subplot(1,3,3); imagesc(R9(:,25)); title(‘12hr’);
figure(5);
  subplot(1,3,1); imagesc(CR7C(:,1)); title(‘0hr’);
  subplot(1,3,2); imagesc(CR7C(:,11)); title(‘5hr’);
  subplot(1,3,3); imagesc(CR7C(:,25)); title(‘12hr’);
figure(6);
  subplot(1,3,1); imagesc(Angiopep2(:,1)); title(‘0hr’);
  subplot(1,3,2); imagesc(Angiopep2(:,11)); title(‘5hr’);
  subplot(1,3,3); imagesc(Angiopep2(:,25)); title(‘12hr’);
%% Figure out start of diffusion (0 position)
C1=diff(Pep1);
[temp1,pos1] = sort(C1, ‘ascend’);
C2=diff(Pep3);
[temp2, pos2] = sort(C2, ‘ascend’);
C3=diff(Pep5);
[temp3,pos3] = sort(C3, ‘ascend’);
C4=diff(R9);
[temp4, pos4] = sort(C4, ‘ascend’);
C5=diff(CR7C);
[temp5,pos5] = sort(C5, ‘ascend’);
C6=diff(Angiopep2);
[tem6, pos6] = sort(C6, ‘ascend’);
num1=pos1(1)+1;
num2=pos2(1)+1;
num3=pos3(1)+1;
num4=pos4(1)+1;
num5=pos5(1)+1;
num6=pos6(1)+1;
%% From 0 position to end
Pep1_n=Pep1(num1:end,:,:);
Pep3_n=Pep3(num2:end,:,:);
Pep5_n=Pep5(num3:end,:,:);
R9_n=R9(num4:end,:,:);
CR7C_n=CR7C(num5:end,:,:);
Angiopep2_n=Angiopep2(num6:end,:,:);
%% Image
figure(7);
  subplot(1,3,1); imagesc(Pep1_n(:,1)); title(‘0hr’);
  subplot(1,3,2); imagesc(Pep1_n(:,11)); title(‘5hr’);
  subplot(1,3,3); imagesc(Pep1_n(:,25)); title(‘12hr’);
figure(8);
  subplot(1,3,1); imagesc(Pep3_n(:,1)); title(‘0hr’);
  subplot(1,3,2); imagesc(Pep3_n(:,11)); title(‘5hr’);
  subplot(1,3,3); imagesc(Pep3_n(:,25)); title(‘12hr’);
figure(9);
  subplot(1,3,1); imagesc(Pep5_n(:,1)); title(‘0hr’);
  subplot(1,3,2); imagesc(Pep5_n(:,11)); title(‘5hr’);
  subplot(1,3,3); imagesc(Pep5_n(:,25)); title(‘12hr’);
figure(10);
  subplot(1,3,1); imagesc(R9_n(:,1)); title(‘0hr’);
  subplot(1,3,2); imagesc(R9_n(:,11)); title(‘5hr’);
  subplot(1,3,3); imagesc(R9_n(:,25)); title(‘12hr’);
figure(11);
  subplot(1,3,1); imagesc(CR7C_n(:,1)); title(‘0hr’);
  subplot(1,3,2); imagesc(CR7C_n(:,11)); title(‘5hr’);
  subplot(1,3,3); imagesc(CR7C_n(:,25)); title(‘12hr’);
figure(12);
  subplot(1,3,1); imagesc(Angiopep2_n(:,1)); title(‘0hr’);
  subplot(1,3,2); imagesc(Angiopep2_n(:,11)); title(‘5hr’);
  subplot(1,3,3); imagesc(Angiopep2_n(:,25)); title(‘12hr’);
%% Normalize
d=[0:1100];
d=d*5*0.6166;
x1=find(d>832,1);
x2=find(d>1000,1);
dist=d(x1:x2);
normPep1=zeros(x2−x1+1,num);
normPep3=zeros(x2−x1+1,num);
normPep5=zeros(x2−x1+1,num);
normR9=zeros(x2−x1+1,num);
normCR7C=zeros(x2−x1+1,num);
normAngiopep2=zeros(x2−x1+1,num);
for m=1:num
  Pep1_norm=Pep1_n(x1:x2,:);
  normPep1(:,m)=Pep1_norm(:,m)−min(Pep1_norm(:,m));
  normPep1(:,m)=normPep1(:,m)./max(normPep1(:,m));
  Pep3_norm=Pep3_n(x1:x2,:);
  normPep3(:,m)=Pep3_norm(:,m)−min(Pep3_norm(:,m));
  normPep3(:,m)=normPep3(:,m)./max(normPep3(:,m));
  Pep5_norm=Pep5_n(x1:x2,:);
  normPep5(:,m)=Pep5_norm(:,m)−min(Pep5_norm(:,m));
  normPep5(:,m)=normPep5(:,m)./max(normPep5(:,m));
  R9_norm=R9_n(x1:x2,:);
  normR9(:,m)=R9_norm(:,m)−min(R9_norm(:,m));
  normR9(:,m)=normR9(:,m)./max(normR9(:,m));
  CR7C_norm=CR7C_n(x1:x2,:);
  normCR7C(:,m)=CR7C_norm(:,m)−min(CR7C_norm(:,m));
  normCR7C(:,m)=normCR7C(:,m)./max(normCR7C(:,m));
  Angiopep2_norm=Angiopep2_n(x1:x2,:);
  normAngiopep2(:,m)=Angiopep2_norm(:,m)−min(Angiopep2_norm(:,m));
  normAngiopep2(:,m)=normAngiopep2(:,m)./max(normAngiopep2(:,m));
end
%% image
for k=1:10:num
  hold on;
  figure(13);
  plot(dist,normPep1(:,k));
  title(‘Pep1’)
  xlabel(‘Distance (um)’);
  ylabel(‘Intensity’);
  legend(‘0hr’,‘5hr’,‘10hr’);
end
hold off;
for k=1:10:num
  hold on;
  figure(14);
  plot(dist,normPep3(:,k));
  title(‘Pep3’)
  xlabel(‘Distance (um)’);
  ylabel(‘Intensity’);
  legend(‘0hr’,‘5hr’,‘10hr’);
end
hold off;
for k=1:10:num
  hold on;
  figure(15);
  plot(dist,normPep5(:,k));
  title(‘Pep-5’)
  xlabel(‘Distance (um)’);
  ylabel(‘Intensity’);
  legend(‘0hr’,‘5hr’,‘10hr’);
end
hold off;
for k=1:10:num
  hold on;
  figure(16);
  plot(dist,normR9(:,k));
  title(‘R9’)
  xlabel(‘Distance (um)’);
  ylabel(‘Intensity’);
  legend(‘0hr’,‘5hr’,‘10hr’);
end
hold off;
for k=1:10:num
  hold on;
  figure(17);
  plot(dist,normCR7C(:,k));
  title(‘CR7C’)
  xlabel(‘Distance (um)’);
  ylabel(‘Intensity’);
  legend(‘0hr’,‘5hr’,‘10hr’);
end
hold off;
for k=1:10:num
  hold on;
  figure(18);
  plot(dist,normAngiopep2(:,k));
  title(‘Angiopep2’)
  xlabel(‘Distance (um)’);
  ylabel(‘Intensity’);
  legend(‘0hr’,‘5hr’,‘10hr’);
end
hold off;
%% Curve fitting nlinfit
global t
t=1:25;
% X: dist,t
fun=@(D,dist) erfc(dist/(2*sqrt(D)))’;
Dguess=1;
D=nlinfit(dist, normR9(:,1),fun, Dguess);
```

### Example 5—Results

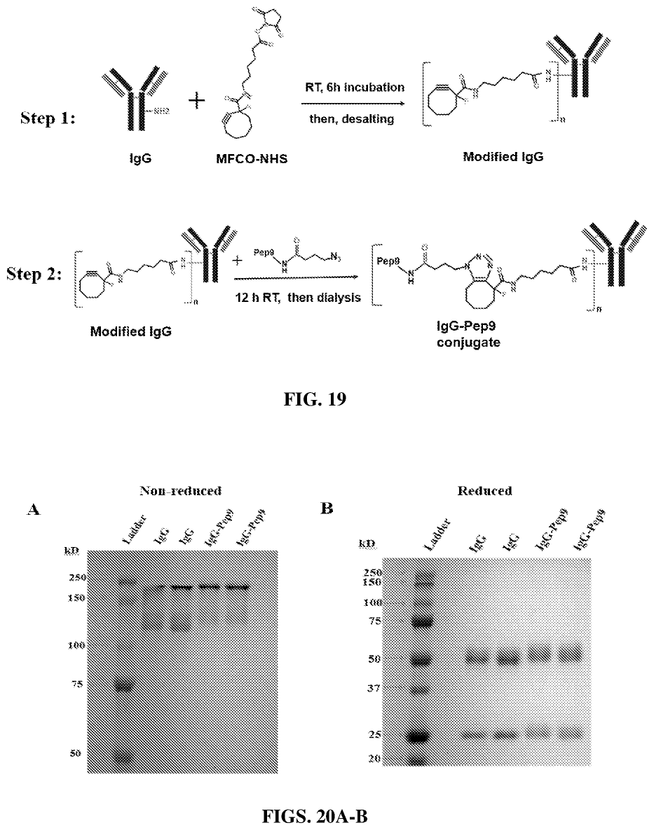

Characterization of IgG-Pep-9 conjugate formulation. Pep-9 (CLWRPAADC (SEQ ID NO: 27)) was discovered by our lab from biopanning with M13 phage display library against in vitro BBB model. Pep-9 is able to transport the in vitro and in vivo BBB models which have been validated in our past studies. Here, Pep-9 was modified to conjugate with IgG molecule by copper-free click chemistry in order to improve the delivery of macromolecules into the brain. Generic anti-mouse CTLA-4 antibody was selected as the model IgG molecule. There were two steps of conjugation by utilizing copper-free click chemistry, as demonstrated in FIG. 19, at step 1, the IgG molecules were modified by monofluoro-substituted cyclooctyne (MFCO)-n-hydroxysuccinimide ester (NHS) and then were purified by a desalting column. Thereafter, InVivoPure dilution buffer (pH=7.0) was used to buffer-exchanged to the desalting-purified IgG samples; at step 2, modified IgG molecules were conjugated with Pep-9 by click chemistry; Pep-9 was synthesized by Fmoc chemistry, additional lysine residue was introduced to Pep-9 sequence at N-terminus, and then azide (N3) group was coupled at lysine residue. With 12 h incubation, azide group of modified Pep-9 specifically conjugated to cyclooctyne of modified IgG molecule by copper-free click chemistry (FIG. 19). Dialysis approach (MWCO 50K) was utilized to purify the IgG-Pep-9 conjugate formulation.

The concentration of IgG or IgG-Pep-9 formulation was determined by the absorbance at 280 nm with respective extinction coefficient (Beer's law, 6=13.3 for anti-mouse CTLA-4 antibody from BioXcell). The mean recovery of the IgG-Pep-9 after formulation development was 57.4%, which was calculated from the concentration and respective volume of IgG and IgG-Pep-9 formulations before and after click chemistry.

The success of IgG and Pep-9 coupling was confirmed by SDS-PAGE, formulations with both non-reduced and reduced pretreatment, data were shown in FIGS. 20A-B. Each IgG molecule commonly has molecular weight ˜150 kD, while reduced IgG molecule dissociates into light chains (˜25 kD) and heavy chains (˜50 kD). Here anti-mouse CTLA-4 antibody as the IgG molecule has the molecular weight˜150 kD, while the IgG-Pep-9 conjugate formulation demonstrated the slight increase in size, as shown in FIG. 20A. The reduced SDS-PAGE, data were indicated in FIG. 20B, there was an apparent increase in size of light chains and heavy chains of reduced IgG-Pep-9 formulation compared to reduced free IgG molecule. As the qualification method, SDS-PAGE can't indicate the molecular ratio of IgG to Pep-9 of the IgG-Pep-9 conjugate formulation.

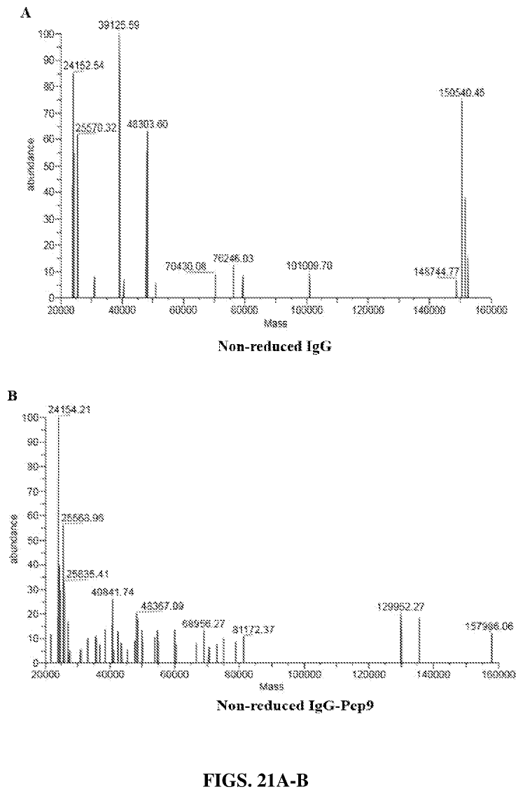

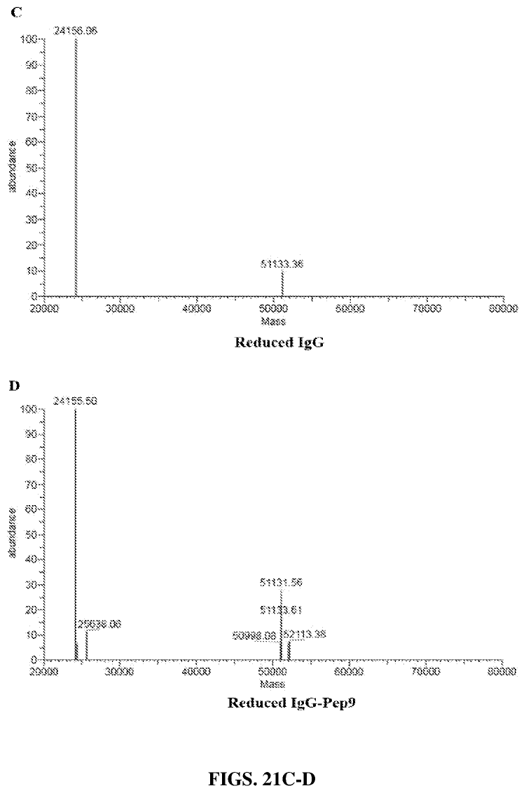

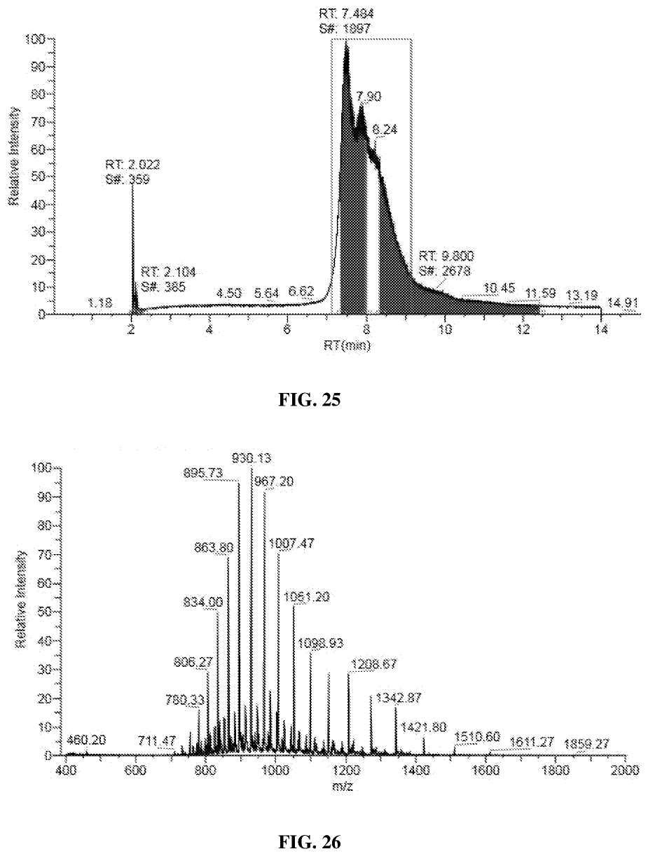

LC-MS method was developed to confirm the molecular weight of free IgG or IgG-Pep-9 formulations in non-reduced and reduced conditions. All the formulations were eluted from protein microtrap column and analyzed by Electrospray Ionization-Ion Trap-Mass Spectrometry (ESI-IT-MS). The mass spectra were all deconvolved spectrum in FIGS. 21A-D, which were calculated from individual total ion chromatogram (TIC) (FIG. 25) and selected dominant mass spectrum (m/z) from TIC (FIG. 26) of each formulation. In non-reduced condition, the mass associated with anti-mouse-CTLA-4 antibody (IgG) was 150,540.45 Dalton (D), which was demonstrated from the deconvolved mass spectrum in FIG. 21A, while the other mass species were the impurity components from IgG production line or from the fragmentation of IgG molecule during the procedures of formulation development. In FIG. 21B, the dominant mass for IgG-Pep-9 was 157986.06 D which indicated that five of Pep-9 molecules were conjugated to each IgG molecule (the mass for each Pep-9 molecule plus linker was ˜1483 D); meanwhile, many impurity species were present in the final IgG-Pep-9 formulation. Majority impurity components from the non-reduced IgG-Pep-9 formulation was inherited from the original antibody from the vendor, which can be told by the overlap of impurity species in FIGS. 21A-B. In reduced condition, 50 mM DTT pretreatment for 10 min at 70° C., anti-mouse-CTLA-4 antibody (IgG) was dissociated into two main mass species 24,156.06 and 51,133.33 D, which were correlated with the light chain and heavy chain of IgG 25 respectively (FIG. 21C). Reduced IgG-Pep-9 formulation showed multiple mass species in FIG. 21D, the dominant mass 25,638.06 D was the IgG-Pep-9 light chain with one Pep-9 molecule conjugation, while 24,155.50 D was the light chain of free IgG molecule. There was heterogeneous distribution in the heavy chain mass species of reduced IgG-Pep-9 formulation, which corrected with isotope effect and possibly one Pep-9 molecule coupling.

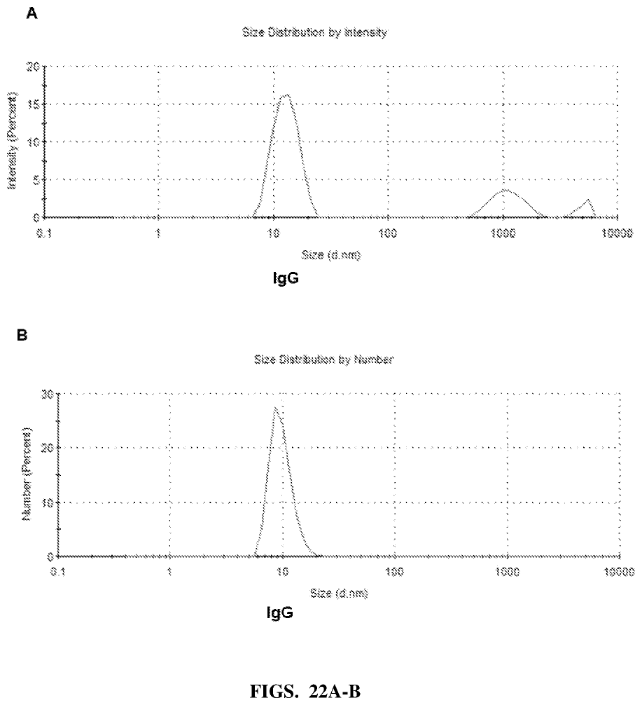

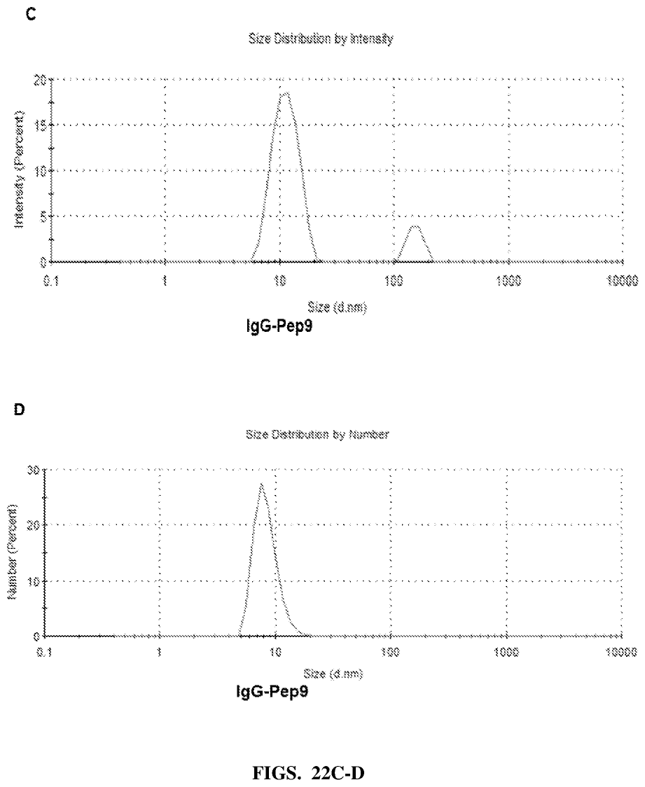

DLS size measurement was performed to the IgG and IgG-Pep-9 formulations to detect the aggregate species in the samples. The size quantitation range for Malvern Zetasizer is 1 nm˜3,000 nm. IgG and IgG-Pep-9 molecules were both around 10-15 nm by intensity and by molecular number distribution (FIGS. 22A-D). The aggregate was 25.2% (FIG. 22A) and 11.6% (FIG. 22C) by intensity percent in IgG and IgG-Pep-9 formulation, respectively. However, by molecular number (percent), it had 0.2% and 0% aggregate in the IgG (FIG. 22B) and IgG-Pep-9 (FIG. 22D) formulation respectively.

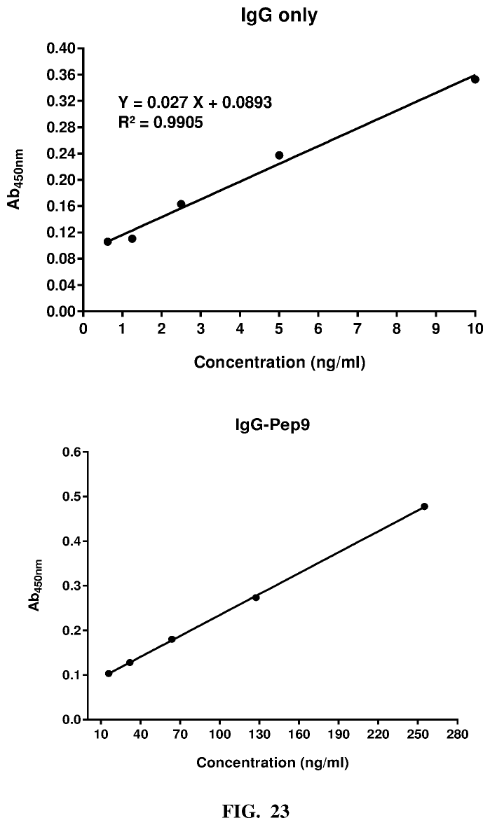

Quantification of IgG-Pep-9 formulation. Linear quantification range of IgG and IgG-Pep-9 formulations were determined by Enzyme-linked immunosorbent assay (ELISA). It turned out that when plotting absorbance at 450 nm(_{AM450 nm)} of ELISA assay (Y) versus the concentrations of IgG (X), Y=0.027 X+0.0893, R²=0.9905, the linear quantification range was 0.625˜10 ng/ml. For IgG-Pep-9 formulation, ELISA quantification range was 15.9-255 ng/ml, Y=0.0016X+0.078, R²=0.9998, the standard curves were demonstrated in FIG. 23. Here the IgG molecule was anti-mouse-CTLA-4 antibody as well. In order to accurately quantify the concentration of IgG and IgG-Pep-9 molecules in all the samples, both in formulations and biospecimen, it is required to dilute the samples to fall in the linear quantification range as needed.

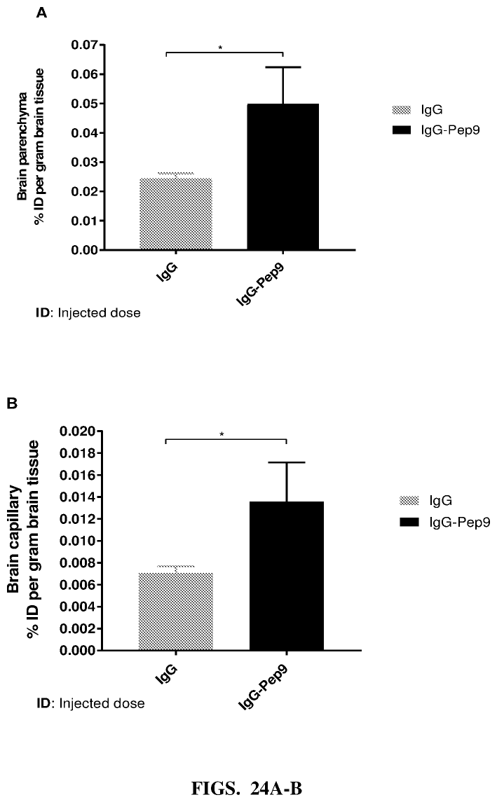

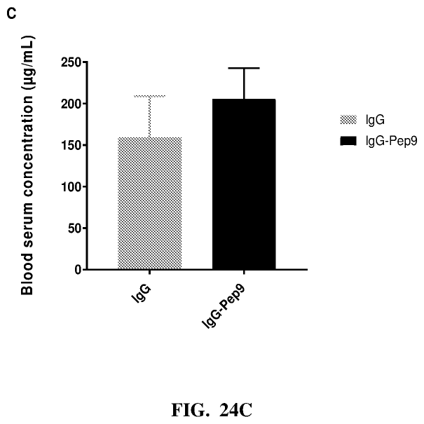

In vivo delivery of IgG-Pep-9 formulation. IgG-Pep-9 and free IgG formulations were delivered intravenously at the tail vein in two separate groups of Balb/C mice, IgG-Pep-9 and IgG groups, each group had 5 mice. The dosing regimen was 10 mg (IgG/IgG-Pep-9 formulation)/kg body weight, single dose. After 24 h circulation, blood was collected, perfusion was performed to have vascular washout in the brain, then the whole brain was dissected from each mouse. Capillary depletion (Triguero et al., 1990) was conducted accordingly to separate the brain parenchyma (brain tissue) from the brain capillary by dextran-driven density gradient centrifugation. Blood serum from both dosing treatment group was diluted at a dilution factor of 10⁴ ˜10⁵ before ELISA quantification assay. Brain parenchyma and brain capillary fractions from brain homogenate were quantified directly with developed ELISA assay without dilution, while the total injection dose and brain weight were counted to normalize the brain distribution. IgG-Pep-9 conjugate formulation had mean brain parenchyma distribution 0.050% of injection dose (ID) per gram brain weight, which was significantly higher than 0.024% of ID per gram brain weight in IgG group with P<0.05 (FIG. 24A). The brain capillary distribution difference between IgG-Pep-9 and IgG groups also had statistical significance, P<0.05, with the mean value 0.014 and 0.007% of injection dose (ID) per gram brain weight for IgG-Pep-9 and IgG respectively (FIG. 24B). The brain parenchyma distribution of IgG or IgG-Pep-9 formulation indicated that those macromolecules transported BBB and deposited into the brain tissue, while the brain capillary distribution represented those IgG or IgG-Pep-9 molecules still bound or internalized in the brain capillary endothelial cells and hadn't go across the BBB during the kinetics of BBB transport. Therefore, in IgG-Pep-9 group, 24 h after intravenous dosing, around 78% (0.05/(0.05+0.014)) of IgG-Pep-9 molecules that bound to the BBB had successfully transported BBB and delivered into the brain; while in IgG group, the ratio was 77%. The blood serum concentration of IgG and IgG-Pep-9 in the respective experimental groups were quantified and shown in FIG. 24C, while the blood serum distribution in IgG-Pep-9 group was slightly higher than IgG group, but without statistical significance.

### Example 6—Discussion

Antibodies (IgG) as the macromolecules, only around 0.1% (brain/blood concentration) can deliver to the brain parenchyma for the existence of BBB (Abbott et al., 2010). Molecular trojan horse technology is the most popular non-invasive approach to transport BBB and delivery the IgG molecule to the brain. Pep-9 (CLWRPAADC (SEQ ID NO: 27)), a cysteine-constraint cyclic peptide discovered previously by our group was selected as the peptide trojan horse to transport IgG into the brain. Here, Copper-free click chemistry was used to couple Pep-9 and IgG molecules by strain-promoted azide-alkyne cycloaddition (SPAAC) reaction (scheme in FIG. 19) (Regina et al., 2015b; Takayama et al., 2019). Copper-free click chemistry has been widely used to modify the biomolecules in drug delivery and cell engineering field for the reason that it is high yield, low cytotoxicity, irreversible reaction under physiological conditions (Takayama et al., 2019). The anti-mouse CTLA-4 antibody (from BioXcell) as the model IgG molecule in our study was produced in hybridoma and underwent one-step protein A purification. To minimize the influence of host cell proteins (HCPs) impurity (Rane et al., 2019) from hybridoma in the original IgG formulation, a control IgG group was prepared and processed through two steps conjugation as IgG-Pep-9 group. The goal for this study design is to have similar HCPs impurity background in the developed and purified IgG-Pep-9 formulation as in the free IgG formulation. We conducted a serial rounds of formulation optimization, which aimed to have high yield of IgG-Pep-9 conjugation, low degradation caused by HCPs impurity and low aggregation happen during formulation development. Based on these concern, desalting and dialysis were eventually chosen as the purification methods at each step of IgG-Pep-9 formulation development.

SDS-PAGE combined with Mass Spectrometry have been frequently used to characterize the size and molecular weight of the IgG molecules (Kirley et al., 2018; Nebija et al., 2011; Rathnayaka et al., 2018). One IgG molecule (˜150 kD) as demonstrated in FIG. 1, has two identical light chains and heavy chains. Under reducing condition (DTT), one anti-mouse CTLA-4 antibody molecule dissociates into two light chains (˜24,156 D) and two heavy chains (˜51,133 D).While, the data of SDS-PAGE and LC-MS agreed about the size estimation of non-reduced and reduced IgG or IgG-Pep-9 molecules (FIGS. 20A-B and 21A-D). The impurity come from HCPs or degradation of the IgG from two steps of conjugation and purification were demonstrated in the smear bands below 150 kD of SDS-PAGE (FIG. 20A), and in the heterogenous mass species of deconvolved mass spectrum (FIGS. 21A-B). Even though, the majority component in the IgG and IgG-Pep-9 formulations were the IgG molecules with and without Pep-9 (FIGS. 20A-B). In deconvolved mass spectrum, there was a mass species (157,986 D) that indicate 1:5 ratio of IgG and Pep-9 molecule in the non-reduced IgG-Pep-9 formulation (FIG. 21B); while in reduced IgG-Pep-9 formulation, only 25,638 D (ratio 1:1, light chain: Pep-9) and 52,113D (potential ratio 1:1, heavy chain:Pep-9) presented indicated the conjugation of IgG and Pep-9 (FIG. 21D). The discrepancy of conjugation ratio between IgG and Pep-9 molecules in non-reduced and reduced IgG-Pep-9 formulation comes from the following factors: (1) heterogeneity of mass abundance in the deconvolved spectrum in both non-reduced and reduced IgG-Pep-9 formulation; (2) low resolution of ESI-IT-MS for intact IgG molecule (Najdekr et al., 2016); (3) the influence of the mass species associated with impurity from the original IgG formulation. In order to accurately estimate the molecular weight and conjugation ratio of the formulations, intact IgG or IgG-Pep-9 molecules can be digested by the peptidase (e.g., trypsin) and analyzed by triple quadrupole MS/MS by proteomics approach.

Aggregation is one of the critical quality attributes (CQA) of antibody product, which impacts the safety and efficacy of IgG product (Fisher et al., 2016). It has been found that IgG aggregate can generate immunogenicity and bring the function loss to the IgG molecule. To prevent the unexpected immunogenicity from IgG or IgG-Pep-9 aggregate in mice model, formulation development has been optimized to minimize the aggregation. DLS was employed to characterize the size distribution and especially detect the aggregate distribution in final purified IgG-Pep-9/IgG formulations. Size distribution by intensity is primary obtained by DLS, and sensitive to presence of the large molecules. Number-weighted size distribution is derived from intensity-weighted size distribution by mie theory. The size estimation of IgG and IgG-Pep-9 by intensity-based and number-based DLS was aligned with each other (FIGS. 22A-D). Size-weighted size distribution, the percent calculation of each size species has bias for the large molecule, the intensity of a large molecule is 10⁶ times of a small molecule (Stetefeld et al., 2016). Hence the percent aggregation reported in FIG. 22A and FIG. 22C was overestimated. The number-based percent value in FIG. 22B and FIG. 22D represented the distribution of actual molecular numbers, which demonstrated that there was neglectable numbers of aggregate molecules in IgG and IgG-Pep-9 formulation. It proved that the IgG-Pep-9 we developed don't have the aggregation issue.

ELISA assay as an affinity-based assay was developed to quantify the IgG or IgG-Pep-9 concentration in the tissue samples from in vivo study. The output of ELISA replies on the strong binding between IgG and its respective antigen. The linear range we obtained from ELISA assay of IgG and IgG-Pep-9 formulations (FIG. 23) indicated that there was significant binding affinity loss between IgG-Pep-9 and the antigen (mouse CTLA-4 protein). The compromise of binding affinity in IgG-Pep-9 formulation was due to Pep-9 binding to the Fab region of IgG molecule, which indicated in the LC-MS results (FIGS. 21A-D). It is known that Fab region of an IgG molecule mediate the binding of the IgG to its own antigen (Stetefeld et al., 2016). CTLA-4 protein (antigen of our tested IgG molecule) is expressed on brain CD8⁺ T cell, but not on BBB (Smolders et al., 2018). The scope of our study is to deliver IgG-Pep-9 into the brain by Pep-9-mediated BBB transport, the binding affinity loss of IgG-Pep-9 to CTLA-4 protein can't impact the brain delivery of the formulation in vivo.

In vivo delivery study of IgG and IgG-Pep-9 formulations proved that BBB shuttle peptide Pep-9 we discovered can improve the IgG delivery into the brain (FIGS. 24A-C). IgG-Pep-9 formulation had two-fold enhancement of distribution in brain parenchyma and brain capillary compared to free IgG formulation. Endogenous and exogenously administered IgG uptake into the brain capillary endothelial cell (BBB) by limited endocytosis pathway. Majority endocytosed IgG molecules recycle back to blood vessel following the pathway mediated by neonatal Fc receptor (FcRn); meanwhile, some fraction of endocytosed IgG molecules degrade when trafficking to the lysosomes of endothelial cells, pericytes also involve in upregulation of lysosomal degradation of IgG (Chang et al., 2018; Villasenõr et al., 2016). Therefore, IgG delivery into the brain parenchyma is extremely low, 0.1% endogenous IgG in human plasma can reach to the brain tissue. Our peptide Pep-9 can transport BBB by binding to unknown target on the BBB (Peng et al., ChemRxiv 2019). The Pep-9 binding target-mediated BBB transport improve the delivery of IgG molecules into the brain parenchyma and capillary. The circulation kinetics of IgG molecules was not affected by the Pep-9 molecule coupling, which was analyzed by the fact that after 24 h circulation, both formulations have 77% and 78% of molecules that bound to the capillaries that eventually delivered to the brain parenchyma.

As a proof of concept study, we wanted to validate if previously discovered BBB peptide shuttle Pep-9 is able to improve the IgG delivery into the brain. However, there were challenges in our study, future work will focus on improvement of the conjugation strategy between Pep-9 and IgG (e.g., fusion protein or site-specific chemistry), developing IgG-Pep-9 formulation by using high purify and quality of IgG products (e.g., choosing therapeutic antibody). Moreover, Pep-9 will used to couple with other biologic molecules (e.g., protein, enzyme) to explore the potential of Pep-9 as the carriers to transport macromolecules into the brain.

In conclusion, BBB peptide shuttle Pep-9 was successfully conjugated with IgG molecules by copper-free click chemistry. A series of methods (SDS-PAGE, LC-MS, DLS) were developed to characterize the IgG-Pep-9 and free IgG formulations before in vivo delivery study. IgG-Pep-9 formulation demonstrated improved brain distribution than free IgG formulation in vivo, which was determined by the sensitive ELISA method. Here, it proved that Pep-9 can ferry macromolecule into the brain.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.

### Examples 1-3

(1) Tarun Dua, Marco Garrido Cumbrera, Colin Mathers, S. S. Global Burden of Neurological Disorders: Estimates and Projections. In NEUROLOGICAL DISORDERS public health challenges; 2007; pp 27-39.
(2) Warren, K. E. Beyond the Blood:Brain Barrier: The Importance of Central Nervous System (CNS) Pharmacokinetics for the Treatment of CNS Tumors, Including Diffuse Intrinsic Pontine Glioma. Front. Oncol. 2018, 8 (Jul.), 1-11.
(3) Nicholson, C.; Hrabetova, S. Brain Extracellular Space: The Final Frontier of Neuroscience. Biophys. J. 2017, 113 (10), 2133-2142.
(4) Banks, W. A. From Blood-Brain Barrier to Blood Brain Interface:New Opportunities for CNS Drug Delivery. Nat. Rev. Drug Discov. 2016, 275-292.
(5) Pardridge, W. M. Drug Transport across the Blood-Brain Barrier. J. Cereb. Blood Flow Metab. 2012, 32 (11), 1959-1972.
(6) Abbott, N. J.; Patabendige, A. A. K.; Dolman, D. E. M.; Yusof, S. R.; Begley, D. J. Structure and Function of the Blood-Brain Barrier. Neurobiol. Dis. 2010, 37 (1), 13-25.
(7) Miller, D. S. Regulation of ABC Tramsporters at the Blood Brain Barrier. Clin. Pharmacol. Ther. 2015, 97 (4), 395-403.
(8) Strazielle, N.; Ghersi-Egea, J. F. Demonstration of a Coupled Metabolism-Efflux Process at the Choroid Plexus as a Mechanism of Brain Protection toward Xenobiotics. J. Neurosci. 1999, 19 (15), 6275-6289.
(9) Fong, C. W. Permeability of the Blood-Brain Barrier: Molecular Mechanism of Transport of Drugs and Physiologically Important Compounds. J. Membr. Biol. 2015, 248 (4), 651-669.
(10) Carter, P. J.; Lazar, G. A. Next Generation Antibody Drugs: Pursuit of the “High-Hanging Fruit.” Nat. Rev. Drug Discov. 2018, 17 (3), 197-223.
(11) Pardridge, W. M.; Boado, R. J. Reengineering Biopharmaceuticals for Targeted Delivery across the Blood-Brain Barrier, 1st ed.; Elsevier Inc., 2012; Vol. 503.
(12) Pardridge, W. M. Drug Transport across the Blood-Brain Barrier. J. Cereb. Blood Flow Metab. 2012, 32 (11), 1959-1972.
(13) Pardridge, W. M. The Blood-Brain Barrier: Bottleneck in Brain Drug Development. NeuroRx 2005, 2 (1), 3-14.
(14) Nicholson, C. Diffusion and Related Transport Mechanisms in Brain Tissue. Rep. Prog. Phys. 2001, 64, 815-884.
(15) Sykova, E.; Nicholson, C. Diffusion in Brain Extracellular Space. Physiol Rev. 2008, 88, 1277-1340.
(16) Novak, U.; Kaye, A. H. Extracellular Matrix and the Brain: Components and Function. J. Clin. Neurosci. 2000, 7 (4), 280-290.
(17) Wolak, D. J.; Thorne, R. G. Diffusion of Macromolecules in the Brain: Implications for Drug Delivery. Mol. Pharm. 2013, 10 (5), 1492-1504.
(18) Wolak, D. J.; Pizzo, M. E.; Thorne, R. G. Probing the Extracellular Diffusion of Antibodies in Brain Using in vivo Integrative Optical Imaging and Ex Vivo Fluorescence Imaging. J. Control. Release 2015, 10, 78-86.
(19) Thorne, R. G.; Nicholson, C. In vivo Diffusion Analysis with Quantum Dots and Dextrans Predicts the Width of Brain Extracellular Space. Proc. Natl. Acad. Sci. U.S.A 2006, 103 (14), 5567-5572.
(20) Hrabetova, S.; Hrabe, J.; Nicholson, C. Dead-Space Microdomains Hinder Extracellular Diffusion in Rat Neocortex during Ischemia. J. Neurosci. 2003, 23 (23), 8351-8359.
(21) Stalmans, S.; Bracke, N.; Wynendaele, E.; Gevaert, B.; Peremans, K.; Burvenich, C.; Polis, I.; De Spiegeleer, B. Cell-Penetrating Peptides Selectively Cross the Blood-Brain Barrier in vivo. PLoS One 2015, 10 (10), 1-22.
(22) Thorne, R. G.; Lakkaraju, A.; Rodriguez-Boulan, E.; Nicholson, C. In vivo Diffusion of Lactoferrin in Brain Extracellular Space Is Regulated by Interactions with Heparan Sulfate. Proc. Natl. Acad. Sci. U.S.A 2008, 105 (24), 8416-8421.
(23) Saito, R.; Krauze, M. T.; Noble, C. O.; Tamas, M.; Drummond, D. C.; Kirpotin, D. B.; Berger, M. S.; Park, J. W.; Bankiewicz, K. S. Tissue Affinity of the Infusate Affects the Distribution Volume during Convection-Enhanced Delivery into Rodent Brains: Implications for Local Drug Delivery. J. Neurosci. Methods 2006, 154 (1-2), 225-232.
(24) Nance, E. A.; Woodworth, G. F.; Sailor, K. A.; Shih, T.-Y.; Xu, Q.; Swaminathan, G.; Xiang, D.; Eberhart, C.; Hanes, J. Polymeric Nanoparticles Within Brain Tissue A Dense Poly(Ethylene Glycol) Coating Improves Penetration of Large A Dense Poly(Ethylene Glycol) Coating Improves Penetration of Large Polymeric Nanoparticles Within Brain Tissue. Sci. Transl. Med. 2012, 149 (119), 1-10.
(25) Medina, D. X.; Householder, K. T.; Ceton, R.; Kovalik, T.; Heffernan, J. M.; Shankar, R. V.; Bowser, R. P.; Wechsler-Reya, R. J.; Sirianni, R. W. Optical Barcoding of PLGA for Multispectral Analysis of Nanoparticle Fate in vivo. J. Control. Release 2017, 253, 172-182.
(26) Nance, E. A.; Woodworth, G. F.; Sailor, K. A.; Shih, T.; Swaminathan, G.; Xiang, D.; Eberhart, C. A Dense Poly(Ethylene Glycol) Coating Improves Penetration of Large Polymeric Nanoparticles within Brain Tissue. Sci. Transl. Med. 2012, 4 (149), 1-18.
(27) Khaitan, D.; Reddy, P. L.; Ningaraj, N. Targeting Brain Tumors with Nanomedicines: Overcoming Blood Brain Barrier Challenges. 2018, No. 67, 1-10.
(28) Neuwelt, E.; Abbott, N. J.; Abrey, L.; Banks, W. A.; Blakley, B.; Davis, T.; Engelhardt, B.; Grammas, P.; Nedergaard, M.; Nutt, J.; et al. Strategies to Advance Translational Research into Brain Barriers. Lancet Neurol. 2008, 7 (1), 84-96.
(29) Kovacs, Z. I.; Kim, S.; Jikaria, N.; Qureshi, F.; Milo, B.; Lewis, B. K.; Bresler, M.; Burks, S. R.; Frank, J. A. Disrupting the Blood-Brain Barrier by Focused Ultrasound Induces Sterile Inflammation. Proc. Natl. Acad. Sci. 2017, 114 (1), E75-E84.
(30) Carpentier, A.; Canney, M.; Vignot, A.; Reina, V.; Beccaria, K.; Horodyckid, C.; Karachi, C.; Leclercq, D.; Lafon, C.; Chapelon, J.-Y.; et al. Clinical Trial of Blood-Brain Barrier Disruption by Pulsed Ultrasound. Sci. Transl. Med. 2016, 8 (343), 343re2-343re2.
(31) Zhan, W.; Wang, C. H. Convection Enhanced Delivery of Chemotherapeutic Drugs into Brain Tumour. J. Control. Release 2018, 271 (November 2017), 74-87.
(32) Choi, C.; Kim, H. M.; Shon, J.; Park, J.; Kim, H. T.; Oh, S. H.; Kim, N. K.; Kim, O. J. Additional Increased Effects of Mannitol-Temozolomide Combined Treatment on Blood-Brain Barrier Permeability. Biochem. Biophys. Res. Commun. 2018, 497 (2), 769-775.
(33) Ghosh, D.; Peng, X.; Leal, J.; Mohanty, R. P. Peptides as Drug Delivery Vehicles across Biological Barriers. J. Pharm. Investig. 2018, 48 (1), 89-111.
(34) Diaz-Perlas, C.; Sanchez-Navarro, M.; Teixidó, M.; Giralt, E. Phage Display as a Tool to Discover BBB-Shuttle Peptides: Panning against a Human Blood-Brain Barrier Cellular Model. J. Pept. Sci. 2016, 22, 1-13.
(35) Lee, J. H.; Engler, J. A.; Collawn, J. F.; Moore, B. A. Receptor Mediated Uptake of Peptides That Bind the Human Transferrin Receptor. Eur. J. Biochem. 2001, 268 (7), 2004-2012.
(36) Malcor, J. D.; Payrot, N.; David, M.; Faucon, A.; Abouzid, K.; Jacquot, G.; Floquet, N.; Debarbieux, F.; Rougon, G.; Martinez, J.; et al. Chemical Optimization of New Ligands of the Low-Density Lipoprotein Receptor As Potential Vectors for Central Nervous System Targeting. J Med Chem 2012.
(37) Pardridge, W. M. Delivery of Biologics Across the Blood-Brain Barrier with Molecular Trojan Horse Technology. BioDrugs 2017, 31 (6), 503-519.
(38) Staquicini, F. I.; Ozawa, M. G.; Moya, C. A.; Driessen, W. H. P.; Barbu, E. M.; Nishimori, H.; Soghomonyan, S.; Flores, L. G.; Liang, X.; Paolillo, V.; et al. Systemic Combinatorial Peptide Selection Yields a Non-Canonical Iron-Mimicry Mechanism for Targeting Tumors in a Mouse Model of Human Glioblastoma. J. Clin. Invest. 2011, 121 (1), 161-173.
(39) Li, J.; Feng, L.; Fan, L.; Zhan, Y.; Guo, L.; Zhang, Q.; Chen, J.; Pang, Z.; Wang, Y.; Jiang, X.; et al. Targeting the Brain with PEG-PLGA Nanoparticles Modified with Phage-Displayed Peptides. Biomaterials 2011, 32 (21), 1301-1315.
(40) Li, J.; Zhang, Q.; Pang, Z.; Wang, Y.; Liu, Q.; Guo, L.; Jiang, X. Identification of Peptide Sequences That Target to the Brain Using in vivo Phage Display. Amino Acids 2012, 42 (6), 2373-2381.
(41) Ohtsuki, S.; Hirayama, M.; Lto, S.; Uchida, Y.; Tachikawa, M.; Terasaki, T. Quantitative Targeted Proteomics for Understanding the Blood—Brain Barrier: Towards Pharmacoproteomics. Expert Rev. Proteomics 2014, 11 (3), 303-313.
(42) Srivastava, A. S.; Kaido, T.; Carrier, E. Immunological Factors That Affect the in vivo Fate of T7 Phage in the Mouse. J. Virol. Methods 2004, 115 (1), 99-104.
(43) Deutscher, S. L. Phage Display in Molecular Imaging and Diagnosis of Canver. Chem. Rev. 2011, 110 (5), 3196-3211.
(44) Derda, R.; Tang, S. K. Y.; Li, S. C.; Ng, S.; Matochko, W.; Jafari, M. R. Diversity of Phage-Displayed Libraries of Peptides during Panning and Amplification. Molecules 2011, 16 (2), 1776-1803.
(45) Mitchell, D. J.; Steinman, L.; Kim, D. T.; Fathman, C. G.; Rothbard, J. B. Polyarginine Enters Cells More Efficiently than Other Polycationic Homopolymers. J. Pept. Res. 2000, 56 (5), 318-325.
(46) Regina, A.; Demeule, M.; Ché, C.; Lavallée, I.; Poirier, J.; Gabathuler, R.; Béliveau, R.; Castaigne, J.-P. Antitumour Activity of ANG1005, a Conjugate between Paclitaxel and the New Brain Delivery Vector Angiopep-2. Br. J. Pharmacol. 2008, 155 (2), 185-197.
(47) Demeule, M.; Regina, A.; Ché, C.; Poirier, J.; Nguyen, T.; Gabathuler, R.; Castaigne,
J.-P.; B6liveau, R. Identification and Design of Peptides as a New Drug Delivery System for the Brain. J. Pharmacol. Exp. Ther. 2008, 324 (3), 1064-1072.
(48) Regina, A.; Demeule, M.; Tripathy, S.; Lord-Dufour, S.; Currie, J.-C.; Iddir, M.; Annabi, B.; Castaigne, J.-P.; Lachowicz, J. E. ANG4043, a Novel Brain-Penetrant Peptide-MAb Conjugate, Is Efficacious against HER2-Positive Intracranial Tumors in Mice. Mol. Cancer Ther. 2015, 14 (1), 129-140.
(49) Sade, H.; Baumgartner, C.; Hugenmatter, A.; Moessner, E.; Freskgird, P. O.; Niewoehner, J. A Human Blood-Brain Barrier Transcytosis Assay Reveals Antibody Transcytosis Influenced by PH-Dependent Receptor Binding. PLoS One 2014, 9 (4), e96340.
(50) Raub, T. J.; Newton, C. R. Recycling Kinetics and Transcytosis of Transferrin in Primary Cultures of Bovine Brain Microvessel Endothelial Cells. J. Cell. Physiol. 1991, 149 (1), 1410151.
(51) Helms, H. C.; Abbott, N. J.; Burek, M.; Cecchelli, R.; Couraud, P.-O.; Deli, M. A.; Förster, C.; Galla, H. J.; Romero, I. A.; Shusta, E. V; et al. In vitro Models of the Blood-Brain Barrier: An Overview of Commonly Used Brain Endothelial Cell Culture Models and Guidelines for Their Use. J. Cereb. Blood Flow Metab. 2016, 36 (5), 862-890.
(52) Jafari, M. R.; Deng, L.; Kitov, P. I.; Ng, S.; Matochko, W. L.; Tjhung, K. F.; Zeberoff, A.; Elias, A.; Klassen, J. S.; Derda, R. Discovery of Light-Responsive Ligands through Screening of a Light-Responsive Genetically Encoded Library. ACS Chem. Biol. 2014, 9 (2), 443-450.
(53) Liu, G. W.; Livesay, B. R.; Kacherovsky, N. A.; Cieslewicz, M.; Lutz, E.; Waalkes, A.; Jensen, M. C.; Salipante, S. J.; Pun, S. H. Efficient Identification of Murine M2 Macrophage Peptide Targeting Ligands by Phage Display and next Generation Sequencing. Bioconjug. Chem. 2015, 26 (8), 1811-1817.
(54) Säll, A.; Sjöholm, K.; Waldemarson, S.; Happonen, L.; Karlsson, C.; Persson, H.; Malmström, J. Development of Phage-Based Antibody Fragment Reagents for Affinity Enrichment of Bacterial Immunoglobulin G Binding Proteins. J. Proteome Res. 2015, 14 (11), 4704-4713.
(55) Matochko, W. L.; Cory Li, S.; Tang, S. K. Y.; Derda, R. Prospective Identification of Parasitic Sequences in Phage Display Screens. Nucleic Acids Res. 2014, 42 (3), 1784-1798.
(56) Liu, G. W.; Livesay, B. R.; Kacherovsky, N. A.; Cieslewicz, M.; Lutz, E.; Waalkes, A.; Jensen, M. C.; Salipante, S. J.; Pun, S. H. Efficient Identification of Murine M2 Macrophage Peptide Targeting Ligands by Phage Display and Next-Generation Sequencing. Bioconjug. Chem. 2015, 26 (8), 1811-1817.
(57) Matochko, W. L.; Chu, K.; Jin, B.; Lee, S. W.; Whitesides, G. M.; Derda, R. Deep Sequencing Analysis of Phage Libraries Using Illumina Platform. Methods 2012, 58 (1), 47-55.
(58) Kyte, J.; Doolittle, R. F. A Simple Method for Displaying the Hydropathic Character of a Protein. J. Mol. Biol. 1982, 157 (1), 105-132.
(59) Rodi, D. J.; Soares, A. S.; Makowski, L. Quantitative Assessment of Peptide Sequence Diversity in M13 Combinatorial Peptide Phage Display Libraries. J. Mol. Biol. 2002, 322 (5), 1039-1052.
(60) He, B.; Tjhung, K. F.; Bennett, N. J.; Chou, Y.; Rau, A.; Huang, J.; Derda, R. Compositional Bias in Naïve and Chemically-Modified Phage-Displayed Libraries Uncovered by Paired-End Deep Sequencing. Sci. Rep. 2018, 8 (1), 1214.
(61) Yamaguchi, A.; Isozaki, K.; Nakamura, M.; Takaya, H.; Watanabe, T. Discovery of 12-Mer Peptides That Bind to Wood Lignin. Sci. Rep. 2016, 6 (Feb.), 1-11.
(62) Eigenmann, D. E.; Xue, G.; Kim, K. S.; Moses, A. V; Hamburger, M.; Oufir, M. Comparative Study of Four Immortalized Human Brain Capillary Endothelial Cell Lines, HCMEC/D3, HBMEC, TY10, and BB19, and Optimization of Culture. Fluids Barriers CNS 2013, 10 (33), 1-16.
(63) CL, H.; L, V. V.; B, R.; G, van K.; M, van G. DIPimage: A Scientific Image Processing Toolbox for MATLAB. Quantitative Imaging Group, Faculty of Applied Sciences, Delft University of Technology, Delft, The Netherlands. 1999.
(64) Liu, Y. L.; Perillo, E. P.; Liu, C.; Yu, P.; Chou, C. K.; Hung, M. C.; Dunn, A. K.; Yeh, H. C. Segmentation of 3D Trajectories Acquired by TSUNAMI Microscope: An Application to EGFR Trafficking. Biophys. J. 2016, 111 (10), 2214-2227.
(65) Trinczek, B.; Ebneth, A.; Mandelkow, E. M.; Mandelkow, E. Tau Regulates the Attachment/Detachment but Not the Speed of Motors in Microtubule-Dependent Transport of Single Vesicles and Organelles. J. Cell Sci. 1999, 112, 2355-2367.
(66) Ichikawa, T.; Yamada, M.; Homma, D.; Cherry, R. J.; Morrison, I. E. G.; Kawato, S. Digital Fluorescence Imaging of Trafficking of Endosomes Containing Low-Density Lipoprotein in Brain Astroglial Cells. Biochem. Biophys. Res. Commun. 2000, 269 (1), 25-30.
(67) de Bruin, K.; Ruthardt, N.; von Gersdorff, K.; Bausinger, R.; Wagner, E.; Ogris, M.; Bräuchle, C. Cellular Dynamics of EGF Receptor-Targeted Synthetic Viruses. Mol. Ther. 2007, 15 (7), 1297-1305.
(68) Daumas, F.; Destainville, N.; Millot, C.; Lopez, A.; Dean, D.; Salomé, L. Confined Diffusion without Fences of a G-Protein-Coupled Receptor as Revealed by Single Particle Tracking. Biophys. J. 2003, 84 (1), 356-366.
(69) Lee, S.-M.; Lee, E.-J.; Hong, H.-Y.; Kwon, M.-K.; Kwon, T.-H.; Choi, J.-Y.; Park, R.-W.; Kwon, T.-G.; Yoo, E.-S.; Yoon, G.-S.; et al. Targeting Bladder Tumor Cells In vivo and in the Urine with a Peptide Identified by Phage Display. Mol. Cancer Res. 2007, 5 (1), 11-19.
(70) Broda, E.; Mickler, F. M.; Lächelt, U.; Morys, S.; Wagner, E.; Bräuchle, C. Assessing Potential Peptide Targeting Ligands by Quantification of Cellular Adhesion of Model Nanoparticles under Flow Conditions. Journal of Controlled Release. 2015, pp 79-85.
(71) Park, S.; Kim, Y.-J.; Jon, S. A High-Affinity Peptide for Nicotinic Acetylcholine Receptor-A1 and Its Potential Use in Pulmonary Drug Delivery. J. Control Release 2014, 192, 141-147.
(72) Park, S.; Kim, Y.-J.; Jon, S. A High-Affinity Peptide for Nicotinic Acetylcholine Receptor-A1 and Its Potential Use in Pulmonary Drug Delivery. J. Control. Release 2014, 192, 141-147.
(73) Jin, W.; Qin, B.; Chen, Z.; Liu, H.; Barve, A.; Cheng, K. Discovery of PSMA-Specific Peptide Ligands for Targeted Drug Delivery. Int. J. Pharm. 2016, 513 (1-2), 138-147.
(74) Liu, H.; Funke, S. A.; Willbold, D. Transport of Alzheimer Disease Amyloid-Beta-Binding D-Amino Acid Peptides across an in vitro Blood-Brain Barrier Model. Rejuvenation Res. 2010, 13 (2-3), 210-213.
(75) Kurabi, A.; Pak, K. K.; Bernhardt, M.; Baird, A.; Ryan, A. F. Discovery of a Biological Mechanism of Active Transport through the Tympanic Membrane to the Middle Ear. Sci. Rep. 2016, 6 (22663), 1-12.
(76) Demeule, M.; Regina, A.; Ché, C.; Poirier, J.; Nguyen, T.; Gabathuler, R.; Castaigne, J.-P.; Béliveau, R. Identification and Design of Peptides as a New Drug Delivery System for the Brain. J. Pharmacol. Exp. Ther. 2008, 324 (3), 1064-1072.
(77) Herce, H. D.; Garcia, A. E.; Litt, J.; Kane, R. S.; Martin, P.; Enrique, N.; Rebolledo, A.; Milesi, V. Arginine-Rich Peptides Destabilize the Plasma Membrane, Consistent with a Pore Formation Translocation Mechanism of Cell-Penetrating Peptides. Biophys. J. 2009, 97 (7), 1917-1925.
(78) Melikov, K.; Hara, A.; Yamoah, K.; Zaitseva, E.; Zaitsev, E.; Chernomordik, L. V. Efficient Entry of Cell-Penetrating Peptide Nona-Arginine into Adherent Cells Involves a Transient Increase in Intracellular Calcium. Biochem. J. 2015, 471 (2), 221-230.
(79) Bertrand, Y.; Currie, J. C.; Demeule, M.; Regina, A.; Ché, C.; Abulrob, A.; Fatehi, D.; Sartelet, H.; Gabathuler, R.; Castaigne, J. P.; et al. Transport Characteristics of a Novel Peptide Platform for CNS Therapeutics. J. Cell. Mol. Med. 2010, 14 (12), 2827-2839.
(80) Drappatz, J.; Brenner, A.; Wong, E. T.; Eichler, A.; Schiff, D.; Groves, M. D.; Mikkelsen, T.; Rosenfeld, S.; Sarantopoulos, J.; Meyers, C. A.; et al. Phase I Study of GRN1005 in Recurrent Malignant Glioma. Clin. Cancer Res. 2013, 19 (6), 1567-1576.
(81) Demeule, M.; Beaudet, N.; Régina, A.; Besserer-Offroy, É.; Murza, A.; Tétreault, P.; Belleville, K.; Ché, C.; Larocque, A.; Thiot, C.; et al. Conjugation of a Brain-Penetrant Peptide with Neurotensin Provides Antinociceptive Properties. J. Clin. Invest. 2014, 124 (3), 1199-1213.
(82) Regina, A.; Demeule, M.; Tripathy, S.; Lord-Dufour, S.; Currie, J.-C.; Iddir, M.; Annabi, B.; Castaigne, J.-P.; Lachowicz, J. E. ANG4043, a Novel Brain-Penetrant Peptide-MAb Conjugate, Is Efficacious against HER2-Positive Intracranial Tumors in Mice. Mol. Cancer Ther. 2015, 14 (1), 129-140.
(83) Lim, S.; Kim, W. J.; Kim, Y. H.; Lee, S.; Koo, J. H.; Lee, J. A.; Yoon, H.; Kim, D. H.; Park, H. J.; Kim, H. M.; et al. DNP2 Is a Blood-Brain Barrier-Permeable Peptide Enabling CtCTLA-4 Protein Delivery to Ameliorate Experimental Autoimmune Encephalomyelitis. Nat. Commun. 2015, 6, 1-13.
(84) Jiao, C.; Delaroche, D.; Burlina, F.; Alves, I. D.; Sagan, S. Translocation and Endocytosis for Cell-Penetrating Peptide Internalization. J. Biol. Chem. 2009, 284 (49), 33957-33965.
(85) Kruth, H. S. Receptor-Independent Fluid-Phase Pinocytosis Mechanisms for Induction of Foam Cell Formtion with Native LDL Particles. Curr Opin Lipidol. 2011, 22 (5), 386-393.
(86) Jin, B.-J.; Smith, A. J.; Verkman, A. S. Spatial Model of Convective Solute Transport in Brain Extracellular Space Does Not Support a “Glymphatic” Mechanism. J. Gen. Physiol. 2016, 148 (6), 489-501.
(87) Galgoczy, R.; Pastor, I.; Colom, A.; Giménez, A.; Mas, F.; Alcaraz, J. Colloids and Surfaces B: Biointerfaces A Spectrophotometer-Based Diffusivity Assay Reveals That Diffusion Hindrance of Small Molecules in Extracellular Matrix Gels Used in 3D Cultures Is Dominated by Viscous Effects. Colloids Surfaces B Biointerfaces 2014, 120, 200-207.
(88) Dancy, J. G.; Wadajkar, A. S.; Schneider, C. S.; Mauban, J. R. H.; Goloubeva, O. G.; Woodworth, G. F.; Winkles, J. A.; Kim, A. J. Non-Specific Binding and Steric Hindrance Thresholds for Penetration of Particulate Drug Carriers within Tumor Tissue. J. Control. Release 2016, 238, 139-148.
(89) Tomasetti, L.; Liebl, R.; Wastl, D. S.; Breunig, M. Influence of PEGylation on Nanoparticle Mobility in Different Models of the Extracellular Matrix. Eur. J. Pharm. Biopharm. 2016, 108, 145-155.
(90) Wong, C.; Stylianopoulos, T.; Cui, J.; Martin, J.; Chauhan, V. P.; Jiang, W.; Popovic, Z.; Jain, R. K.; Bawendi, M. G.; Fukumura, D. Multistage Nanoparticle Delivery System for Deep Penetration into Tumor Tissue. Proc. Natl. Acad. Sci. 2011, 108 (6), 2426-2431.
(91) Wong, C.; Stylianopoulos, T.; Cui, J.; Martin, J.; Chauhan, V. P.; Jiang, W.; Popovic, Z.; Jain, R. K.; Bawendi, W. G.; Fukumura, D. Multistage Nanoparticle Delivery System for Deep Penetration into Tumor Tissue. Proc. Nat. Acad. Sci. 2011, 108 (6), 2426-2431.
(92) Clauss, M. A.; Jain, R. K. Interstitial Transport of Rabbit and Sheep Antibodies in Normal and Neoplastic Tissues. Cancer Res. 1990, 50 (14), 3487-3492.
(93) Wang, C. K.; Craik, D. J. Cyclic Peptide Oral Bioavailability: Lessons from the Past. Biopolymers 2016, 106 (6), 901-909.
(94) Price, T. O.; Samson, W. K.; Niehoff, M. L.; Banks, W. A. Permeability of the Blood-Brain Barrier to a Novel Satiety Molecule Nesfatin-1. Peptides 2007, 28 (12), 2372-2381.
(95) Urayama, A.; Grubb, J. H.; Banks, W. A.; Sly, W. S. Epinephrine Enhances Lysosomal Enzyme Delivery across the Blood Brain Barrier by Up-Regulation of the Mannose 6-Phosphate Receptor. Proc. Nat. Acad. Sci. 2007, 104 (31), 12873-12878.
(96) Urayama, A.; Grubb, J. H.; Sly, W. S.; Banks, W. A. Mannose 6-Phosphate Receptor-Mediated Transport of Sulfamidase across the Blood-Brain Barrier in the Newborn Mouse. Mol. Ther. 2008, 16 (7), 1261-1266.
(97) Schatz, P. J. Use of Peptide Libraries to Map the Substrate Specificity of a Peptide-Modifying Enzyme: A 13 Residue Consensus Peptide Specifies Biotinylation in Escherichia Coli. Nat. Biotechnol. 1993, 11, 1138-1143.
(98) Ghosh, D.; Kohli, A. G.; Moser, F.; Endy, D.; Belcher, A. M. Refactored M13 Bacteriophage as a Platform for Tumor Cell Imaging and Drug Delivery. ACS Synth. Biol. 2012, 1 (12), 576-582.
(99) Scholle, M. D.; Kriplani, U.; Pabon, A.; Sishtla, K.; Glucksman, M. J.; Kay, B. K. Mapping Protease Substrates by Using a Biotinylated Phage Substrate Library. ChemBioChem 2006, No. 8, 834-838.
(100) Kim, J.; Adhikari, M.; Dhamane, S.; Hagstro, A. E. V; Kourentzi, K.; Strych, U.; Willson, R. C.; Conrad, J. C. Detection of Viruses By Counting Single Fluorescent Genetically Biotinylated Reporter Immunophage Using a Lateral Flow Assay. ACS Appl. Mater. Interfaces 2015, 7, 2891-2898.
(101) Banks, W. A.; Goulet, M.; Rusche, J. R.; Niehoff, M. L.; Boismenu, R. Differential Transport of a Secretin Analog across the Blood-Brain and Blood-Cerebrospinal Fluid Barriers of the Mouse. J. Pharmacol. Exp. Ther. 2002, 302 (3), 1062-1069.
(102) Smith, M. W.; Al-Jayyoussi, G.; Gumbleton, M. Peptide Sequences Mediating Tropism to Intact Blood-Brain Barrier: An in vivo Biodistribution Study Using Phage Display. Peptides 2012, 38 (1), 172-180.
(103) Mann, A. P.; Scodeller, P.; Hussain, S.; Braun, G. B.; Mölder, T.; Toome, K.; Ambasudhan, R.; Teesalu, T.; Lipton, S. A.; Ruoslahti, E. Cerebrovascular Changes in Mouse Models of Alzheimer's Disease. Nat. Commun. 2017, 8 (1), 1403.
(104) Deguchi, Y.; Kurihara, A.; Pardridge, W. M. Retention of Biologic Activity of Human Epidermal Growth Factor Following Conjugation to a Blood-Brain Barrier Drug Delivery Vector via an Extended Poly(Ethylene Glycol) Linker. Bioconjug. Chem. 1999, 10 (1), 32-37.
(105) Kurihara, A.; Pardridge, W. M. Imaging Brain Tumors by Targeting Peptide Radiopharmaceuticals through the Blood-Brain Barrier. Cancer Res. 1999, 59 (24), 6159-6163.
(106) Park, J.; Aryal, M.; Vykhodtseva, N.; Zhang, Y. Z.; McDannold, N. Evaluation of Permeability, Doxorubicin Delivery, and Drug Retention in a Rat Brain Tumor Model after Ultrasound-Induced Blood-Tumor Barrier Disruption. J. Control. Release 2017, 250, 77-85.
(107) Siegal, T.; Rubinstein, R.; Bokstein, F.; Schwartz, A.; Lossos, A.; Shalom, E.; Chisin, R.; Gomori, J. M. In vivo Assessment of the Window of Barrier Opening after Osmotic Blood-Brain Barrier Disruption in Humans. J. Neurosurg. 2000, 92 (4), 599-605.
(108) Zafir-Lavie, I.; Sherbo, S.; Goltsman, H.; Badinter, F.; Yeini, E.; Ofek, P.; Miari, R.; Tal, O.; Liran, A.; Shatil, T.; et al. Successful Intracranial Delivery of Trastuzumab by Gene-Therapy for Treatment of HER2-Positive Breast Cancer Brain Metastases. J. Control. Release 2018, 291, 80-89.
(109) Staquicini, F. I.; Ozawa, M. G.; Moya, C. A.; Driessen, W. H. P.; Barbu, E. M.; Nishimori, H.; Soghomonyan, S.; Flores, L. G.; Liang, X.; Paolillo, V.; et al. Systemic Combinatorial Peptide Selection Yields a Non-Canonical Iron-Mimicry Mechanism for Targeting Tumors in a Mouse Model of Human Glioblastoma. J. Clin. Invest. 2011, 121 (1), 161-173.
(110) Weksler, B. B.; Subileau, E. A.; Holloway, K.; Leveque, M.; Nicotra, A.; Bourdoulous, S.; Turowski, P.; Male, D. K.; Roux, F.; Greenwood, J.; et al. Blood-Brain Barrier-Specific Properties of a Human Adult Brain Endothelial Cell Line. 2005, 19 (13), 1872-1874.
(111) web.expasy.org/protparam/
(112) Dahan, M.; Levi, S.; Luccardini, C.; Rostaing, P.; Riveau, B.; Triller, A. Diffusion Dynamics of Glycine Receptors Revealed by Single-Quantum Dot Tracking. Science (80-.). 2003, 302 (5644), 442-445.
(113) Andrews, N. L.; Lidke, K. a; Pfeiffer, J. R.; Burns, A. R.; Bridget, S.; Oliver, J. M.; Lidke, D. S. Actin Restricts FcRI Diffusion and Facilitates Antigen-Induced Receptor Immobilisation. Nat cell biol 2008, 10 (8), 955-963.
(114) Li, N.; Hill, K. S.; Elferink, L. A. Analysis of Receptor Tyrosine Kinase Internalization Using Flow Cytometry. Methods Mol. Biol. 2008, 457, 305-317.
(115) Galgoczy, R.; Pastor, I.; Colom, A.; Giménez, A.; Mas, F.; Alcaraz, J. A Spectrophotometer-Based Diffusivity Assay Reveals That Diffusion Hindrance of Small Molecules in Extracellular Matrix Gels Used in 3D Cultures Is Dominated by Viscous Effects. Colloids Surfaces B Biointerfaces 2014, 120, 200-207.
(116) Raghavan, S.; Shen, C. J.; Desai, R. A.; Sniadecki, N. J.; Nelson, C. M.; Chen, C. S. Decoupling Diffusional from Dimensional Control of Signaling in 3D Culture Reveals a Role for Myosin in Tubulogenesis. J. Cell Sci. 2010, 123 (17), 2877-2883.
(117) Kleinman, H. K.; Martin, G. R. Matrigel: Basement Membrane Matrix with Biological Activity. Semin. Cancer Biol. 2005, 15, 378-386.
(118) Hughes, C. S.; Postovit, L. M.; Lajoie, G. A. Matrigel: A Complex Protein Mixture Required for Optimal Growth of Cell Culture. Proteomics 2010, 10 (9), 1886-1890.
(119) Urayama, A.; Grubb, J. H.; Sly, W. S.; Banks, W. A. Developmentally Regulated Mannose 6-Phosphate Receptor-Mediated Transport of a Lysosomal Enzyme across the Blood-Brain Barrier. 2004, 101 (34), 6-11.
(120) Triguero, D.; Buciak, J.; Pardridge, W. M. Capillary Depletion Method for Quantification of Blood-Brain Barrier Transport of Circulating Peptides and Plasma Proteins. J. Neurochem. 1990, 1882-1888.
(121) Johnson, B. M.; Zhang, P.; Schuetz, J. D.; Brouwer, K. L. R. Characterization of Transport Protein Expression in Multidrug Resistance-Associated Protein (MRP)2-Deficient Rats. Drug Metab. Dispos. 2006, 34 (5), 556-562.
(122) World-wide-web at neb.com/protocols/2014/05/08/m13-titer-protocol.

### Examples 4-6

Abbott, N. J., Patabendige, A. A. K., Dolman, D. E. M., Yusof, S. R., Begley, D. J., 2010. Structure and function of the blood-brain barrier. Neurobiol. Dis. 37, 13-25.
Buss, N. A. P. S., Henderson, S. J., McFarlane, M., Shenton, J. M., De Haan, L., 2012. Monoclonal antibody therapeutics: History and future. Curr. Opin. Pharmacol. 12, 615-622.
Chang, H. Y., Morrow, K., Bonacquisti, E., Zhang, W. Y., Shah, D. K., 2018. Antibody pharmacokinetics in rat brain determined using microdialysis. MAbs 10, 843-853.
Demeule, M., Regina, A., Ché, C., Poirier, J., Nguyen, T., Gabathuler, R., Castaigne, J.-P., Béliveau, R., 2008. Identification and design of peptides as a new drug delivery system for the brain. J. Pharmacol. Exp. Ther. 324, 1064-1072.
Elliott, J. M., Zhang, Y., Dennis, M. S., Yu, Y. J., Atwal, J., Hoyte, K., Lu, Y., Luk, W., Watts, R. J., Prabhu, S., Kenrick, M., 2011. Boosting Brain Uptake of a Therapeutic Antibody by Reducing Its Affinity for a Transcytosis Target. Sci. Transl. Med. 3, 84ra44-84ra44.
Fisher, A. C., Lee, S. L., Harris, D. P., Buhse, L., Kozlowski, S., Yu, L., Kopcha, M., Woodcock, J., 2016. Advancing pharmaceutical quality: An overview of science and research in the U. S. FDA's Office of Pharmaceutical Quality. Int. J. Pharm. 515, 390-402.
Freskgågrd, P. O., Urich, E., 2017. Antibody therapies in CNS diseases. Neuropharmacology 120, 38-55.
Ghosh, D., Peng, X., Leal, J., Mohanty, R. P., 2018. Peptides as drug delivery vehicles across biological barriers. J. Pharm. Investig. 48, 89-111.
Kaplon, H., Reichert, J. M., 2019. Antibodies to watch in 2019. MAbs 11, 219-238.
Kirley, T. L., Greis, K. D., Norman, A. B., 2018. Domain unfolding of monoclonal antibody fragments revealed by non-reducing SDS-PAGE. Biochem. Biophys. Reports 16, 138-144.
Najdekr, L., Friedecký, D., Tautenhahn, R., Pluskal, T., Wang, J., Huang, Y., Adam, T., 2016. Influence of mass resolving power in orbital ion-trap mass spectrometry-based metabolomics. Anal. Chem. 88, 11429-11435.
Nebija, D., Kopelent-Frank, H., Urban, E., Noe, C. R., Lachmann, B., 2011. Comparison of two-dimensional gel electrophoresis patterns and MALDI-TOF MS analysis of therapeutic recombinant monoclonal antibodies trastuzumab and rituximab. J. Pharm. Biomed. Anal. 56, 684-691.
NIH, U. S. National Library of Medicine, ClinicalTrials.gov [WWW Document], n.d. URL Oller-Salvia, B., Sanchez-Navarro, M., Giralt, E., Teixid6, M., 2016. Blood-brain barrier shuttle peptides: An emerging paradigm for brain delivery. Chem. Soc. Rev. 45, 4690-4707.
Pardridge, W. M., 2017. Delivery of Biologics Across the Blood-Brain Barrier with Molecular Trojan Horse Technology. BioDrugs 31, 503-519.
Pardridge, W. M., 2016. Re-engineering therapeutic antibodies for Alzheimer's disease as blood-brain barrier penetrating bi-specific antibodies. Expert Opin. Biol. Ther. 16, 1455-1468.
Pardridge, W. M., Boado, R. J., Giugliani, R., Schmidt, M., 2018a. Plasma Pharmacokinetics of Valanafusp Alpha, a Human Insulin Receptor Antibody-Iduronidase Fusion Protein, in Patients with Mucopolysaccharidosis Type I. BioDrugs 32, 169-176.
Pardridge, W. M., Boado, R. J., Patrick, D. J., Hui, E. K. W., Lu, J. Z., 2018b. Blood-Brain Barrier Transport, Plasma Pharmacokinetics, and Neuropathology Following Chronic Treatment of the Rhesus Monkey with a Brain Penetrating Humanized Monoclonal Antibody Against the Human Transferrin Receptor. Mol. Pharm. 15, 5207-5216.
Peng, X., Liu, X., Liu, Y., Kim, J. Y., Chen, Y., Ang, P., n.d. Brain-penetrating peptide shuttles across the blood-brain barrier and extracellular-like space. ChemRxiv posted Dec. 6, 2019.
Rane, S. S., Dearman, R. J., Kimber, I., Uddin, S., Bishop, S., Shah, M., Podmore, A., Pluen, A., Derrick, J. P., 2019. Impact of a Heat Shock Protein Impurity on the Immunogenicity of Biotherapeutic Monoclonal Antibodies. Pharm. Res. 36, 1-14.
Rathnayaka, H., Mozziconacci, O., Sreedhara, A., Schöneich, C., 2018. Fragmentation of a Monoclonal Antibody by Peroxotungstate. Pharm. Res. 35, 1-15.
Regina, A., Demeule, M., Tripathy, S., Lord-Dufour, S., Currie, J.-C., Iddir, M., Annabi, B., Castaigne, J.-P., Lachowicz, J. E., 2015a. ANG4043, a novel brain-penetrant peptide-mAb conjugate, is efficacious against HER2-positive intracranial tumors in mice. Mol. Cancer Ther. 14, 129-40.
Regina, A., Demeule, M., Tripathy, S., Lord-dufour, S., Currie, J., Iddir, M., Annabi, B., Castaigne, J., Lachowicz, J. E., 2015b. ANG4043, a Novel Brain-Penetrant Peptide-mAb Conjugate, Is Efficacious against HER2-Positive Intracranial Tumors in Mice. Mol. Cancer Ther. 14, 129-140.
Sehlin, D., Fang, X. T., Meier, S. R., Jansson, M., Syvänen, S., 2017. Pharmacokinetics, biodistribution and brain retention of a bispecific antibody-based PET radioligand for imaging of amyloid-D. Sci. Rep. 7, 1-10.
Shreiber, A. M., 2019. Erenumab (Aimovig) for Migraine Prophylaxis in Adults. Am. Fam. Physician 99, 781-782.
Smolders, J., Heutinck, K. M., Fransen, N. L., Remmerswaal, E. B. M., Hombrink, P., ten Berge, I. J. M., van Lier, R. A. W., Huitinga, I., Hamann, J., 2018. Tissue-resident memory T cells populate the human brain. Nat. Commun. 9, 1-14.
Stetefeld, J., McKenna, S. A., Patel, T. R., 2016. Dynamic light scattering: a practical guide and applications in biomedical sciences. Biophys. Rev. 8, 409-427.
Takayama, Y., Kusamori, K., Nishikawa, M., 2019. Click Chemistry as a Tool for Cell Engineering and Drug Delivery. Molecules 24, 1-20.
Triguero, D., Buciak, J., Pardridge, W. M., 1990. Capillary Depletion Method for Quantification of Blood-Brain Barrier Transport of Circulating Peptides and Plasma Proteins. J. Neurochem. 1882-1888.
Villasenõr, R., Ozmen, L., Messaddeq, N., Grüninger, F., Loetscher, H., Keller, A., Betsholtz, C., Freskgård, P. O., Collin, L., 2016. Trafficking of Endogenous Immunoglobulins by Endothelial Cells at the Blood-Brain Barrier. Sci. Rep. 6, 1-10.
Yaldizli, O., Putzki, N., 2009. Natalizumab in the treatment of multiple sclerosis. Ther. Adv. Neurol. Disord. 2, 115-128.
Yuan, H., Lauritsen, C. G., Kaiser, E. A., Silberstein, S. D., 2017. CGRP Monoclonal Antibodies for Migraine: Rationale and Progress. BioDrugs 31, 487-501.

What is claimed is:

1. A peptide of from 7 to 25 amino acid residues comprising and comprising a sequence selected from SEQ ID NOS: 1-20, wherein said peptide further comprises or is linked to one or more of:
(a) a non-natural amino acid;
(b) a D-amino acid;
(c) a non-amino acid chemical feature; and/or
(d) a therapeutic or diagnostic payload.

2. The peptide of claim 1, wherein said peptide comprises one or more non-natural amino acid.

3. The peptide of claim 1, wherein said peptide comprises a D-amino acid.

4. The peptide of claim 3, wherein said peptide has more than one D-amino amino acid.

5. The peptide of claim 4, wherein said peptide comprises only D-amino-acids.

6. The peptide of claim 1, wherein said non-amino acid chemical feature is polyethylene glycol.

7. The peptide of claim 1, wherein said non-amino acid chemical feature is a linking agent.

8. The peptide of claim 1, wherein said payload is a therapeutic payload.

9. The peptide of claim 1, wherein said payload is a diagnostic payload.

10. The peptide of claim 1, wherein said peptide comprises (a) and (b); (a) and (c); (a) and (d); (b) and (c); (b) and (d); (c) and (d); (a), (b) and (c); (a), (c) and (d); (a), (b) and (d); (b), (c) and (d); or (a), (b), (c) and (d).

11. The peptide of claim 1, wherein said peptide is 8-25 residues in length, 9-25 residues in length, 10-25 residues in length, 12-25 residues in length, 15-25 residues in length or 20-25 residues in length.

12. The peptide of claim 1, wherein said peptide is 8-20 residues in length, 9-20 residues in length, 10-20 residues in length, 12-20 residues in length, or 15-20 residues in length.

13. The peptide of claim 1, wherein said peptide is 8-15 residues in length, 9-15 residues in length, 10-15 residues in length, or 12-15 residues in length.

14. The peptide of claim 1, wherein said peptide is 8, 9, 10, 11, 12, 13, 14, 15, 16, 17, 18, 19, 20, 21, 22, 23, 24 or 25 residues in length.

15. The peptide of claim 1, wherein said peptide is 7 residues in length, comprises at least one D-amino acid, and optionally is 9 residues in length, cyclized, such as through N-and C-terminal cysteine residues.

16. A method of delivering a therapeutic or diagnostic payload across the blood-brain barrier of a subject comprising administering to said subject a peptide of from 7 to 25 amino acid residues comprising and comprising a sequence selected from SEQ ID NOS: 1-20, wherein said peptide is linked to a therapeutic or diagnostic payload.

17. The method of claim 16, wherein said peptide comprises one or more of:
(a) a non-natural amino acid;
(b) a D-amino acid; and/or
(c) a non-amino acid chemical feature.

18. The method of claim 17, wherein said peptide comprises one or more non-natural amino acid.

19. The method of claim 17, wherein said peptide comprises a D-amino acid.

20. The method of claim 19, wherein said peptide has more than one D-amino amino acid, such as comprising only D-amino-acids.

21. The method of claim 17, wherein said non-amino acid chemical feature is polyethylene glycol.

22. The method of claim 17, wherein said non-amino acid chemical feature is a linking agent.

23. The method of claim 16, wherein said payload is a therapeutic payload.

24. The method of claim 16, wherein said payload is a diagnostic payload.

25. The method of claim 17, wherein said peptide comprises (a) and (b); (a) and (c); (b) and (c); or (a), (b) and (c).

26. The method of claim 16, wherein said peptide is 8-25 residues in length, 9-25 residues in length, 10-25 residues in length, 12-25 residues in length, 15-25 residues in length or 20-25 residues in length.

27. The method of claim 16, wherein said peptide is 8-20 residues in length, 9-20 residues in length, 10-20 residues in length, 12-20 residues in length, or 15-20 residues in length.

28. The method of claim 16, wherein said peptide is 8-15 residues in length, 9-15 residues in length, 10-15 residues in length, or 12-15 residues in length.

29. The method of claim 16, wherein said peptide is 8, 9, 10, 11, 12, 13, 14, 15, 16, 17, 18, 19, 20, 21, 22, 23, 24 or 25 residues in length.

30. The method of claim 16, wherein said peptide is 7 residues in length, comprises at least one D-amino acid, carries a therapeutic or diagnostic payload, and optionally is 9 residues in length, cyclized, such as through N- and C-terminal cysteine residues.

31. A method of treating a disease or disorder in a subject comprising administering to said subject a peptide of from 8 to 25 amino acid residues comprising and comprising a sequence selected from SEQ ID NOS: 1-20, wherein said peptide is linked to a therapeutic payload.

32. The method of claim 31, wherein said disease or disorder is a neurologic disease such as Alzheimer's Disease or Parkinson's Disease.

33. The method of claim 31, wherein said disease or disorder is stroke or traumatic brain injury.

34. The method of claim 31, wherein said disease or disorder is cancer, such as a glioma, a craniopharyngioma, a lymphoma, a haemangioblastoma, a meningioma, an acoustic neuroma, a pineal region tumor, a pituitary tumor, or a primitive neuroectodermal tumor.

35. The method of claim 31, wherein said peptide is administered orally, intravenously, intra-arterially, subcutaneously, or intramuscularly.

36. The method of claim 31, wherein said peptide is administered to said subject more than once.

37. The method of claim 36, wherein said peptide is administered daily, every other day, every three days, twice-weekly, weekly, every other week, or monthly.

38. The method of claim 36, wherein said peptide is administered on a chronic basis.

39. The method of claim 31, wherein said peptide further comprises one or more of:
(a) a non-natural amino acid;
(b) a D-amino acid; and/or
(c) a non-amino acid chemical feature.

40. The method of claim 31, wherein said peptide is 7 residues in length, comprises at least one D-amino acid, carries a therapeutic or diagnostic payload, and optionally is 9 residues in length, cyclized, such as through N- and C-terminal cysteine residues.

41. A method of diagnosing a disease or disorder in a subject comprising administering to said subject a peptide of from 8 to 25 amino acid residues comprising and comprising a sequence selected from SEQ ID NOS: 1-20, wherein said peptide is linked to a diagnostic payload.

42. The method of claim 41, wherein said disease or disorder is a neurologic disease such as Alzheimer's Disease or Parkinson's Disease, stroke or traumatic brain injury, or cancer.

43. The method of claim 41, wherein said peptide is administered orally, intravenously, intra-arterially, subcutaneously, or intramuscularly.

44. The method of claim 41, wherein said peptide further comprises one or more of:
(a) a non-natural amino acid;
(b) a D-amino acid; and/or
(c) a non-amino acid chemical feature.

45. The method of claim 41, wherein said peptide is 7 residues in length, comprises at least one D-amino acid, carries a therapeutic or diagnostic payload, and optionally is 9 residues in length, cyclized, such as through N- and C-terminal cysteine residues.


SEQUENCE LISTING (28 sequences)

SEQ ID NO: 1 - protein, 7 aa, Artificial sequence
  Synthetic peptide
NAGHLSQ

SEQ ID NO: 2 - protein, 7 aa, Artificial sequence
  Synthetic peptide
SAYDRPL

SEQ ID NO: 3 - protein, 7 aa, Artificial sequence
  Synthetic peptide
NSHTQGK

SEQ ID NO: 4 - protein, 7 aa, Artificial sequence
  Synthetic peptide
TYLNSAK

SEQ ID NO: 5 - protein, 7 aa, Artificial sequence
  Synthetic peptide
VNQGSIG

SEQ ID NO: 6 - protein, 7 aa, Artificial sequence
  Synthetic peptide
NIKSSHV

SEQ ID NO: 7 - protein, 7 aa, Artificial sequence
  Synthetic peptide
VPSKPGL

SEQ ID NO: 8 - protein, 7 aa, Artificial sequence
  Synthetic peptide
NWMINKE

SEQ ID NO: 9 - protein, 7 aa, Artificial sequence
  Synthetic peptide
LWRPAAD

SEQ ID NO: 10 - protein, 9 aa, Artificial sequence
  Synthetic peptide
CSKEATPFC

SEQ ID NO: 11 - protein, 7 aa, Artificial sequence
  Synthetic peptide
SSKHEAT

SEQ ID NO: 12 - protein, 7 aa, Artificial sequence
  Synthetic peptide
IHSPTAL

SEQ ID NO: 13 - protein, 7 aa, Artificial sequence
  Synthetic peptide
LTAKHMQ

SEQ ID NO: 14 - protein, 7 aa, Artificial sequence
  Synthetic peptide
GPTAKYI

SEQ ID NO: 15 - protein, 7 aa, Artificial sequence
  Synthetic peptide
DGLAKNS

SEQ ID NO: 16 - protein, 7 aa, Artificial sequence
  Synthetic peptide
ISSSINH

SEQ ID NO: 17 - protein, 7 aa, Artificial sequence
  Synthetic peptide
NMHTPMV

SEQ ID NO: 18 - protein, 7 aa, Artificial sequence
  Synthetic peptide
TTKLPNS

SEQ ID NO: 19 - protein, 7 aa, Artificial sequence
  Synthetic peptide
MNQASMS

SEQ ID NO: 20 - protein, 7 aa, Artificial sequence
  Synthetic peptide
PKGDENT

SEQ ID NO: 21 - DNA, 55 bp, Artificial sequence
  Synthetic primer
tcgtcggcagcgtcagatgtgtataagagacagagcaagctgataaaccgataca

SEQ ID NO: 22 - DNA, 56 bp, Artificial sequence
  Synthetic primer
gtctcgtgggctcggagatgtgtataagagacagttgtcgtctttccagacgttag

SEQ ID NO: 23 - protein, 12 aa, Artificial sequence
  Synthetic peptide
THRPPMWSPVWP

SEQ ID NO: 24 - protein, 9 aa, Artificial sequence
  Synthetic peptide
CRTIGPSVC

SEQ ID NO: 25 - protein, 15 aa, Artificial sequence
  Synthetic peptide
GLNDIFEAQKIEWHE

SEQ ID NO: 26 - protein, 19 aa, Artificial sequence
  Synthetic peptide
TFFYGGSRGKRNNFKTEEY

SEQ ID NO: 27 - protein, 9 aa, Artificial sequence
  Synthetic peptide
CLWRPAADC

SEQ ID NO: 28 - DNA, 27 bp, Artificial sequence
  Synthetic oligonucleotide
gaacctccaccgcagttcccctgagta